(19) United States
(12) Patent Application Publication
(10) Pub. No.: US 2003/0190715 A1
(43) Pub. Date: Oct. 9, 2003

(54) Novel proteins and nucleic acids encoding same

(76) Inventors: William M. Grosse, Branford, CT (US); John P. Alsobrook II, Madison, CT (US); Denise M. Lepley, Branford, CT (US); Catherine E. Burgess, Wethersfield, CT (US); Vishnu Mishra, Gainesville, FL (US); Ramesh Kekuda, Stamford, CT (US); Li Li, Branford, CT (US); Muralidhara Padigaru, Branford, CT (US); Richard A. Shimkets, West Haven, CT (US); Bryan D. Zerhusen, Branford, CT (US); Kimberly A. Spytek, New Haven, CT (US); Shlomit R. Edinger, New Haven, CT (US); Valerie Gerlach, Branford, CT (US); John R. MacDougall, Hamden, CT (US); Isabelle Millet, Milford, CT (US); David J. Stone, Guilford, CT (US); Erik Gunther, Branford, CT (US); Karen Ellerman, Branford, CT (US)

Correspondence Address: Ivor R. Elrifi, Mintz, Levin, Cohn, Ferris,, Glovsky and Popeo, P.C., One Financial Center, Boston, MA, 02111, US

(21) Appl. No.: 09/976,782

(22) Filed: Oct. 12, 2001

Related U.S. Application Data
(60) Provisional application No. 60/240,113, filed on Oct. 12, 2000.
(60) Provisional application No. 60/240,662, filed on Oct. 16, 2000.
(60) Provisional application No. 60/240,732, filed on Oct. 16, 2000.
(60) Provisional application No. 60/240,625, filed on Oct. 16, 2000.
(60) Provisional application No. 60/240,648, filed on Oct. 16, 2000.
(60) Provisional application No. 60/240,703, filed on Oct. 16, 2000.
(60) Provisional application No. 60/241,190, filed on Oct. 16, 2000.
(60) Provisional application No. 60/240,637, filed on Oct. 16, 2000.
(60) Provisional application No. 60/240,669, filed on Oct. 16, 2000.
(60) Provisional application No. 60/262,455, filed on Jan. 18, 2001.

(51) Int. Cl. C12N 9/00; C07H 21/04; C12P 21/02; C12N 5/06

(52) U.S. Cl. 435/183; 435/69.1; 435/325; 435/320.1; 530/350; 536/23.2

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants, or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/240,113, filed Oct. 12, 2000; U.S. Ser. No. 60/240,662, filed Oct. 16, 2000; U.S. Ser. No. 60/240,732, filed Oct. 16, 2000; U.S. Ser. No. 60/240,625 filed Oct. 16, 2000; U.S. Ser. No. 60/240,648, filed Oct. 16, 2000; U.S. Ser. No. 60/240,703, filed Oct. 16, 2000; U.S. Ser. No. 60/241,190, filed Oct. 16, 2000; U.S. Ser. No. 60/240,637, filed Oct. 16, 2000; U.S. Ser. No. 60/240,669, filed Oct. 16, 2000; and U.S. Ser. No. 60/262,455, filed Jan. 18, 2001, each of which is incorporated by reference in its entirety.

### FIELD OF THE INVENTION

The invention generally relates to nucleic acids and polypeptides encoded thereby.

### BACKGROUND OF THE INVENTION

The invention generally relates to nucleic acids and polypeptides encoded therefrom. More specifically, the invention relates to nucleic acids encoding cytoplasmic, nuclear, membrane bound, and secreted polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as NOVX, or NOV1, NOV2, NOV3, NOV4, NOV5, NOV6, NOV7, NOV8, and NOV9 nucleic acids and polypeptides. These nucleic acids and polypeptides, as well as derivatives, homologs, analogs and fragments thereof, will hereinafter be collectively designated as “NOVX” nucleic acid or polypeptide sequences.

In one aspect, the invention provides an isolated NOVX nucleic acid molecule encoding a NOVX polypeptide that includes a nucleic acid sequence that has identity to the nucleic acids disclosed in SEQ ID NOS: 1,3,5,7,9,11,13,15,17,19,21,23, and 25. In some embodiments, the NOVX nucleic acid molecule will hybridize under stringent conditions to a nucleic acid sequence complementary to a nucleic acid molecule that includes a protein-coding sequence of a NOVX nucleic acid sequence. The invention also includes an isolated nucleic acid that encodes a NOVX polypeptide, or a fragment, homolog, analog or derivative thereof. For example, the nucleic acid can encode a polypeptide at least 80% identical to a polypeptide comprising the amino acid sequences of SEQ ID NOS: 2, 4, 6, 8, 10, 12, 14, 16, 18, 20, 22, 24 and 26. The nucleic acid can be, for example, a genomic DNA fragment or a cDNA molecule that includes the nucleic acid sequence of any of SEQ ID NOS: 1, 3, 5, 7, 9, 11, 13, 15, 17, 19, 21, 23, and 25.

Also included in the invention is an oligonucleotide, e.g., an oligonucleotide which includes at least 6 contiguous nucleotides of a NOVX nucleic acid (e.g., SEQ ID NOS: 1, 3, 5, 7, 9, 11, 13, 15, 17, 19, 21, 23, and 25) or a complement of said oligonucleotide.

Also included in the invention are substantially purified NOVX polypeptides (SEQ ID NOS: 2, 4, 6, 8, 10, 12, 14, 16, 18, 20, 22, 24 and 26). In certain embodiments, the NOVX polypeptides include an amino acid sequence that is substantially identical to the amino acid sequence of a human NOVX polypeptide.

The invention also features antibodies that immunoselectively bind to NOVX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e.g., a NOVX nucleic acid, a NOVX polypeptide, or an antibody specific for a NOVX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a NOVX nucleic acid, under conditions allowing for expression of the NOVX polypeptide encoded by the DNA. If desired, the NOVX polypeptide can then be recovered.

In another aspect, the invention includes a method of detecting the presence of a NOVX polypeptide in a sample. In the method, a sample is contacted with a compound that selectively binds to the polypeptide under conditions allowing for formation of a complex between the polypeptide and the compound. The complex is detected, if present, thereby identifying the NOVX polypeptide within the sample.

The invention also includes methods to identify specific cell or tissue types based on their expression of a NOVX.

Also included in the invention is a method of detecting the presence of a NOVX nucleic acid molecule in a sample by contacting the sample with a NOVX nucleic acid probe or primer, and detecting whether the nucleic acid probe or primer bound to a NOVX nucleic acid molecule in the sample.

In a further aspect, the invention provides a method for modulating the activity of a NOVX polypeptide by contacting a cell sample that includes the NOVX polypeptide with a compound that binds to the NOVX polypeptide in an amount sufficient to modulate the activity of said polypeptide. The compound can be, e.g., a small molecule, such as a nucleic acid, peptide, polypeptide, peptidomimetic, carbohydrate, lipid or other organic (carbon containing) or inorganic molecule, as further described herein.

Also within the scope of the invention is the use of a therapeutic in the manufacture of a medicament for treating or preventing disorders or syndromes including, e.g., Cancer, Leukodystrophies, Breast cancer, Ovarian cancer, Prostate cancer, Uterine cancer, Hodgkin disease, Adenocarcinoma, Adrenoleukodystrophy,Cystitis, incontinence, Von Hippel-Lindau (VHL) syndrome, hypercalceimia, Endometriosis, Hirschsprung's disease, Crohn's Disease, Appendicitis, Cirrhosis, Liver failure, Wolfram Syndrome, Smith-Lemli-Opitz syndrome, Retinitis pigmentosa, Leigh syndrome; Congenital Adrenal Hyperplasia, Xerostomia; tooth decay and other dental problems; Inflammatory bowel disease, Diverticular disease, fertility, Infertility, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, Hemophilia, Hypercoagulation, Idiopathic thrombocytopenic purpura, obesity, Diabetes Insipidus and Mellitus with Optic Atrophy and Deafness, Pancreatitis, Metabolic Dysregulation, transplantation recovery, Autoimmune disease, Systemic lupus erythematosus, asthma, arthritis, psoriasis, Emphysema, Scleroderma, allergy, ARDS, Immunodeficiencies, Graft vesus host, Alzheimer's disease, Stroke, Parkinson's disease, Huntington's disease, Cerebral palsy, Epilepsy, Multiple sclerosis,Ataxia-telangiectasia, Behavioral disorders, Addiction, Anxiety, Pain, Neurodegeneration, Muscular dystrophy,Lesch-Nyhan syndrome,Myasthenia gravis, schizophrenia, and other dopamine-dysfunctional states, levodopa-induced dyskinesias, alcoholism, pileptic seizures and other neurological disorders, mental depression, Cerebellar ataxia, pure; Episodic ataxia, type 2; Hemiplegic migraine, Spinocerebellar ataxia-6, Tuberous sclerosis, Renal artery stenosis, Interstitial nephritis, Glomerulonephritis, Polycystic kidney disease, Renal tubular acidosis, IgA nephropathy, and/or other pathologies and disorders of the like.

The therapeutic can be, e.g., a NOVX nucleic acid, a NOVX polypeptide, or a NOVX-specific antibody, or biologically-active derivatives or fragments thereof.

For example, the compositions of the present invention will have efficacy for treatment of patients suffering from the diseases and disorders disclosed above and/or other pathologies and disorders of the like. The polypeptides can be used as immunogens to produce antibodies specific for the invention, and as vaccines. They can also be used to screen for potential agonist and antagonist compounds. For example, a cDNA encoding NOVX may be useful in gene therapy, and NOVX may be useful when administered to a subject in need thereof. By way of non-limiting example, the compositions of the present invention will have efficacy for treatment of patients suffering from the diseases and disorders disclosed above and/or other pathologies and disorders of the like.

The invention further includes a method for screening for a modulator of disorders or syndromes including, e.g., the diseases and disorders disclosed above and/or other pathologies and disorders of the like. The method includes contacting a test compound with a NOVX polypeptide and determining if the test compound binds to said NOVX polypeptide. Binding of the test compound to the NOVX polypeptide indicates the test compound is a modulator of activity, or of latency or predisposition to the aforementioned disorders or syndromes.

Also within the scope of the invention is a method for screening for a modulator of activity, or of latency or predisposition to disorders or syndromes including, e.g., the diseases and disorders disclosed above and/or other pathologies and disorders of the like by administering a test compound to a test animal at increased risk for the aforementioned disorders or syndromes. The test animal expresses a recombinant polypeptide encoded by a NOVX nucleic acid. Expression or activity of NOVX polypeptide is then measured in the test animal, as is expression or activity of the protein in a control animal which recombinantly-expresses NOVX polypeptide and is not at increased risk for the disorder or syndrome. Next, the expression of NOVX polypeptide in both the test animal and the control animal is compared. A change in the activity of NOVX polypeptide in the test animal relative to the control animal indicates the test compound is a modulator of latency of the disorder or syndrome.

In yet another aspect, the invention includes a method for determining the presence of or predisposition to a disease associated with altered levels of a NOVX polypeptide, a NOVX nucleic acid, or both, in a subject (e.g., a human subject). The method includes measuring the amount of the NOVX polypeptide in a test sample from the subject and comparing the amount of the polypeptide in the test sample to the amount of the NOVX polypeptide present in a control sample. An alteration in the level of the NOVX polypeptide in the test sample as compared to the control sample indicates the presence of or predisposition to a disease in the subject. Preferably, the predisposition includes, e.g., the diseases and disorders disclosed above and/or other pathologies and disorders of the like. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a NOVX polypeptide, a NOVX nucleic acid, or a NOVX-specific antibody to a subject (e.g., a human subject), in an amount sufficient to alleviate or prevent the pathological condition. In preferred embodiments, the disorder, includes, e.g., the diseases and disorders disclosed above and/or other pathologies and disorders of the like.

In yet another aspect, the invention can be used in a method to identity the cellular receptors and downstream effectors of the invention by any one of a number of techniques commonly employed in the art. These include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences and their encoded polypeptides. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| NOVX | | NO | NO | |
| Assign- | Internal | (nucleic | (poly- | |
| ment | Identification | acid) | peptide) | Homology |
| 1 | sggc_final_dj697k14_2000 | 1 | 2 | TYROSINE-PROTEIN |
| | 0719/CG108678-03 | | | KINASE 6-like |
| 2a | AC058790_da1 | 3 | 4 | Keratin 4-like |
| 2b | AC058790_da2 | 5 | 6 | Keratin 4-like |
| 2c | AC058790_da3 | 7 | 8 | Keratin 4-like |
| 2d | AC058790_da4 | 9 | 10 | Keratin 4-like |
| 3 | SC10341332_A | 11 | 12 | Collagen-like |
| 4 | GMAC018494_A | 13 | 14 | Cystatin B-like |
| 5 | GMAC009404_A | 15 | 16 | Serotonin Receptor-like |
| 6a | SC126404196_A | 17 | 18 | Cold Inducible Glycoprotein 30-like |
| 6b | SC126404196_A_{—dal/} | 19 | 20 | Cold Inducible Glycoprotein 30-like |
| | CG55866-01 | | | |
| 7 | SC122984679_A | 21 | 22 | Matrilin-2 -like |
| 8 | SC65666665_A | 23 | 24 | Leucocyte Surface |
| | | | | Antigen (CD53)-like |
| 9 | GM358d14_A | 25 | 26 | Tyrosine kinase-like |

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

NOV1 is homologous to a tyrosine protein kinase-6-like family of proteins. Thus, the NOV1 nucleic acids, polypeptides, antibodies and related compounds according to the invention will be useful in therapeutic and diagnostic applications implicated in, for example; cancer, especially but not limited to, breast cancer, colorectal cancer and melanoma, and/or other pathologies/disorders.

NOV2 is homologous to the keratin-4-like family of proteins. Thus NOV2 nucleic acids, polypeptides, antibodies and related compounds according to the invention will be useful in therapeutic and diagnostic applications implicated in, for example; Steatocystoma multiplex, Muscular dystrophy, Lesch-Nyhan syndrome, Myasthenia gravis and Breast Cancer other muscular disorders and/or other pathologies/disorders.

NOV3 is homologous to a family of collagen-like proteins. Thus, the NOV3 nucleic acids and polypeptides, antibodies and related compounds according to the invention will be useful in therapeutic and diagnostic applications implicated in, for examplevascular disorders, hypertension, skin disorders, renal disorders including Alport syndrome, immunological disorders, inflammation including irritable bowel disease, and tissue injury, cancers, fibrosis disorders, bone diseases, Ehlers-Danlos syndrome type VI, VII, type IV, S-linked cutis laxa and Ehlers-Danlos syndrome type V, osteogenesis imperfecta, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, and Neuroprotection and/or other pathologies/disorders.

NOV4 is homologous to the cystatin B-like family of proteins. Thus, NOV4 nucleic acids, polypeptides, antibodies and related compounds according to the invention will be useful in therapeutic and diagnostic applications implicated in, for example Alzheimer's disease (AD). Epilepsies, Unverricht-Lundborg disease; skin disorders, differentiation of keratinocytes; Cerebral amyloid angiopathy (CAA), amyloidosis, and hemorrhagic stroke; inflammatory disorders, allergic inflammation; cancer; HIV and AIDS; kidney diseases; Neurological disorders and/or other pathologies/disorders.

NOV5 is homologous to the serotonin receptor-like family of proteins. Thus NOV5 nucleic acids, polypeptides, antibodies and related compounds according to the invention will be useful in therapeutic and diagnostic applications implicated in, for example: from migraine, Alzheimer disease, eating disorder, anxiety-related disorder, epilepsy, retinoblastoma, schizophrenia, Tourette syndrome, autistic disorder, heart disorders and/or other pathologies/disorders.

NOV6 is homologous to the cold inducible glycoprotein 30-like family of proteins. Thus NOV6 nucleic acids, polypeptides, antibodies and related compounds according to the invention will be useful in therapeutic and diagnostic applications implicated in, for example: Lipoprotein disorder, cirrhosis, and olivopontocerebellar degeneration, hypertrophic obstructive cardiomyopathy, recurrent nonimmune hydrops fetalis and/or other pathologies/disorders.

NOV7 is homologous to the matrilin-2-like family of proteins. Thus NOV7 nucleic acids, polypeptides, antibodies and related compounds according to the invention will be useful in therapeutic and diagnostic applications implicated in, for example: fibrosarcoma, multiple sclerosis, chondrodysplasias: hypochondroplasia, achondroplasia, autosomal dominant SED tarda, and multiple epiphyseal dysplasia, polychondritis, and/or other pathologies/disorders.

NOV8 is homologous to the leucocyte surface antigen (CD53)-like family of proteins. Thus, NOV8 nucleic acids and polypeptides, antibodies and related compounds according to the invention will be useful in therapeutic and diagnostic applications implicated in, for example; from cancer, autoimmune disease, and infectious diseases. These diseases include but are not limited to Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection, Endocrine dysfunctions, Diabetes, obesity, Growth and reproductive disorders, Myasthenia gravis, Hemophilia, hypercoagulation, Idiopathic thrombocytopenic purpura, autoimmume disease, allergies, immunodeficiencies, transplantation, Graft vesus host disease (GVHD), Anemia, Ataxia-telangiectasia, Autoimmume disease, Hypercoagulation, Idiopathic thrombocytopenic purpura, Lymphedema, Lymphaedema, and cancers including but not limited to bone cancer, brain cancer, and liver cancer and/or other pathologies/disorders.

NOV9 is homologous to the tyrosine kinase-like family of proteins. Thus NOV7 nucleic acids, polypeptides, antibodies and related compounds according to the invention will be useful in therapeutic and diagnostic applications implicated in, for example: from breast cancer, other types of cancer, immunological disorders, haematological disorders, myelodysplastic syndrome and/or other pathologies/disorders.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit, e.g., neurogenesis, cell differentiation, cell proliferation, hematopoiesis, wound healing and angiogenesis.

Additional utilities for the NOVX nucleic acids and polypeptides according to the invention are disclosed herein.

NOV1

NOV1 includes a novel tyrosine protein kinase 6-like protein disclosed below. A disclosed NOV1 nucleic acid of 1345 nucleotides (also referred to as sggc_final_dj697k14_{—}20000719 or CG108678-03) encoding a novel tyrosine protein kinase 6-like protein is shown in Table 1A. An open reading frame was identified beginning with an ATG initiation codon at nucleotides 29-31 and ending with a TGA codon at nucleotides 1340-1342. A putative untranslated region upstream from the initiation codon and downstream from the termination codon is underlined in Table 1A. The start and stop codons are in bold letters.

TABLE 1A
NOV1 nucleotide sequence.

(SEQ ID NO:1)

CCTGGTCCTGCCGCTGCGCCCCGCCCGCCATGGTGTCCCGGGACCAGGCTCACCTGGGCCCCAAGTATGTGGGCCTCTGG
ACTTCAAGTCCCGGACGGACGAGGAGCTGAGCTTCCGCGCGGGGGACGTCTTCCACGTGGCCAGGAAGGAGGAGCAGTGG
TGGTGGGCCACGCTGCTGGACGAGGCGGGTGGGGCCGTGGCCCAGGGCTATGTGCCCCACAACTACCTGGCCGAGAGGGA
GACGGTGGAGTCGGAACCGTGGTTCTTTGGCTGCATCTCCCGCTCGGAAGCTGTGCGTCGGCTGCAGGCCGAGGGCAACG
CCACGGGCGCCTTCCTGATCAGGGTCAGCGAGAAGCCGAGTGCCGACTACGTCCTGTCGGTGCGGGACAGCAGGCTGTG
CGGCACTACAAGATCTGGCGGCGTGCCGGGGGCCGGCTGCACCTGAACGAGGCGGTGTCCTTCCTCAGCCTGCCCGAGCT
TGTGAACTACCACAGGGCCCAGAGCCTGTCCCACGGCCTGCGGCTGGCCGCGCCCTGCCGGAAGCACGAGCCTGAGCCCC
TGCCCCATTGGGATGACTGGGAGAGGCCGAGGGAGGAGTTCACGCTCTGCAGGAAGCTGGGGTCCGGCTACTTTGGGGAG
GTCTTCGAGGGGCTCTGGAAGACCGGGTCCAGGTGGCCATTAAGGTGATTTCTCGAGACAACCTCCTGCACCAGCAGAT
GCTGCAGTCGGAGATCCAGGCCATGAAGAAGCTGCGGCACAAACACATCCTGGCGCTGTACGCCGTGGTGTCCGTGGGG
ACCCCGTGTACATCATCACGGAGCTCATGGCCAAGGGCAGCCTGCTGGAGCTGCTCCGCGACTCTGATGAGAAAGTCCTG
CCCGTTTCGGAGCTGCTGGACATCGCCTGGCAGGTGGCTGAGGGCATGTGTTACCTGGAGTCGCAGAATTACATCCACCG
GGACCTGGCCGCCAGGAACATCCTCGTCGGGGAAAACACCCTCTGCAAAGTTGGGGACTTCGGGTTAGCCAGGCTTATCA
AGTGGACGGCCCCTGAGCGCTCTCCCGAGGCCATTACTCCACCAAATCCGACGTCTGGTCCTTTGGGATTCTCCTGCAT
GAGATGTTCAGCAGGGGTCAGGTGCCCTACCCAGGCATGTCCAACCATGAGGCCTTCCTGAGGGTGGACGCCGGCTACCG
CATGCCCTGCCCTCTGGAGTGCCCGCCCAGCGTGCACAAGCTGATGCTGACATGCTGGTGCAGGGACCCCGAGCAGAGAC
CCTGCTTCAAGGCCCTGCGGGAGAGGCTCTCCAGCTTCACCAGCTACGAGAACCCGACCTGAGCT

In a search of sequence databases, it was found, for example, that a NOV1 nucleic acid sequence, which maps to human chromosome 20, has 1213 of 1345 bases (90%) identical to a TYROSINE-PROTEIN KINASE 6 (EC 2.7.1.112) (BREAST TUMOR KINASE) (TYROSINE-PROTEIN KINASE BRK) mRNA from Homo sapiens (patn:Q81189). Public nucleotide databases include all GenBank databases and the GeneSeq patent database.

In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the NOV1 BLAST analysis, e.g., human tyrosine protein kinase BRK mRNA, matched the Query NOV1 sequence purely by chance is 1.8e−²³². The Expect value (E) is a parameter that describes the number of hits one can “expect” to see just by chance when searching a database of a particular size. It decreases exponentially with the Score (S) that is assigned to a match between two sequences. Essentially, the E value describes the random background noise that exists for matches between sequences.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. (Wootton and Federhen, Methods Enzymol 266:554-571, 1996).

The disclosed NOV1 polypeptide (SEQ ID NO: 2) encoded by SEQ ID NO: 1 has 437 amino acid residues and is presented in Table 1B using the one-letter amino acid code. Signal P, Psort and/or Hydropathy results predict that NOV1 does not have a signal peptide and is likely to be localized in the endoplasmic reticulum (membrane) with a certainty of 0.8500. In other embodiments, NOV1 may also be localized to the microbody with acertainty of 0.6177, the plasma membrane with a certainty of 0.4400, or to the mitochondrial inner membrane with a certainty of 0.1000.

Exon linking Data for NOV1 can be found below in Example 1. SNP data for NOV1 can be found below in Example 3.

TABLE 1B
Encoded NOV1 protein sequence.

(SEQ ID NO:2)

MVSRDQAHLGPKYVGLWDFKSRTDEELSFRAGDVFHVARKEEQWWWATLLDEAGGAVAQGYVPHNYLAERET
VESEPWFFGCISRSEAVRRLQAEGNATGAFLIRVSEKPSADYVLSVRDTQAVRHYKIWRRAGGRLHLNEAVS
FLSLPELVNYHRAQSLSHGLRLAAPCRKHEPEPLPHWDDWERPREEFTLCRKLGSGYFGEVFEGLWKDRVQV
AIKVISRDNLLHQQMLQSEIQAMKKLRHKHILALYAVVSVGDPVYIITELMAKGSLLELLRDSDEKVLPVSE
LLDIAWQVAEGMCYLESQNYIHRDLAARNILVGENTLCKVGDFGLARLIKWTAPEALSRGHYSTKSDVWSFG
ILLHEMFSRGQVPYPGMSNHEAFLRVDAGYRMPCPLECPPSVHKLMLTCWCRDPEQRPCFKALRERLSSFTS
YENPT

The full amino acid sequence of the disclosed NOV1 protein was found to have 437 of 451 amino acid residues (96%) identical to, and 437 of 451 amino acid residues (96%) similar to the 451 amino acid residue TYROSINE-PROTEIN KINASE 6 (EC 2.7.1.112) (BREAST TUMOR KINASE) (TYROSINE-PROTEIN KINASE BRK) from Homo sapiens (ptnr:SWISSNEW-ACC:Q13882). Public amino acid databases include the GenBank databases, SwissProt, PDB and PIR.

NOV1 is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus and breast (normal or tumor)

The disclosed NOV1 polypeptide has homology to the amino acid sequences shown in the BLAST data listed in Table 1C.

TABLE 1C
BLAST results for NOV1

| Gene | | | | | |
|---|---|---|---|---|---|
| Index/ | | Length | Identity | Positives | |
| Identifier | Protein/Organism | (aa) | (%) | (%) | Expect |
| 5174647 | PTK6 protein | 451 | 96 | 96 | 0.0 |
| | tyrosine | | | | |
| | kinase/human | | | | |
| 6677971 | Src-related | 451 | 77 | 86 | 0.0 |
| | kinase/mouse | | | | |
| 14769936 | PTK6 protein | 221 | 93 | 93 | e-117 |
| | tyrosine | | | | |
| | kinase/human | | | | |
| 174436 | SRK1 | 505 | 44 | 61 | e-105 |
| | kinase/Spongill | | | | |
| | lacustris | | | | |
| 125372 | FYN | 537 | 43 | 61 | e-105 |
| | kinase/Xiphophorus | | | | |
| | helleri | | | | |

The homology between these and other sequences is shown graphically in the ClustalW analysis shown in Table 1 D. In the ClustalW alignment of the NOV1 protein, as well as all other ClustalW analyses herein, the black outlined amino acid residues indicate regions of conserved sequence (i.e., regions that may be required to preserve structural or functional properties), whereas non-highlighted amino acid residues are less conserved and can potentially be altered to a much broader extent without altering protein structure or function.

[image]

[image]

The presence of identifiable domains in NOV1, as well as all other NOVX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results for NOV 1 as disclosed in Tables 1E-g, were collected from the Conserved Domain Database (CDD) with Reverse Position Specific BLAST analyses. This BLAST analysis software samples domains found in the Smart and Pfam collections. For Table 1E and all successive DOMAIN sequence alignments, filly conserved single residues are indicated by black shading or by the sign (|) and “strong” semi-conserved residues are indicated by grey shading or by the sign (+). The “strong” group of conserved amino acid residues may be any one of the following groups of amino acids: STA, NEQK, NHQK, NDEQ, QHRK, MILV, MILF, HY, FYW.

Tables 1E-G lists the domain description from DOMAIN analysis results against NOV1. This indicates that the NOV1 sequence has properties similar to those of other proteins known to contain this domain.

TABLE 1E
Domain Analysis of NOV1

gnl|Smart|smart00219, TyrKc, Tyrosine kinase, catalytic domain;
Phosphotransferases. Tyrosine-specific kinase subfamily.

| (SEQ ID NO: 2) | | | |
|---|---|---|---|
| NOV1: | 192 | TLCRKLGSGYFGEVFEGLWKDR----VQVAIKVISRDNLLHQ-QMLQSEIQAMKKLRHKH | 246 |
| (SEQ ID NO: 29) | | | |
| TyrKc: | 2 | TLGKKLGEGAFGEVYKGTLKGKGGVEVEVAVKTLKEDASEQQIEEFLREARLMRKLDHPN | 61 |
| NOV1: | 247 | ILALYAVVSVGDPVYIITELMAKGSLLELLRDSDEKVLPVSELLDIAWQVAEGMCYLESQ | 306 |
| TyrKc: | 62 | IVKLLGVCTEEEPLMIVMEYMEGGDLLDYLRKNRPKELSLSDLLSFALQIARGMEYLESK | 121 |
| NOV1: | 307 | NYIHRDLAARNILVGENTLCKVGDFGLAR----------------LIKWTAPEALSRGHY | 350 |
| Tyrkc: | 122 | NFVHRDLAARNCLVGENKTVKIADFGLARDLYDDDYYRKKKSPRLPIRWMAPESLKDGKF | 181 |
| NOV1: | 351 | STKSDVWSFGILLHEMFSRGQVPYPGMSNHEAFLRVDAGYRMPCPLECPPSVHKLMLTCW | 410 |
| TyrKc: | 182 | TSKSDVWSFGVLLWEIFTLGESPYPGMSNEEVLEYLKKGYRLPQPPNCPDEIYDLMLQCW | 241 |
| NOV1: | 411 | CRDPEQRPCFKALRERL | 427 |
| TyrKc: | 242 | AEDPEDRPTFSELVERL | 258 |

TABLE 1F
Domain Analysis of NOV1

gnl|Pfam|pfam00069, pkinase, Protein kinase domain.

| (SEQ ID NO: 2) | | | |
|---|---|---|---|
| NOV1: | 191 | FTLCRKLGSGYFGEVFEGLWKDRVQ-VAIKVISRDNL-LHQQMLQSEIQAMKKLRHKHIL | 248 |
| (SEQ ID NO: 30) | | | |
| ProKi: | 1 | YELGEKLGSGAFGKVYKGKHKDTGEIVAIKILKKRSLSEKKKRFLREIQILRRLSHPNIV | 60 |
| NOV1: | 249 | ALYAVVSVGDPVYIITELMAKGSLLELLRDSDEKVLPVSELLDIAWQVAEGMCYLESQNY | 308 |
| ProKi: | 61 | RLLGVFEEDDHLYLVMEYMEGGDLFDYLRRNG-LLLSEKEAKKIALQILRGLEYLHSRGI | 119 |
| NOV1: | 309 | IHRDLAARNILVGENTLCKVGDFGLARLIK---------------WTAPEALSRGHYSTK | 353 |
| ProKi: | 120 | VHRDLKPENILLDENGTVKIADFGLARKLESSSYEKLTTFVGTPEYMAPEVLEGRGYSSK | 179 |
| NOV1: | 354 | SDVWSFGILLHEMFSRGQVPYPGMSNHEAFLRVDAG--YRMPCPLECPPSVHKLMLTCWC | 411 |
| ProKi: | 180 | VDVWSLGVILYELLT-GKLPFPGIDPLEELFRIKERPRLRLPLPPNCSEELKDLIKKCLN | 238 |
| NOV1: | 412 | RDPEQRPCFKALRERL 427 | |
| ProKi: | 239 | KDPEKRPTAKEILNHP 254 | |

TABLE 1G
Domain Analysis of NOV1

gnl|Smart|smart00220, S_TKc, Serine/Threonine protein kinases, catalytic
domain; Phosphotransferases. Serine or threonine-specific kinase subfamily

(SEQ ID NO: 2)

| | | | |
|---|---|---|---|
| NOV1: | 191 | FTLCRKLGSGYFGEVFEGLWKDRVQ-VAIKVISRDNLL--HQQMLQSEIQAMKKLRKKHI | 247 |

(SEQ ID NO: 31)

| | | | |
|---|---|---|---|
| S/ThKc: | 1 | YELLEVLGKGAFGKVYLARDKKTGKLVAIKVIKKEKLKKKKRERILREIKILKKLDHPNI | 60 |
| NOV1: | 248 | LALYAVVSVGDPVYIITELMAKGSLLELLRDSDEKVLPVSELLDIAWQVAEGMCYLESQN | 307 |
| S/ThKc: | 61 | VKLYDVFEDDDKLYLVMEYCEGGDLFDLLKK--RGRLSEDEARFYARQILSALEYLHSQG | 118 |
| NOV1: | 308 | YIHRDLAARNILVGENTLCKVGDFGLARLIK--------------WTAPEALSRGHYSTK | 353 |
| S/ThKc: | 119 | IIHRDLKPENILLDSDGHVKLADFGLAKQLDSGGTLLTTFVGTPEYMAPEVLLGKGYGKA | 178 |
| NOV1: | 354 | SDVWSFGILLHEMFSRGQVPYPGMSNHEAFLRV---DAGYRMPCPLECPPSVHKLMLTCW | 410 |
| S/ThKc: | 179 | VDIWSLGVILYELLT-GKPPFPGDDQLLALFKKIGKPPPPFPPPEWKISPEAKDLIKKLL | 237 |
| NOV1: | 411 | CRDPEQRPCFKAL | 423 |
| S/ThKc: | 238 | VKDPEKRLTAEEA | 250 |

There is strong evidence that tyrosine kinases are involved in the regulation of cellular growth and tumor progression. Over-expressions of tyrosine kinases have been documented in a number of neoplasms. Additionally, many retroviral and cellular oncogenes encode tyrosine kinase variants that are constitutively active. Recent evidence suggests that the intracellular targets of tyrosine kinases contain a protein module of approximately 100 amino acids, the Src homology 2 (SH2) domain. SH2 domains directly recognize tyrosine phosphorylation sites, and are thereby recruited to activated, autophosphorylated growth factor receptors. These interactions, in turn, stimulate the biochemical signalling pathways that control gene expression, cytoskeletal architecture, and cell metabolism. SH2-containing proteins frequently contain a distinct element of approximately 50 residues, the SH3 domain, that recognizes proline-rich motifs. Proteins with SH2 and SH3 domains can act as adaptors to couple tyrosine kinases to downstream targets with SH3-binding sites. A specific example of the synergistic action of SH2 and SH3 domains involves regulation of the Ras pathway by the adaptor protein Sem-5/drk/Grb2, which links tyrosine kinases to the Ras guanine nucleotide releasing protein Sos, which converts Ras to the active GTP-bound state.

Mitchell et al. (Oncogene 9:2383,1994) used a PCR-based differential screening procedure to isolate a novel protein-tyrosine kinase that they termed BRK for ‘breast tumor kinase.’ They found that the full-length cDNA, cloned from human breast tumor cell lines, was similar to other tumor-related kinases, particularly those of the SRC family. The encoded 451 -amino acid polypeptide sequence was composed of 3 domains: an SH3 domain, an SH2 domain, and a catalytic domain. The sequence of BRK, unlike that of SRC, does not include an N-terminal myristoylation domain. Using Northern blotting and RT-PCR, Mitchell et al. (1994) detected low levels of BRK transcripts in some breast tumor cell lines, but not in normal breast tissue or other tissues tested. It was also shown that BRK is capable of tyrosine autophosphorylation.

Kamalati et al. (J. Biol. Chem. 271:30956 1996) found that overexpression of BRK in mammary epithelial cells led to sensitization of cells to epidermal growth factor and resulted in a partially transformed phenotype. They also demonstrated coimmunoprecipitation of BRK and the EGF receptor. Mutational analysis suggested that while SRC and BRK share some functional properties, they function differently during transformation.

Recently, Derry et al. (Mol. Cel. Biol. 20:6114, 2000) have shown that Sik (mouse ortholog of BRK) and BRK to be the first identified tyrosine kinases that can phosphorylate Sam68 (an RNA binding protein) and regulate its activity within the nucleus, where it resides during most of the cell cycle. SAM68 is a tyrosine-phosphorylated, Src-associated protein in mitotic cells. Sam68 has been postulated to have a role in cell cycle control, particularly at the G1/S transition.

Like Sik, BRK is expressed in normal epithelial cells of the gastrointestinal tract that are undergoing terminal differentiation. BRK expression also increased during differentiation of the Caco-2 colon adenocarcinoma cell line. Modest increases in BRK expression were detected in primary colon tumors by RNase protection, in situ hybridization, and immunohistochemical assays. The BRK tyrosine kinase appears to play a role in signal transduction in the normal gastrointestinal tract, and its overexpression may be linked to the development of a variety of epithelial tumors.

BRK is also implicated in melanoma, since mRNA for the non-receptor kinase PTK6/BRK was not detected in normal melanocytes or primary melanoma lines, but was found in 9% of metastatic melanoma cell lines.

The disclosed NOV1 nucleic acid of the invention encoding a tyrosine protein kinase-6-like protein includes the nucleic acid whose sequence is provided in Table 1 A or a fragment thereof. The invention also includes a mutant or variant nucleic acid any of whose bases may be changed from the corresponding base shown in Table I while still encoding a protein that maintains its tyrosine protein kinase-6-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV1 protein of the invention includes the tyrosine protein kinase-6-like protein whose sequence is provided in Table 1B. The invention also includes a mutant or variant protein any of whose residues may be changed from the corresponding residue shown in Table 1B while still encoding a protein that maintains its tyrosine protein kinase-6 -like activities and physiological functions, or a functional fragment thereof. In the mutant or variant protein, up to about 60% percent of the residues may be so changed.

The invention further encompasses antibodies and antibody fragments, such as F_{ab} or (F_{ab})₂, that bind immunospecifically to any of the proteins of the invention.

The above defined information for this invention suggests that this tyrosine protein kinase-6-like protein (NOV1) may function as a member of a “tyrosine protein kinase-6 family”. Therefore, the NOV1 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV1 nucleic acids and proteins of the invention are useful in potential therapeutic applications implicated in cancer including but not limited to various pathologies and disorders as indicated below. For example, a cDNA encoding the tyrosine protein kinase-6-like protein (NOV1) may be useful in gene therapy, and the tyrosine protein kinase-6-like protein (NOV1) may be useful when administered to a subject in need thereof. By way of nonlimiting example, the compositions of the present invention will have efficacy for treatment of patients suffering from cancer, especially but not limited to, breast cancer, colorectal cancer and melanoma. The NOV1 nucleic acid encoding the tyrosine protein kinase-6-like protein of the invention, or fragments thereof, may further be useful in diagnostic applications, wherein the presence or amount of the nucleic acid or the protein are to be assessed.

NOV1 nucleic acids and polypeptides are further useful in the generation of antibodies that bind immuno-specifically to the novel NOV1 substances for use in therapeutic or diagnostic methods. These antibodies may be generated according to methods known in the art, using prediction from hydrophobicity charts, as described in the “Anti-NOVX Antibodies” section below. The disclosed NOV1 protein has multiple hydrophilic regions, each of which can be used as an immunogen. In one embodiment, a contemplated NOV1 epitope is from about amino acids 40 to 60. In another embodiment, a NOV1 epitope is from about amino acids 130 to 150. In additional embodiments, NOV1 epitopes are from about amino acids 240 to 270, from about amino acids 280 to 300, from about amino acid 310 to 340, and from about amino acids 350 to 370. These novel proteins can be used in assay systems for functional analysis of various human disorders, which will help in understanding of pathology of the disease and development of new drug targets for various disorders.

NOV2

NOV2 includes four novel Keratin 4-like proteins disclosed below. The disclosed sequences have been named NOV2a, NOV2b, NOV2c and NOV2d. NOV2 is localized to human chromosome 17.

NOV2a

A disclosed NOV2a nucleic acid of 1682 nucleotides (also referred to as AC058790_da1) encoding a novel Keratin 4-like protein is shown in Table 2A. An open reading frame was identified beginning with an ATG initiation codon at nucleotides 54-56 and ending with a TGA codon at nucleotides 1680-1682. A putative untranslated region upstream from the initiation codon is underlined in Table 2A. The start and stop codons are in bold letters.

TABLE 2A
NOV2a nucleotide sequence.

(SEQ ID NO:3)

TCCTGCAGGTCCCTGTCACTTCTCTGATAGCTCCCAGCTCGCTCTCTGCAGCCATGATTGCCAGACAGCA
GTGTGTCCGAGGCGGGCCCCGGGGCTTCAGCTGTGACTCGGCCATTGTAGGCGGTGGCAAGAGAGGTGCC
TTCAGCTCAGTCTCCATGTCTGGAGGTGCTGGCCGATGCTCTTCTGGGGAGATTTGGCAGCAGAAGCCTCT
ACAACCTCAGGGGGAACAAAAGCATCTCCATGAGTGTGGCTAGGTCACGACAAGGTGCCTGCTTTGGGG
TGCTGGAGGCTTTGGCACTGGTGGCTTTGGTGGTGGATTTGGGGGCTCCTTCAGTGGTAAGGGTGGCCCT
GGCTTCCCCGTCTGCCCCGCTGGGGGAATTCAGGAGGTCACCATCAACCAGAGCTTGCTCACCCCCCTCC
ACGTGGAGATTGACCCTGAGATCCAGAAAGTCCGGACGGAAGAGCGCGAACAGATCAAGCTCCTCAACAA
CAAGTTTGCCTCCTTCATCGAGCAGGTGCGGTTCCTGGAGCAACAGAATAAGGTGCTGGAGACCAAGTGG
GCACTGCTGCAGGAGCAGGGCCAGAACTTGGGTGTCACCAGGAACAACCTGGAGCCCCTCTTTGAGGCCT
ACCTGGGTAGCATGCGGAGCACGCTGGACAGACTTCAGAGCGAGCGGGGGAGGCTGGACTCAGAGCTCAG
GAACGTGCAGGACCTTGTGGAGGACTTCAAGAACAAGTATGAAGAGGAGATCAACAAACGCACAGCAGCC
GAGAATGACTTTGTGGTCCTAAAGAAGTATGAGACAGAGCTGGCCATGCGCCAGTCTGTGGAGAACGACA
TCCATGGGCTCCGCAAGGTCATTGATGACACCAATATCACACGACTGCAGCTGGAGACAGAGATCGAGGC
TCTCAAGGAGGAGCTGCTCTTCATGAAGAAGAACCACGAAGAGGAGCTGGGCCAGCTCCAGACCCAGGCC
AGCGACACGTCTGTGGTGCTGTCCATGGACAACAACCGCTACCTGGACTTCAGCAGCATCATCACTGAGG
TCCGCGCCCGGTACGAGGAGATCGCCCGGAGCAGCAAGGCTGAGGCTGAGGCCTTGTACCAGACCAAGGT
GCAGGAACTTCAGGTGTCTGCCCAGCTTCATGGGGACAGGATGCAGGAAACGAAAGTCCAGATCTCTCAG
CTACACCAAGAGATTCAGAGGCTGCAGAGTCAGACTGAGAACCTCAAGAAGCAGAGGGCTTCCCTGGAGG
CCGCCATTGCAGATGCCGAGAGCGTGGAGAGCTGGCCATTAAGGATGCCAACGCCAAGTTGTCCGAGCT
GGAGGCCGCCCTGCAGCGGGCCAAGCAGGACATGGCGCGGCAGCTGCGTGAGTACCAGGAGCTGATGAA
GTCAAGCTGGCCCTGGACATCGAGATCGCCACCTACAGGAAGCTGCTGGAGGGCGAGGAGAGCCGGATGT
CTGGAGAATGCCAGAGTGCCGTGAGCATCGCTGTGGTCAGCGGTAGCACCAGCACTGGAGGCATCAGCGG
AGGATTAGGAAGTGGCTCCGGGTTTGGCCTGAGTAGTGGCTTTGGCTCCGGCTCTGGAAGTGGCTTTGGG
TTTGGTGGCAGTGTCTCTGGCAGTTCCAGCAGCAAGATCATCTCTACCACCACCCTGAACAAGAGACGATGA

In a search of sequence databases, it was found, for example, that a NOV2a nucleic acid sequence has 893 of 1207 bases (73%) identical to a 1746 bp cytokeratin 4 C-terminal region mRNA from Homo sapiens (GENBANK-ID: HSKERC4|acc:X07695. Public nucleotide databases include all GenBank databases and the GeneSeq patent database.

The disclosed NOV2a polypeptide (SEQ ID NO: 4) encoded by SEQ ID NO: 3 has 542 amino acid residues and is presented in Table 2B using the one-letter amino acid code. Signal P, Psort and/or Hydropathy results predict that NOV2a does not have a signal peptide and is likely to be localized in the cytoplasm a certainty of 0. 4500. In other embodiments, NOV2a may also be localized to the microbody with a certainty of 0.3000, the mitochondrial matrix with a certainty of 0.1000, or in the lysosome with a certainty of 0.1000.

Exon linking data for NOV2 can be found below in Example 1. SNP data for NOV2 can be found below in Example 3.

TABLE 2B
Encoded NOV2a protein sequence.

(SEQ ID NO:4)

MIARQQCVRGGPRGFSCDSAIVGGGKRGAFSSVSMSGGAGRCSSGGFGSRSLYNLRGNKSISMSVARSRQ
GACFGGAGGFGTGGFGGGFGGSFSGKGGPGFPVCPAGGIQEVTINQSLLTPLHVEIDPEIQKVRTEEREQ
IKLLNNKFASFIEQVRFLEQQNKVLETKWALLQEQGQNLGVTRNNLEPLFEAYLGSMRSTLDRLQSERGR
LDSELRNVQDLVEDFKNKYEEEINKRTAAENDFVVLKKYETELAMRQSVENDIHGLRKVIDDTNITRLQL
ETEIEALKEELLFMKKNHEEELGQLQTQASDTSVVLSMDNNRYLDFSSIITEVRARYEEIARSSKAEAEA
LYQTKVQELQVSAQLHGDRMQETKVQISQLHQEIQRLQSQTENLKKQRASLEAAIADAEQRGELAIKDAN
AKLSELEAALQRAKQDMARQLREYQELMNVKLALDIEIATYRKLLEGEESRMSGECQSAVSIAVVSGSTS
TGGISGGLGSGSGFGLSSGFGSGSGSGFGFGGSVSGSSSSKIISTTTLNKRR

The full amino acid sequence of the disclosed NOV2a protein was found to have 331 of 542 amino acid residues (61%) identical to, and 401 of 542 amino acid residues (73%) similar to, the 543 amino acid residue keratin 4, type II, cytoskeletal protein from Homo sapiens (Human) (pir-id:I37942). Public amino acid databases include the GenBank databases, SwissProt, PDB and PIR.

NOV2b

A disclosed NOV2b nucleic acid of 1625 nucleotides (also referred to as AC058790_da2) encoding a novel Keratin 4-like protein is shown in Table 2C. An open reading frame was identified beginning with an ATG initiation codon at nucleotides 54-56 and ending with a TGA codon at nucleotides 1623-1625. A putative untranslated region upstream from the initiation codon is underlined in Table 2C. The start and stop codons are in bold letters.

TABLE 2C
NOV2b nucleotide sequence.

(SEQ ID NO:5)

TCCTGCAGGTCCCTGTCACTTCTCTGATAGCTCCCAGCTCGCTCTCTGCAGCCATGATTGCCAGACAGCA
GTGTGTCCGAGGCGGGCCCCGGGGCTTCAGCTGTGACTCGGCCATTGTAGGCGGTGGCAAGAGAGGTGCC
TTCAGCTCAGTCTCCATGTCTGGAGGTGCTGGCCGATGCTCTTCTGGGGGATTTGGCAGCAGAAGCCTCT
ACAACCTCAGGGGGAACAAAAGCATCTCCATGAGTGTGGCTAGGTCACGACAAGGTGCCTGCTTTGGGGG
TGCTGGAGGCTTTGGCACTGGTGGCTTTGGTGGTGGATTTGGGGGCTCCTTCAGTGGTAAGGGTGGCCCT
GGCTTCCCCGTCTGCCCCGCTGGGGGAATTCAGGAGGTCACCATCAACCAGAGCTTGCTCACCCCCCTCC
ACGTGGAGATTGACCCTGAGATCCAGAAAGTCCGGACGGAAGAGCGCGAACAGATCAAGCTCCTCAACAA
CAAGTTTGCCTCCTTCATCGAGCAGGTGCGGTTCCTGGAGCAACAGAACAAAGTCCTGGAGACCAAGTGG
AACCTGCTCCAGCAGCAGGGCACAAGTTCCATCTCAGGCACAAACAACCTTGAGCCTCTTTTTGAGAATC
ACATCAACTACCTGCGGAGCTACCTGGACAACATCCTCGGGGAGAGAGGGCGCCTGGACTCTGAGCTGAA
GAACATGGAGGACCTGGTGGAAGACTTCAAGAAGAAGTATGAGGATGAAATCAATAAACGTACAGCTGCT
GAGAATGAATTTGTGACTCTGAAGAAGGATGTGGACAGTGCCTATATGAACAAGGTGGAGCTTCAGGCCA
AAGTGGATGCCTTGATAGATGAGATCGACTTCTTAAGGACCCTCTACGACGCTGAGCTGAGCCAAGTGCA
GACCCACGTGTCTAACACCAATGTGGTGCTGTCCATGGACAACAACCGCAACCTGGACCTGGACAGCATC
ATCGCCGAGGTCAAGGCCCAGTATGAGCTGATTGCCCAGAGGAGCCGGGCTGAGGCCGAGGCCTGGTACC
AGACCAAGGTGGAGGAGCTGCAGGTGACTGCTGGGAAGCATGGGGACAACCTGCGGGACACCAAGAACGA
GATTGCTGAGCTCACCCGCACTATCCAGAGGCTGCAGGGGGAGGCTGATGCAGCCAAGAAGCAGCAGTGT
CAGCAGCTGCAGACGGCCATTGCGGAAGCGGAGCAGCGTGGGGAGCTGGCACTCAAGGATGCTCAGAAGA
AGCTTGGGGATCTGGATGTGGCCCTGCACCAGGCCAAGGAGGACCTGACACGGCTGCTGCGTGACTACCA
GGAGCTGATGAATGTCAAGCTGGCCCTGGACGTGGAGATTGCCACCTACCGCAAGCTTCTGGAGAGCCAG
GAGAGCAGGATGTCTGGAGAATGCCAGAGTGCCGTGAGCATCGCTGTGGTCAGCGGTAGCACCAGCACTG
GAGGCATCAGCGGAGGATTAGGAAGTGGCTCCGGGTTTGGCCTGAGTAGTGGCTTTGGCTCCGGCTCTGG
AAGTGGCTTTGGGTTTGGTGGCAGTGTCTCTGGCAGTTCCAGCAGCAAGATCATCTCTACCACCACCCTG
AACAAGAGACGATGA

In a search of sequence databases, it was found, for example, that a NOV2b nucleic acid sequence has 1018 of 1355 bases (75%) identical to a a 2181 bp type II 57 kd keratin mRNA from Mus musculus (GENBANK-ID: MMKER57R|acc:X03491. Public nucleotide databases include all GenBank databases and the GeneSeq patent database.

The disclosed NOV2b polypeptide (SEQ ID NO: 6) encoded by SEQ ID NO: 5 has 523 amino acid residues and is presented in Table 2D using the one-letter amino acid code. Signal P, Psort and/or Hydropathy results predict that NOV2b does not have a signal peptide and is likely to be localized in the cytoplasm a certainty of 0.6500. In other embodiments, NOV2b may be in the mitochondrial matrix with a certainty of 0.1000, or in the lysosome with a certainty of 0.1000.

TABLE 2D
Encoded NOV2b protein sequence.

(SEQ ID NO:6)

MIARQQCVRGGPRGFSCDSAIVGGGKRGAFSSVSMSGGAGRCSSGGFGSRSLYNLRGNKSISMSVARSRQ
GACFGGAGGFGTGGFGGGFGGSFSGKGGPGFPVCPAGGIQEVTINQSLLTPLHVEIDPEIQKVRTEEREQ
IKLLNNKFASFIEQVRFLEQQNKVLETKWNLLQQQGTSSISGTNNLEPLFENHINYLRSYLDNILGERGR
LDSELKNMEDLVEDFKKKYEDEINKRTAAENEFVTLKKDVDSAYMNKVELQAKVDALIDEIDFLRTLYDA
ELSQVQTHVSNTNVVLSMDNNRNLDLDSIIAEVKAQYELIAQRSRAEAEAWYQTKVEELQVTAGKHGDNL
RDTKNEIAELTRTIQRLQGEADAAKKQQCQQLQTAIAEAEQRGELALKDAQKKLGDLDVALHQAKEDLTR
LLRDYQELMNVKLALDVEIATYRKLLESQESRMSGECQSAVSIAVVSGSTSTGGISGGLGSGSGFGLSSG
FGSGSGSGFGFGGSVSGSSSSKIISTTTLNKRR

The full amino acid sequence of the disclosed NOV2b protein was found to have 430 of 534 amino acid residues (80%) identical to, and 478 of 534 amino acid residues (89%) similar to, the 543 amino acid residue keratin 4, type II, cytoskeletal protein from Homo sapiens (Human) (pir-id:I37942). Public amino acid databases include the GenBank databases, SwissProt, PDB and PIR.

NOV2c

A disclosed NOV2c nucleic acid of 1619 nucleotides (also referred to as AC058790_da3) encoding a novel Keratin 4-like protein is shown in Table 2E. An open reading frame was identified beginning with an ATG initiation codon at nucleotides 54-56 and ending with a TGA codon at nucleotides 1617-1619. A putative untranslated region upstream from the initiation codon is underlined in Table 2E. The start and stop codons are in bold letters.

TABLE 2E
NOV2c nucleotide sequence.

| | |
|---|---|
| TCCTGCAGCTCCCTGTCACTTCTCTGATAGCTCCCAGCTCGCTCTCTGCAGCCATGATTGCCAGACAGCA | (SEQ ID NO:7) |
| GTGTGTCCGAGGCGGGCCCCGGGGCTTCAGCTGTGACTCGGCCATTGTAGGCGGTGGCAAGAGAGGTGCC | |
| TTCAGCTCAGTCTCCATGTCTGGAGGTGCTGGCCGATGCTCTTCTGGGGGATTTGGCAGCAGAAGCCTCT | |
| ACAACCTCAGGGGGAACAAAAGCATCTCCATGAGTGTGGCTAGGTCACGACAAGGTGCCTGCTTTGGGGG | |
| TGCTGGAGGCTTTGGCACTGGTGGCTTTGGTGGTGGATTTGGGGGCTCCTTCAGTGGTAAGGGTGGCCCT | |
| GGCTTCCCCGTCTGCCCCGCTGGGGGAATTCAGGAGGTCACCATCAACCAGAGCTTGCTCACCCCCCTCC | |
| ACGTGGAGATTGACCCTGAGATCCAGAAAGTCCGGACGGAAGAGCGCGAACACATCAAGCTCCTCAACAA | |
| CAAGTTTGCCTCCTTCATCGAGCAGGTGCGGTTCCTGGAGCAGCAGAACAAGGTCCTGGAGACGAAGTGG | |
| CATCTGCTGCAGCAACAGGGGTTGAGTGGCAGCCAGCAGGGCCTGGAGCCTGTCTTTGAGGCCTGCCTGG | |
| ATCAGCTCAGGAGCAGCTGGAGCAGCTCCAGGGAGAACGAGGGGCTCTGGATGCTGAGTTGAAGGCCTGG | |
| CCGGGACCAGGAGGAGGAGTATAAGTCCAAGTATGAGGATGAGATCAATAAGCGTACAGAGATGGAGAAC | |
| GAATTTGTCCTCATCAAGAAGGATGTGGATGAAGCTTACATGAACAAGGTAGAGCTGGAGTCTCGCCTGG | |
| AAGGGCTGACCGACGAGATCAACTTCCTCAGGCAGCTATATGAAGAGGAGATCCGGGAGCTGCAGTCCCA | |
| GATCTCGGACACATCTGTGGTGCTGTCCATGGACAACAGCCGCTCCCTGGACATGGACAGCATCATTGCT | |
| GAGGTCAAGGCACAGTACGAGGATATTGCCAACCGCAGCCGGGCTGAGGCTGAGAGCATGTACCAGATCA | |
| AGTATGAGGAGCTGCAGAGCCTGGCTGGGAAGCACGGGGATGACCTGCGGCGCACAAAGACTGAGATCTC | |
| TGAGATGAACCGGAACATCAGCCGGCTCCAGGCTGAGATTGAGGGCCTCAAAGGCCAGAAGGCCAGCTTG | |
| GAGAACAGCCTGAGGGAGGTGGAGGCCCGCTACGCCCTACAGATGGAGCAGCTCAACGGGATCCTGCTGC | |
| ACCTTGAGTCAGAGCTGGCACAGACCCGGGCAGAGGGACAGCGCCAGGCCCAGGAGTATGAGGCCCTGCT | |
| GAACATCAAGGTCAAGCTGGAGGCTGAGATCGCCACCTACCGCCGCCTGCTGGAAGATGGCGAGGACTTT | |
| AAGATGTCTGGAGAATGCCAGAGTGCCGTGAGCATCGCTGTGGTCAGCGGTAGCACCAGCACTGGAGGCA | |
| TCAGCGGAGGATTAGGAAGTGGCTCCGGGTTTGGCCTGAGTAGTGGCTTTGGCTCCGGCTCTGGAAGTGG | |
| CTTTGGGTTTGGTGGCAGTGTCTCTGGCAGTTCCAGCAGCAAGATCATCTCTACCACCACCCTGAACAAG | |
| AGACGATGA | |

In a search of sequence databases, it was found, for example, that a NOV2c nucleic acid sequence has 949 of 1355 bases (70%) identical to a 2181 bp type II 57 kd keratin mRNA from Mus musculus (GENBANK-ID: MMKER57R|acc:X03491). Public nucleotide databases include all GenBank databases and the GeneSeq patent database.

The disclosed NOV2c polypeptide (SEQ ID NO: 8) encoded by SEQ ID NO: 7 has 521 amino acid residues and is presented in Table 2F using the one-letter amino acid code. Signal P, Psort and/or Hydropathy results predict that NOV2c does not have a signal peptide and is likely to be localized in the cytoplasm a certainty of 0.4500. In other embodiments, NOV2c may be in the microbody with a certainty of 0.3000, in the microchondrial matrix space with a certainty of 0.100, or in the lysosome with a certainty of 0.1000.

TABLE 2F
Encoded NOV2c protein sequence.

| | |
|---|---|
| MIARQQCVRGGPRGFSCDSAIVGGGKRGAFSSVSMSGGAGRCSSGGFGSRSLYNLRGNKSISMSVARSRQ | (SEQ ID NO:8) |
| GACFGGAGGFGTGGFGGGFGGSFSGKGGPGFPVCPAGGIQEVTINQSLLTPLHVEIDPEIQKVRTEEREQ | |
| IKLLNNKFASFIEQVRFLEQQNKVLETKWHLLQQQGLSGSQQGLEPVFEACLDQLRKQLEQLQGERGALD | |
| AELKACRDQEEEYKSKYEDEINKRTEMENEFVLIKKDVDEAYMNKVELESRLEGLTDEINFLRQLYEEEI | |
| RELQSQISDTSVVLSMDNSRSLDMDSIIAEVKAQYEDIANRSRAEAESMYQIKYEELQSLAGKHGDDLRR | |
| TKTEISEMNRNISRLQAEIEGLKGQKASLENSLREVEARYALQMEQLNGILLHLESELAQTRAEGQRQAQ | |
| EYEALLNIKVKLEAEIATYRRLLEDGEDFKMSGECQSAVSIAVVSGSTSTGGISGGLGSGSGFGLSSGFG | |
| SGSGSGFGFGGSVSGSSSSKIISTTTLNKRR | |

The full amino acid sequence of the disclosed NOV2c protein was found to have 379 of 534 amino acid residues (70%) identical to, and 451 of 534 amino acid residues (84%) similar to, the 534 amino acid residue keratin 4, type II, cytoskeletal protein from Homo sapiens (pir-id:I37942). Public amino acid databases include the GenBank databases, SwissProt, PDB and PIR.

TaqMan expression data for NOV2c is shown below in Example 2.

NOV2d

A disclosed NOV2d nucleic acid of 1619 nucleotides (also referred to as AC058790_da4) encoding a novel Keratin 4-like protein is shown in Table 2G. An open reading frame was identified beginning with an ATG initiation codon at nucleotides 54-56 and ending with a TGA codon at nucleotides 1617-1619. A putative untranslated region upstream from the initiation codon is underlined in Table 2G. The start and stop codons are in bold letters.

TABLE 2G
NOV2d nucleotide sequence.

| | |
|---|---|
| TCCTGCAGGTCCCTGTCACTTCTCTGATAGCTCCCAGCTCGCTCTCTGCAGCCATGATTGCCAGACAGCA | (SEQ ID NO:9) |
| GTGTGTCCGAGGCGGGCCCCGGGGCTTCAGCTGTGACTCGGCCATTGTAGGCGGTGGCAAGAGAGGTGCC | |
| TTCAGCTCAGTCTCCATGTCTGGAGGTGCTGGCCGATGCTCTTCTGGGGGATTTGGCAGCAGAAGCCTCT | |
| ACAACCTCAGGGGGAACAAAAGCATCTCCATGAGTGTGGCTAGGTCACGACAAGGTGCCTGCTTTGGGGG | |
| TGCTGGAGGCTTTGGCACTGGTGGCTTTGGTGGTGGATTTGGGGGCTCCTTCAGTGGTAAGGGTGGCCCT | |
| GGCTTCCCCGTCTGCCCCGCTGGGGGAATTCAGGAGGTCACCATCAACCAGAGCTTGCTCACCCCCCTCC | |
| ACGTGGAGATTGACCCTGAGATCCAGAAAGTCCGGACGGAAGAGCGCGAACAGATCAAGCTCCTCAACAA | |
| CAAGTTTGCCTCCTTCATCGAGCAGGTGCAGTTCTTAGAGCAACAGAATAAGGTCCTGGAGACCAAATGG | |
| AACCTGCTCCAGCAGCAGACGACCACCACCTCCAGCAAAAACCTTGAGCCCCTCTTTGAGACCTACCTCA | |
| GTGTCCTGAGGAAGCAGCTAGATACCTTGGGCAATGACAAAGGGCGCCTGCAGTCTGAGCTGAAGACCAT | |
| GCAGGACAGCGTGGAGGACTTCAAGACTAAGTATGAGGAGGAGGCCCACAGGCGTGCCACACTTGAGAAC | |
| GACTTTGTGGTCCTCAAGAAGGATGTGGATGGGGTTTTCCTGAGCAAGATGGAGTTGGAGGGCAAGCTGG | |
| AGGCTCTGAGAGAGTACCTCTACTTCTTGAAGCATCTGAATGAAGAAGTGGAGCTGTCCCAGATGCAGAC | |
| CCATGTCAGCGACACGTCCGTGGTCCTTTCCATGGACAACAACCGCAACCTGGACCTGGACAGCATTATT | |
| GCCGAGGTCCGTGCCCAGTACGAGGAGATTGCCCAGAGGAGCAAGGCTGAGGCTGAAGCCCTGTACCAGA | |
| CCAAGGTGCAGCAGCTCCAGATCTCGGTTGACCAACATGGTGACAACCTGAAGAACACCAAGAGTGAAAT | |
| TGCAGAGCTCAACAGGATGATCCAGAGGCTGCGGGCAGAGATCGAGAACATCAAGAAGCAGTGCCAGACT | |
| CTTCAGGTATCCGTGGCTGATGCAGAGCAGCGAGGTGAGAATGCCCTTAAAGATGCCCACAGCAAGCGCG | |
| TAGAGCTGGAGGCTGCCCTGCAGCAGGCCAAGGAGGAGCTGGCACGAATGCTGCGTGAGTACCAGGAGCT | |
| CATGAGTGTGAAGCTGGCCTTGGACATCGAGATCGCCACCTACCGCAAACTGCTGGAGGGCGAGGAGTAC | |
| AGGATGTCTGGAGAATGCCAGAGTGCCGTGAGCATCGCTGTGGTCAGCGGTAGCACCAGCACTGGAGGCA | |
| TCAGCGGAGGATTAGGAAGTGGCTCCGGGTTTGGCCTGAGTAGTGGCTTTGGCTCCGGCTCTGGAAGTGG | |
| CTTTGGGTTTGGTGGCAGTGTCTCTGGCAGTTCCAGCAGCAAGATCATCTCTACCACCACCCTGAACAAG | |
| AGACGATGA | |

In a search of sequence databases, it was found, for example, that a NOV2d nucleic acid sequence has 1171 of 1223 bases (95%) identical to a 1746 bp cytokeratin 4 C-terminal region mRNA from Homo sapiens (GENBANK-ID: HSKERC4|acc:X07695). Public nucleotide databases include all GenBank databases and the GeneSeq patent database.

The disclosed NOV2d polypeptide (SEQ ID NO: 10) encoded by SEQ ID NO: 9 has 521 amino acid residues and is presented in Table 2h using the one-letter amino acid code. Signal P, Psort and/or Hydropathy results predict that NOV2d does not have a signal peptide and is likely to be localized in the cytoplasm a certainty of 0.6500. In other embodiments, NOV2d may be in the microchondrial matrix space with a certainty of 0.100, or in the lysosome with a certainty of 0.1000.

TABLE 2H
Encoded NOV2d protein sequence.

| | |
|---|---|
| MIARQQCVRGGPRGFSCDSAIVGGGKRGAFSSVSMSGGAGRCSSGGFGSRSLYNLRGNKSISMSVARSRQ | (SEQ ID NO:10) |
| GACFGGAGGFGTGGFGGGFGGSFSGKGGPGFPVCPAGGIQEVTINQSLLTPLHVEIDPEIQKVRTEEREQ | |
| IKLLNNKFASFIEQVQFLEQQNKVLETKWNLLQQQTTTTSSKNLEPLFETYLSVLRKQLDTLGNDKGRLQ | |
| SELKTMQDSVEDFKTKYEEEAHRRATLENDFVVLKKDVDGVFLSKMELEGKLEALREYLYFLKHLNEEVE | |
| LSQMQTHVSDTSVVLSMDNNRNLDLDSIIAEVRAQYEEIAQRSKAEAEALYQTKVQQLQISVDQHGDNLK | |
| NTKSEIAELNRMIQRLRAEIENIKKQCQTLQVSVADAEQRGENALKDAHSKRVELEAALQQAKEELARML | |
| REYQELMSVKLALDIEIATYRKLLEGEEYRMSGECQSAVSIAVVSGSTSTGGISGGLGSGSGFGLSSGFG | |
| SGSGSGFGFGGSVSGSSSSKIISTTTLNKRR | |

The full amino acid sequence of the disclosed NOV2d protein was found to have 489 of 534 amino acid residues (91%) identical to, and 504 of 534 amino acid residues (94%) similar to, the 534 amino acid residue keratin 4, type II, cytoskeletal protein from Homo sapiens (pir-id:I37942). Public amino acid databases include the GenBank databases, SwissProt, PDB and PIR.

TaqMan expression data for NOV2d is shown below in Example 2.

The proteins encoded by the NOV2a, 2b, 2c and 2d nucleotides are very closely homologous as is shown in the alignment in Table 2I.

Homologies to any of the above NOV2 proteins will be shared by the other three NOV2 proteins insofar as they are homologous to each other as shown above. Any reference to NOV2 is assumed to refer to all four of the NOV2 proteins in general, unless otherwise noted.

The disclosed NOV2 polypeptide has homology to the amino acid sequences shown in the BLASTP data listed in Table 2J.

TABLE 1J
BLAST results for NOV2

| Gene Index/ | | Length | Identity | Positives | |
|---|---|---|---|---|---|
| Identifier | Protein/Organism | (aa) | (%) | (%) | Expect |
| gi\|11276929\| | Type II keratin- | 534 | 72 | 81 | e-164 |
| | 4/human | | | | |
| gi\|547753\| | Type II keratin- | 534 | 72 | 81 | e-164 |
| | 4/human | | | | |
| gi\|6678645\| | Keratin complex | 524 | 64 | 76 | e-146 |
| | 2/mouse | | | | |
| gi\|7161776 | Cytokeratin/ | 551 | 59 | 73 | e-137 |
| | human | | | | |
| gi\|4758618\| | Cytokeratin | 551 | 59 | 72 | e-136 |
| | type II/human | | | | |

The homology between these and other sequences is shown graphically in the ClustalW analysis shown in Table 2K.

[image]

[image]

[image]

DOMAIN results for NOV2 as disclosed in Tables 2M-N, were collected from the Conserved Domain Database (CDD) with Reverse Position Specific BLAST analyses. This indicates that the NOV2 sequence has properties similar to those of other proteins known to contain this domain.

TABLE 2M
Domain Analysis of NOV2

gnl|Pfam|pfam00038, filament, Intermediate filament protein.

| | | | | |
|---|---|---|---|---|
| NOV2 | 136 | EEREQIKLLNNKFASFIEQVRFLEQQNKVLETKWALLQEQGQNLGVTRNNLEPLFEAYLG | 195 | (SEQ ID NO: 4) |
| InFil: | 1 | NEKEQMQNLNDRLASYIDKVRFLEQQNKELEVKIEELRQKQAPSVS---RLYSLYETEIE | 57 | (SEQ ID NO: 32) |
| NOV2 | 196 | SMRSTLDRLQSERGRLDSELRNVQDLVEDFKNKYEEEINKRTAAENDFVVLKKYETELAM | 255 | |
| InFil: | 56 | ELRRQIDQLTNERARLQLEIDNLREAAEDFRKKYEDEINLR------------------- | 98 | |
| NOV2 | 256 | RQSVENDIHGLRKVIDDTNITRLQLETEIEALKEELLFMKKNHEEELGQLQTQASDTSVV | 315 | |
| InFil: | 99 | -QEAENDLVGLRKDLDEATLARVDLENKVESLQEELEFLKKNHEEEVKELQAQIQDTVNV | 157 | |
| NOV2 | 316 | LSMDNNRYLDFSSIITEVRARYEEIARSSKAEAEALYQTKVQELQVSAQLHGDRMQETKV | 375 | |
| InFil: | 158 | -EMDAARKLDLTKALREIRAQYEEIAKKNRQEAEEWYKSKLEELQTAAARNGEALRSAKE | 216 | |
| NOV2 | 376 | QISQLHQEIQRLQSQTENLKKQRASLEAAIADAEQRGELAIKDANAKLSELEAALQRAKQ | 435 | |
| InFil: | 217 | EITELRRQIQSLEIELQSLKAQNASLERQLAELEERYELELRQYQALISQLEEELQQLRE | 276 | |
| NOV2 | 436 | DMARQLREYQELMNVKLALDIEIATYRKLLEGEESR 471 | | |
| InFil: | 277 | EMARQLREYQELLDVKLALDIEIATYRKLLEGEESR 312 | | |

TABLE 2N
Domain Analysis of NOV2

gnl|Pfam|pfam01576, Myosin_tail, Myosin tail (Myosn).

| | | | | |
|---|---|---|---|---|
| NOV2: | 131 | QKVRTEEREQIKLLNNKFASFIEQVRFLEQQNKVLETKWALLQEQGQNLGVTRNNLEPL- | 189 | (SEQ ID NO: 4) |
| Myosn: | 179 | EKKAKQLESQLSELQVKLDELQRQLNDLTSQKSRLQSENSDLTRQLEEAEAQVSNLSKLK | 238 | (SEQ ID NO: 33) |
| NOV2: | 190 | --FEAYLGSMRSTLDRLQSERGRLDSELRNVQDLVEDFKNKYEEEINKRTAAENDFVVLK | 247 | |
| Myosn: | 239 | SQLESQLEEAKRSLEEESRERANLQAQLRQLEHDLDSLREQLEEESEAKAELERQLSKAN | 298 | |
| NOV2: | 248 | KYETELAMRQSVENDIHGLRKVIDDTNITRLQLETEIEALKEELLFMKKNHEEELGQLQT | 307 | |
| Myosn: | 299 | AEIQQWRSKFESEGALR--AEELEELKKKLNQKISELEEAAEAANAKCDSLEKTKSRLQS | 386 | |
| NOV2: | 308 | QASDTSVVLSMDNN-------RYLDFSSIITEVRARYEEIA--RSSKAEAEALYQTKVQE | 358 | |
| Myosn: | 357 | ELEDLQIELERANAAASELEKKQKNFDKILAEWKRKVDELQAELDTAQREARNLSTELFR | 416 | |
| NOV2: | 359 | LQVSAQLHGDRMQETKVQISQLHQEIQRLQSQ----------TENLKKQRASLEAAIADA | 408 | |
| Myosn: | 417 | LKNELEELKDQVEALRRENKNLQDEIHDLTDQLGEGGRNVHELEKARRRLEAEKDELQAA | 476 | |
| NOV2: | 409 | EQRGELAIKDANAKLSELEAALQRAKQDMARQLREYQE 446 | | |
| Myosn: | 477 | LEEAEAALELEESKVLRAQVELSQIRSEIERRLAEKEE 514 | | |

Intermediate filaments are protein polymers which, together with actin filaments and microtubules, form the cytoskeleton of cells. In epithelial cells the intermediate filaments are made up of keratins, a large family of related polypeptides whose patterns of expression vary with cell type as well as with stage of epithelial differentiation. The more than 20 different keratins encoded by at least as many differentially expressed genes in humans (see review by Fuchs, 1988) can be subdivided into 2 classes: type I keratins (K10-K19) are small (40-56.5 kD) and relatively acidic (pI=4.5-5.5), whereas type II keratins (K1-K9) are larger (53-68 kD) and more basic (pI=5.5-7.5). Filament formation usually requires expression of keratins in pairs consisting of 1 type I and 1 type II polypeptide. The mspecific keratins expressed characterize the type of epithelial differentiation. For example, K5 (148040) and K14 (148066) are synthesized in the basal cell layer of all stratified squamous epithelia, while, in the course of stratification, terminally differentiating epidermal cells express K1 (139350) and K10 (148080), and suprabasal cells (i.e., maturing cells of nonkeratinizing squamous epithelia) express K4 and K13 (148065). Using SDS-PAGE, Mischke et al. (1990) identified 2 electrophoretic variants of the human keratin K4 that are expressed in squamous nonkeratinizing epithelia lining the upper digestive tract. Based on a large population sample, they concluded that 2 codominant alleles, a and b, are in Hardy-Weinberg equilibrium and studies in 2 families confirmed the mendelian nature of the variation. They referred to polymorphism also of the K1 and K10 keratins of human epidermis (Mischke and Wild, 1987).

Using a cDNA probe in the analysis of human-hamster cell hybrid DNAs, Romano et al. (1987, 1988) mapped the cytokeratin-4 gene to chromosome 12. Barletta et al. (1989, 1990) used in situ hybridization to demonstrate that the cytokeratin-4 gene localizes to human chromosome 12p11.2-q11.

In affected members of 2 Scottish kindreds with white sponge nevus (193900), Rugg et al. (1995) used RT-PCR followed by direct sequencing of K4 to demonstrate heterozygosity for a 3-bp deletion in the 1A domain. This deletion occurred in a (CAA) 3 repeat and resulted in deletion of amino acid asparagine-8 in the highly conserved helix initiation motif. This residue is conserved between all type I, type II, and type III intermediate filament proteins and is therefore predicted to be highly important to keratin filament assembly and/or integrity. Affected members of the 2 kindreds were found to share a common haplotype with 2 polymorphic flanking markers known to be physically very close to K4 and so these families were probably related. A full report of these findings appeared in Rugg et al. (1995); the authors noted that this mutation in KRT4 is identical to a mutation in KRT6, identified as the cause of pachyonychia congenita (148041.0001). Due to common codon usage, several type II keratins possess the ACC trinucleotide repeat and thus this may represent a mutational ‘hotspot’ in these genes.

Terrinoni et al. (2000) reported a heterozygous 3-bp insertion (ACA) in the K4 gene in affected members of an Italian family with white sponge nevus. The insertion occurred between basepairs 458 and 459 and resulted in the insertion of a glutamine residue between the second and third amino acids of the helix initiation motif of the 1A alpha-helical domain. The phenotype was considered mild, as only part of the buccal and labial mucosa was involved.

Steatocystoma multiplex (SM) is an uncommon autosomal dominant disorder involving the pilosebaceous unit (Authored by Mary Bane, MD, Chief, Assistant Professor, Department of Dermatology, Wright-Patterson USAF MedicalCenter). This condition (described by Jamieson in 1873) is characterized by the development of numerous sebum-containing dermal cysts. The cysts can be widespread and difficult to treat.

SM is a familial disorder of the pilosebaceous unit with an autosomal dominant transmission. The lesions are a nevoid formation of abortive hair follicles at the site where the sebaceous glands attach. No evidence exists of blockage of a follicular duct. Electron microscopic studies demonstrate cyst wall cells undergoing trichilemmal keratinization similar in manner to the isthmus portion of the outer hair sheath. Its relationship to the development of sebaceous glands and its presentation at puberty suggest a hormonal trigger for the lesion growth. This also corresponds with the more common distribution of cysts on the upper-central torso. SM frequently is associated with vellus hair cysts (VHC) in the same patient. These 2 cyst types have the same distribution and timing in presentation, and may represent points along a continuum in which some would include trichostasis. SM also has been seen in patients with pachyonychia congenita type 2 (PC-2) in which a keratin 17 mutation was localized. This mutation has also been reported in SM without features of PC-2. In SM associated with VHC, however, no mutation in K17 has been found. SM may be a heterogeneous group of genotypes that have similar phenotypic or clinical presentations.

The disclosed NOV2 nucleic acid of the invention encoding a Keratin 4-like protein includes the nucleic acid whose sequence is provided in Table 1A, C and E or a fragment thereof. The invention also includes a mutant or variant nucleic acid any of whose bases may be changed from the corresponding base shown in Table 1A, C, or E while still encoding a protein that maintains its Keratin 4-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV2 protein of the invention includes the Keratin 4-like protein whose sequence is provided in Table 1B or 1E. The invention also includes a mutant or variant protein any of whose residues may be changed from the corresponding residue shown in Table 1B or 1E while still encoding a protein that maintains its Keratin 4-like activities and physiological functions, or a functional fragment thereof. In the mutant or variant protein, up to about 60% percent of the residues may be so changed.

The invention further encompasses antibodies and antibody fragments, such as F_{ab} or (F_{ab})_{2,} that bind immunospecifically to any of the proteins of the invention.

The above defined information for this invention suggests that this Keratin 4-like protein (NOV2) may function as a member of a “Keratin 4 family”. Therefore, the NOV2 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV2 nucleic acids and proteins of the invention are useful in potential therapeutic applications implicated in cancer including but not limited to various pathologies and disorders as indicated below. For example, a cDNA encoding the Keratin 4-like protein (NOV2) may be useful in gene therapy, and the keratin 4-like protein (NOV2) may be useful when administered to a subject in need thereof. By way of nonlimiting example, the compositions of the present invention will have efficacy for treatment of patients suffering from Steatocystoma multiplex, Muscular dystrophy, Lesch-Nyhan syndrome, Myasthenia gravis and Breast Cancer other muscular disorders. The NOV2 nucleic acid encoding the keratin 4-like protein of the invention, or fragments thereof, may further be useful in diagnostic applications, wherein the presence or amount of the nucleic acid or the protein are to be assessed.

NOV2 nucleic acids and polypeptides are further useful in the generation of antibodies that bind immuno-specifically to the novel NOV2 substances for use in therapeutic or diagnostic methods. These antibodies may be generated according to methods known in the art, using prediction from hydrophobicity charts, as described in the “Anti-NOVX Antibodies” section below. The disclosed NOV2 protein has multiple hydrophilic regions, each of which can be used as an immunogen. In one embodiment, a contemplated NOV2 epitope is from about amino acids 70 to 110. In another embodiment, a NOV2 epitope is from about amino acids 430 to 460. In an additional embodiment, NOV2an epitope is from about amino acids 470 to 52. These novel proteins can be used in assay systems for functional analysis of various human disorders, which will help in understanding of pathology of the disease and development of new drug targets for various disorders.

NOV3

A disclosed NOV3 nucleic acid of 1113 nucleotides (also referred to as SC10341332_A) encoding a novel Collagen-like protein is shown in Table 3a. An open reading frame was identified beginning with a ATG initiation codon at nucleotides 29-31 and ending with a TGA codon at nucleotides 1076-1078. Putative untranslated regions upstream from the initiation codon and downstream from the stop codon are underlined in Table 3A The start and stop codons are in bold letters.

TABLE 3A
NOV3 Nucleotide Sequence

| | |
|---|---|
| GAAAATGAAAACATGAGTGAGTGCGGAGATGCCAGCTGCCTCTGCATCTTCTCCAGGCATTGAACACCTCACATGCTGCA | (SEQ ID NO:11) |
| AGGCAGGGTGGGGTCTTGTGTGCTGTCATATTTCACAGGGTGACGATGGGTCACATTTTCTCATCAGTCTCTGTTTCCTC | |
| TTGCTTTCCTCAGATGTGCTCTGTCCTAGCGTTCGTGTAGAAGGAGATCGCTTTAAGCACACCAATGGAGGAACCAAGGA | |
| AATCACAGGTTTGGACCTGATGGATTTGTTCAGTGTGAAGGAAATCTTGGGGAAGAGAGAGAATGGAGCTCAGAGTTCCT | |
| ATGTACGGATGGGATCCTTCCCTGTGGTGCAAAGTACTGAGGATGTGTTCCCCCAAGGTTTACCTGATGAGTACGCCTTT | |
| GTCACAACCTTCCGGTTCAGGAAAACCTCTCGGAAGGAAGACTGGTATATCTGGCAGGTCATCGACCAGTACGGCATCCC | |
| ACAGGTCTCCATCCGGCTGGATGGTGAAAACAAGGCAGTCGAGTACAACGCTGTGGGTGCCATGAAAGATGCTGTCAGGG | |
| TGGTCTTCCGAGGTTCTCGGGTCAATGACCTCTTTGACCGGGACTGGCACAAGATGGCCCTGAGCATCCAGGCCCAGAAC | |
| GTCTCCCTGCACATTGACTGTGCGCTGGTGCAGACACTACCCATCGAGGAACGGGAGAACATTGACATCCAGGGCAAGAC | |
| TGTGATTGGCAAGCGCCTCTACGACAGTGTGCCCATTGACGTGAGTACCAGGGGGCCTTCTGCAGCCCAGGTTCTCAGGC | |
| CACCGGGGAGAAGCTTGGGAGCTAAGTGCCCTCAGTGTTCTCCACACCTGCATGAACCAGGTACCAAATCATCACCTTGG | |
| ACCGTCTTAGAAGGAAAGACCCTGACTCAGAAAACTGCCATATTTGAACCCCAATTCACCATCACCCATGTATTAACTCA | |
| TTCGGTTATTCAACCATTCCATCAATCATTCATCACATATACATTGAGCACCTACTATGTGCCAGGCACTGTGCTCTGCA | |
| CTGGGGACACCGGTACCCGCAAAAGAGAGCAAGACTGACATGGATCTGGCCGCATAAACAATTTTAGAGAAAAG | |

The disclosed NOV3 nucleic acid sequence maps to chromosome 8 and has 95 of 136 bases (69%) identical to a EH domain containing 2 (EHD2) mRNA from Homo sapiens (GENBANK-ID: AF181263).

A disclosed NOV3 protein (SEQ ID NO: 12) encoded by SEQ ID NO: 11 has 349 amino acid residues, and is presented using the one-letter code in Table 3B. Signal P, Psort and/or Hydropathy results predict that NOV3 has a signal peptide, and is likely to be localized to the endoplasmic reticulum with a certainty of 0.6500. In other embodiments NOV3 is also likely to be localized to microbody (peroxisome) with a certainty of 0.2965, to the mitochondrial membrane space with a certainty of 0.1000, or to the plasma membrane with a certainty of 0.1000. The most likely cleavage point of the disclosed NOV3 polypeptide is after residue 59 of SEQ ID NO: 12.

TABLE 3B
Encoded NOV3 protein sequence.

| | |
|---|---|
| MPAASASSPGIEHLTCCKAGWGLVCCHISQGDDGSHFLISLCFLLLSSDVLCPSVRVEGDRFKHTNGGTKEITGLDLMDL | (SEQ ID NO:12) |
| FSVKEILGKRENGAQSSYVRMGSFPVVQSTEDVFPQGLPDEYAFVTTFRFRKTSRKEDWYIWQVIDQYGIPQVSIRLDGE | |
| NKAVEYNAVGAMKDAVRVVFRGSRVNDLFDRDWHKMALSIQAQNVSLHIDCALVQTLPIEERENIDIQGKTVIGKRLYDS | |
| VPIDVSTRGPSAAQVLRPPGRSLGAKCPQCSPHLHEPGTKSSPWTVLEGKTLTQKTAIFEPQFTITHVLTHSVIQPFHQS | |
| FITYTLSTYYVPGTVLCTGDTGTRKREQD | |

The disclosed NOV3 amino acid has 72 of 194 amino acid residues (37%) identical to, and 119 of 194 amino acid residues (61%) similar to, the 1142 amino acid residue A1(XIX) collagen CHAIN PRECURSOR protein from Homo sapiens (Q13676)

Exon linking data for NOV3 is found below in Example 1. TaqMan expression data for NOV3 is found below is Example 2.

NOV3 also has homology to the amino acid sequences shown in the BLASTP data listed in Table 3C.

TABLE 3C
BLAST results for NOV3

| Gene Index/ | | Length | Identity | Positives | |
|---|---|---|---|---|---|
| Identifier | Protein/Organism | (aa) | (%) | (%) | Expect |
| gi\|182387\| | Fibril-associated | 599 | 37 | 61 | e-36 |
| | collagen/human | | | | |
| gi\|624871\| | Collagen type | 1142 | 37 | 61 | e-36 |
| | XIX/human | | | | |
| gi\|2119157\| | Collagen type | 1142 | 37 | 61 | e-26 |
| | XIX/human | | | | |
| gi\|14756010 | Collagen type | 913 | 36 | 60 | e-35 |
| | XIX/human | | | | |
| gi\|10281667 | Collagen type | | 36 | 60 | e-35 |
| | XIX/human | | | | |

The homology of these sequences is shown graphically in the ClustalW analysis shown in Table 3D.

[image]

[image]

[image]

[image]

Tables 3E-F list the domain description from DOMAIN analysis results against NOV3. This indicates that the NOV3 sequence has properties similar to those of other proteins known to contain this domain.

TABLE 3E
Domain Analysis of NOV3

gnl | Pfam | pfam02210, TSPN, Thrombospondin N-terminal -like domain. (Thro)

| | | | | |
|---|---|---|---|---|
| NOV3 | 74 | GLDLMDLFSVKEILGK--RENGAQSSYV--RMGSFPVV-QSTEDVFPQGLPDEYAFVTTF | 128 | (SEQ ID NO: 12) |
| Thro | 1 | GQDLLQVFDLPESSFSVRKGVGLHGSSPAYRFGKPAVVSQPTRTLFPSGLPEDFSLLTTF | 60 | (SEQ ID NO: 33) |
| NOV3 | 129 | RFRKTSRKEDWYIWQVIDQYGIPQVSIRLDGENKAVEYNAVGAMKDAVRVVFRGSRVNDL | 188 | |
| Thro | 61 | RQAPKSR---GVLFAIYDAQNVRQLGLEVNGRANTLLLRYQGVDGKQNTVSFRNL---PL | 114 | |
| N0V3 | 189 | FDRDWHKMALSIQAQNVSLHIDCALVQTLPIEER-ENIDIQGKTVIGKRL--YDSVPIDV | 245 | |
| Thro | 115 | ADGQWHKLALSVSGESATLYVDCNEIDSRPLDRPFPPIDTDGIEVRGAQAADEKKFQGDL | 174 | |

TABLE 3F
Domain Analysis of NOV3

gnl | Pfam | pfam01119, DNA_mis_repair, DNA mismatch repair protein. Also
known as the mutL/hexB/PMS1 family. (DNAr)

| | | | | |
|---|---|---|---|---|
| NOV3: | 129 | RFRKTSRKEDWYIWQVIDQYGI--PQVSIRLDGENKAVEY--NAVGAMKDAVRVVFRGSR | 184 | (SEQ ID NO: 12) |
| DNRr: | 18 | KFLKSPKKEFRKILDLLQRYALIHPNVSFSLTKEGKALLQLKTSPSSLKERIRSVFGTAV | 77 | (SEQ ID NO: 34) |
| NOV3: | 185 | VNDLFDRDWHKMALSI 200 | | |
| DNRr: | 78 | LKNLIPFEEKDGDFRI 93 | | |

The collagens are the major structural glycoproteins of connective tissues. A unique primary structure and a multiplicity of post-translational modification reactions are required for normal fibrillogenesis. The post-translational modifications include hydroxylation of prolyl and lysyl residues, glycosylation, folding of the molecule into triple-helical conformation, proteolytic conversion of precursor procollagen to collagen, and oxidative deamination of certain lysyl and hydroxylysyl residues. Any defect in the normal mechanisms responsible for the synthesis and secretion of collagen molecules or the deposition of these molecules into extracellular fibers could result in abnormal fibrillogenesis; such defects could result in a connective tissue disease. Recently, defects in the regulation of the types of collagen synthesized and in the enzymes involved in the post-translational modifications have been found in heritable diseases of connective tissue. Thus far, the primary heritable disorders of collagen metabolism in man include lysyl hydroxylase deficiency in Ehlers-Danlos syndrome type VI, p-collagen peptidase deficency in Ehlers-Danlos syndrome type VII, decreased synthesis of type III collagen in Ehlers-Danlos syndrome type IV, lysyl oxidase deficency in S-linked cutis laxa and Ehlers-Danlos syndrome type V, and decreased synthesis of type I collagen in osteogenesis imperfecta.

Distinct collagen subtypes are recognized by specific cell surface receptors. Two of the best known collagen receptors are members of the integrin family and are named alpha1beta1 and alpha2beta1. Integrin alpha1beta1 is abundant on smooth muscle cells, whereas the alpha2beta1 integrin is the major collagen receptor on epithelial cells and platelets. Many cell types, such as fibroblasts, osteoblasts, chondrocytes, endothelial cells, and lymphocytes may concomitantly express both of the receptors. Alpha1beta1 and alpha2beta1 integrins have differences in their ligand binding specificity. Furthermore, the two receptors are connected to distinct signaling pathways and their ligation may lead to opposite cellular responses. PMID: 10963992

Connective tissues maintain shape against external and internal stress. They are molecular hierarchies in which fundamental building units come together in tiers of increasing complexity and mutual interactions, based on information carried in the precursor molecules secreted by cells. The collagen fibril is the end product of well-understood self-aggregation controlled by its amino acid sequences, but the interfibrillar amorphous ground substance has not hitherto been seen as structured by analogous aggregations prescribed by the primary structures of the characteristic glycosaminoglycans dissolved therein. Transmission electron microscopy with morphometry and stereology has demonstrated their existence in tissues. Nuclear magnetic resonance defined their secondary structures, rotary shadowing electron microscopy delineated their aggregates in vitro, and molecular dynamics stimulations showed how the latter can spring from the former. The driving forces to aggregation are hydrophobic and hydrogen bonding, offset by electrostatic repulsion between polyanionic charges. The relative stabilities of the aggregates are determined by this balance, and hence by the position and number of their charges, particularly the sulfate ester groups. Corneal stroma is a system of collagen fibrils, highly ordered to ensure transparency, in which glycosaminoglycan aggregates are suggested to determine the ordered spacing as yardsticks in a way that has parallels in all connective tissues.

Recent biochemical and immunohistochemical studies have described several components of basement membranes including heparan sulfate proteoglycan, 2 high molecular weight glycoproteins (fibronectin and laminin), and 2 collagen types (IV and V). These collagens have several properties which distinguish them from other types that are located in the interstitium: (a) type IV forms an amorphous, felt-like matrix, and neither IV nor V is found in large, cross-banded fibrils, (b) both have an increased content of hydrophobic amino acids, (c) the precursor (pro) forms are larger than those of interstitial collagens, (d) type IV contains interruptions within the triple helix, and e) both IV and V are resistant to human skin collagenase but are substrates for selected neutral proteases derived from mast cells, macrophages, and granulocytes. By immunofluorescence staining, type IV collagen has been localized to basement membranes at the dermal-epidermal junction, in capillaries, and beneath endothelial cells in larger vessels. Ultrastructurally it has been shown to be a specific component of the lamina densa. Type V collagen has been localized to the pericellular matrices of several cells types and may be specific for extramembranous structures which are closely associated with basal laminae. Other collagenous proteins have been described which may be associated with the extracellular matrix. One of these is secreted by endothelial cells in culture and by peptide mapping represents a novel collagen type. It is secreted under ascorbate-free conditions and is highly sensitive to proteolytic degradation. It has been proposed that a dynamic reciprocity exists between cells and their extracellular matrix which partially determines cell shape, biosynthesis, migration, and attachment. Examples of phenotypic modulation in several of these phenomena have been shown with endothelial cells grown on different substrates and isolated from different vascular environments.

The disclosed NOV3 nucleic acid of the invention encoding a Collagen-like protein includes the nucleic acid whose sequence is provided in Table 3A or a fragment thereof. The invention also includes a mutant or variant nucleic acid any of whose bases may be changed from the corresponding base shown in Table 3A while still encoding a protein that maintains its Collagen-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV3 protein of the invention includes the Collagen-like protein whose sequence is provided in Table 3B. The invention also includes a mutant or variant protein any of whose residues may be changed from the corresponding residue shown in Table 3B while still encoding a protein that maintains its Collagen-like activities and physiological functions, or a functional fragment thereof. In the mutant or variant protein, up to about 13% percent of the residues may be so changed.

The protein similarity information, expression pattern, and map location for the Collagen-like protein and nucleic acid (NOV3) disclosed herein suggest that NOV3 may have important structural and/or physiological functions characteristic of the collagen-like family. Therefore, the NOV3 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV3 nucleic acids and proteins of the invention are useful in potential diagnostic and therapeutic applications implicated in various diseases and disorders described below. For example, the compositions of the present invention will have efficacy for treatment of patients suffering from vascular disorders, hypertension, skin disorders, renal disorders including Alport syndrome, immunological disorders, inflammation including irritable bowel disease, and tissue injury, cancers, fibrosis disorders, bone diseases, Ehlers-Danlos syndrome type VI, VII, type IV, S-linked cutis laxa and Ehlers-Danlos syndrome type V, osteogenesis imperfecta, Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Ataxia-telangiectasia, Leukodystrophies, Behavioral disorders, Addiction, Anxiety, Pain, and Neuroprotection and/or other pathologies. The NOV3 nucleic acid, or fragments thereof, may further be useful in diagnostic applications, wherein the presence or amount of the nucleic acid or the protein are to be assessed.

NOV3 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV3 protein have multiple hydrophilic regions, each of which can be used as an immunogen. In one embodiment, contemplated NOV3 epitope is from about amino acids 1 to 30. In another embodiment, a NOV3 epitope is from about amino acids 30 to 60. In additional embodiments, NOV3 epitopes are from about amino acids 80 to 100, from about amino acids 210 to 230, and from about amino acids 300 to 340. This novel protein also has value in development of powerful assay system for functional analysis of various human disorders, which will help in understanding of pathology of the disease and development of new drug targets for various disorders.

NOV4

A disclosed NOV4 nucleic acid of 395 nucleotides (also referred to as GMAC018494_A) encoding a novel cystatin B-like protein is shown in Table 4a. An open reading frame was identified beginning with a ATG initiation codon at nucleotides 51-53 and ending with a TAA codon at nucleotides 324-326. Putative untranslated regions upstream from the initiation codon and downstream from the stop codon are underlined in Table 4A The start and stop codons are in bold letters.

TABLE 4A
NOV4 Nucleotide Sequence

| | |
|---|---|
| ACATCAACTCTCTGGTCCTTTCTGCCCTGTCCCCTCCCACTGCTACCACGATGACGTGTGGGGTGTCCCCT | (SEQ ID NO: 13) |
| GCCATGCTTGCCACTGAAGAGACCCAGGACGTTGCTAACCAGGTGAAGAGCCTCAATTATGAGAAAAAAAT | |
| CAAGAAGTTCCCTATTTTTAAGGCTGTGGTATTCAAGAGCCAGGTGGTCACAGGGACAAACTTCCACGTTG | |
| CTGATAACATCGTATACTTCCAAGTATTTAACAGTCTCCCTCATGAAAACAAGCCCTTGACCTCATCTGAC | |
| TACCAGCCCAAGGCCAACCAGGAGACAAGCTGCTGTATTTCTAAGTCCTGATTTTGAAGAAGGCCCTTCATTC | |
| ATATGACTAAGACTTAAAACAATTCTCTGTTTGAAGCCAC | |

The disclosed NOV4 nucleic acid sequence maps to chromosome 3 and has has 151 of 207 bases (72%) identical to a Cystatin B mRNA from Homo sapiens (GENBANK-ID: HUMCST4BA). The NOV4 nucleic acid disclosed in this invention is expressed in at least the following tissues: lung, brain, kidney, and skin.

A disclosed NOV4 protein (SEQ ID NO: 14) encoded by SEQ ID NO: 13 has 91 amino acid residues, and is presented using the one-letter code in Table 4B. Signal P, Psort and/or Hydropathy results predict that NOV4 does not have a signal peptide, and is likely to be localized to the cytoplasm with a certainty of 0.4500. In other embodiments NOV4 is also likely to be localized to microbody (peroxisome) with a certainty of 0.1501, to the mitochondrial matrix space with a certainty of 0.1000, or to the lysosome (lumen) with a certainty of 0.1000.

TABLE 4B
Encoded NOV4 protein sequence.

| | |
|---|---|
| MTCGVSPAMLATEETQDVANQVKSLNYEKKIKKFPIFKAVVFKSQVVTGTNFHVADNIVYFQVFN | (SEQ ID NO: 14) |
| SLPHENKPLTSSDYQPKANQDKLLYF | |

The disclosed NOV4 amino acid has 57 of 98 amino acid residues (58%) identical to, and 68 of 98 amino acid residues (69%) similar to, the 98 amino acid residue Cystatin B protein from Homo sapiens (P04080).

Exon linking data for NOV4 is found below in Example 1. TaqMan expression data for NOV4 is found below is Example 2. SNP data for NOV4 is found below in Example 3.

NOV4 also has homology to the amino acid sequences shown in the BLASTP data listed in Table 4C.

TABLE 4C
BLAST results for NOV4

| Gene Index/ | | Length | Identity | Positives | |
|---|---|---|---|---|---|
| Identifier | Protein/Organism | (aa) | (%) | (%) | Expect |
| gi\|209383\| | MS-2 polstefin B | 100 | 58 | 68 | e-17 |
| | protein/synthetic | | | | |
| gi\|4503117\| | Cystatin B/human | 98 | 58 | 69 | e-17 |
| gi\|4520365\| | Cystatin B/ | 98 | 53 | 67 | e-17 |
| | oryctolagus c. | | | | |
| gi\|68783\| | Cystain b/human | 98 | 58 | 68 | e-17 |
| gi\|494619\| | Papain chain I | 98 | 56 | 67 | e-16 |

The homology of these sequences is shown graphically in the ClustalW analysis shown in Table 4D.

NOV4 (SEQ ID NO: 14)

gi|2093831 |(SEQ ID NO: 96)

gi|4503117|(SEQ ID NO: 97)

gi|45203651 |(SEQ ID NO: 98)

gi|68783|(SEQ ID NO: 99)

gi|4946191 |(SEQ ID NO: 100)

[image]

Table 4E lists the domain description from DOMAIN analysis results against NOV4. This indicates that the NOV4 sequence has properties similar to those of other proteins known to contain this domain.

cystatin (PFAM accession No: PF00031) (CYST)

| CYST | | GglspaddNendpevqeaadfAvae.yNeksdgykfelvevvraksQ | | (SEQ ID NO: 35) |
|---|---|---|---|---|
| | | \|+\|\|\| +++\|+\|++\|+ +++ +\|++\| +\|\| ++++\| \|\|\| | | |
| NOV4 | 3 | CGVSPAML--ATEETQDVANQVKSLnYEKKI--KKFPIFKAVVFKSQ | 45 | (SEQ ID NO: 14) |
| CYST | | VvaGtltnY | | |
| | | \|\| \|\| \|+ | | |
| NOV4 | 46 | VVTGT--NF 52 | | |

The cystatin “superfamily” encompasses proteins that contain multiple cystatin-like sequences. Some of the members are active cysteine protease inhibitors, while others have lost or perhaps never acquired this inhibitory activity. In recent years, several new members of the superfamily have characterized, including proteins from insects and plants. Based on partial amino acid homology, new members, such as the invariant chain (Ii), and the transforming growth factor-beta receptor type II (TGF-beta receptor II) may, in fact, represent members of an emerging family within the superfamily that may have used some common building blocks to form functionally diverse proteins. Cystatin super-family members have been found throughout evolution and members of each family of the superfamily are present in mammals today. In this review, the new and older, established members of the family are arranged into a possible evolutionary order, based on sequence homology and functional similarities.

Immunohistochemica and quantitative immunochemical methods were used to demonstrate the presence of two cysteine proteinase inhibitors, cystatins A and B, in normal and diseased tissues. Cystatin A is expressed in squamous epithelia, neutrophil granulocytes, and dendritic reticulum cells of the lymphatic tissues. Its concentration is increased in inflammatory skin diseases and decreases after the malignization of squamous epithelia. Cystatin B is seen in wet squamous epithelia, and in the cells of monocyte-macrophage series, where its concentration varies depending on the activation state of the cells. In the malignant keratinocytes cystatin B follows the behaviour of cystatin A.

The cystatins inhibit most cysteine endopeptidases of the papain type, and also the exopeptidase dipeptidyl peptidase I. Each cystatin molecule has a single reactive site for all the peptidases it inhibits, but there are large differences in K(i) values for different combinations of cystatin and enzyme, and calpains are inhibited only by one of the segments of the kininogens. The cystatins have many important characteristics in common, but their differences in molecular structure imply different routes of biosynthesis, are associated with different in vivo distributions, and suggest a variety of physiological functions.

Treatment strategies based on the molecular biology of the epilepsies may soon become a reality. Critical steps in this process are identifying molecular genetic defects in specific epilepsies, understanding of the neurobiologic consequences of those defects, and developing methods to correct the molecular defects or their downstream consequences. Identification of molecular defects is easier in single-gene epilepsies than in those with complex inheritance, although the latter are more common. A number of epilepsies have been mapped and, in two cases, specific genes have been identified. Unverricht-Lundborg disease is caused by defects in the cystatin B gene, with absence of the gene product. Autosomal dominant nocturnal frontal lobe epilepsy in some families is caused by mutations in the alpha4-subunit of the nicotinic acetylcholine receptor gene. In vitro studies suggest that the mutations lead to impaired function of the acetylcholine receptor, raising the possibility of cholinergic therapy for this condition. Advances in the molecular biology of the epilepsies are likely to change our understanding radically and to allow opportunities to develop innovative new treatments for epilepsy.

The cornified cell envelope is a highly insoluble and extremely tough structure formed beneath the cell membrane during terminal differentiation of keratinocytes. Its main function is to provide human skin with a protective barrier against the environment. Sequential cross-linking of several integral components catalyzed by transglutaminases leads to a gradual increase in the thickness of the envelope and underscores its rigidity. Key structural players in this cross-linking process include involucrin, loricrin, SPRRs, elafin, cystatin A, S100 family proteins, and some desmosomal proteins. The recent identification of genetic skin diseases with mutations in the genes encoding some of these proteins, including transglutaminase 1 and loricrin, has disclosed that abnormal cornified cell envelope synthesis is significantly involved in the pathophysiology of certain inherited keratodermas and reflects perturbations in the complex, yet highly orderly process of cornified cell envelope formation in normal skin biology.

Cerebral amyloid angiopathy (CAA) is a significant risk factor for hemorrhagic stroke in the elderly, and occurs as a sporadic disorder, as a frequent component of Alzheimer's disease, and in several rare, hereditary conditions. The most common type of amyloid found in the vasculature of the brain is beta-amyloid (A beta), the same peptide that occurs in senile plaques. A paucity of animal models has hindered the experimental analysis of CAA. Several transgenic mouse models of cerebral beta-amyloidosis have now been reported, but only one appears to develop significant cerebrovascular amyloid. However, well-characterized models of naturally occurring CAA, particularly aged dogs and non-human primates, have contributed unique insights into the biology of vascular amyloid in recent years. Some non-human primate species have a predilection for developing CAA; the squirrel monkey (Saimiri sciureus), for example, is particularly likely to manifest beta-amyloid deposition in the cerebral blood vessels with age, whereas the rhesus monkey (Macaca mulatta) develops more abundant parenchymal amyloid. These animals have been used to test in vivo beta-amyloid labeling strategies with monoclonal antibodies and radiolabeled A beta. Species-differences in the predominant site of A beta deposition also can be exploited to evaluate factors that direct amyloid selectively to a particular tissue compartment of the brain. For example, the cysteine protease inhibitor, cystatin C, in squirrel monkeys has an amino acid substitution that is similar to the mutant substitution found in some humans with a hereditary form of cystatin C amyloid angiopathy, possibly explaining the predisposition of squirrel monkeys to CAA. The existing animal models have shown considerable utility in deciphering the pathobiology of CAA, and in testing strategies that could be used to diagnose and treat this disorder in humans.

The disclosed NOV4 nucleic acid of the invention encoding a Cystatin B-like protein includes the nucleic acid whose sequence is provided in Table 4A or a fragment thereof. The invention also includes a mutant or variant nucleic acid any of whose bases may be changed from the corresponding base shown in Table 4A while still encoding a protein that maintains its Cystatin B-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV4 protein of the invention includes the Cystatin B-like protein whose sequence is provided in Table 4B. The invention also includes a mutant or variant protein any of whose residues may be changed from the corresponding residue shown in Table 4B while still encoding a protein that maintains its Cystatin B-like activities and physiological functions, or a functional fragment thereof. In the mutant or variant protein, up to about 13% percent of the residues may be so changed.

The protein similarity information, expression pattern, and map location for the Cystatin B-like protein and nucleic acid (NOV4) disclosed herein suggest that NOV4 may have important structural and/or physiological functions characteristic of the cystatin B-like family. Therefore, the NOV4 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV4 nucleic acids and proteins of the invention are useful in potential diagnostic and therapeutic applications implicated in various diseases and disorders described below. For example, the compositions of the present invention will have efficacy for treatment of patients suffering from Alzheimer's disease (AD). Epilepsies, Unverricht-Lundborg disease; skin disorders, differentiation of keratinocytes; Cerebral amyloid angiopathy (CAA), amyloidosis, and hemorrhagic stroke; inflammatory disorders, allergic inflammation; cancer; HIV and AIDS; kidney diseases; Neurological disorders and/or other pathologies. The NOV4 nucleic acid, or fragments thereof, may further be useful in diagnostic applications, wherein the presence or amount of the nucleic acid or the protein are to be assessed.

NOV4 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV4 protein have multiple hydrophilic regions, each of which can be used as an immunogen. In one embodiment, contemplated NOV4 epitope is from about amino acids 10 to 15. In another embodiment, a NOV4 epitope is from about amino acids 38 to 65. This novel protein also has value in development of powerful assay system for functional analysis of various human disorders, which will help in understanding of pathology of the disease and development of new drug targets for various disorders.

NOV5

A disclosed NOV5 nucleic acid of 1152 nucleotides (also referred to as GMAC009404_A) encoding a novel serotonin receptor-like protein is shown in Table 5A. An open reading frame was identified beginning with a ATG initiation codon at nucleotides 5-7 and ending with a TGA codon at nucleotides 1142-1144. Putative untranslated regions upstream from the initiation codon and downstream from the stop codon are underlined in Table 5A. The start and stop codons are in bold letters.

TABLE 5A
NOV5 Nucleotide Sequence

| | |
|---|---|
| CGCCATGGAGGCCGCTAGCCTTTCAGTGGCCACCGCCGGCGTTGCCCTTGCCCTGGGACCCGACCAGC | (SEQ ID NO: 15) |
| AGCGGACCCGGGACCCCAAGCCCGAGAGGGATACTCGGTTCGACCCCGAGCGGCGCCGTCCTGCCGGGCC | |
| GAGGGCCGCCCTTCTCTGTCTTCACGGTCCTGGTGGTGACGCTGCTAGTGCTGCTGATCGCTGCCACTTT | |
| CCTGTGGAACCTGCTGGTTCCGGTCACCATCCCGCGGGTCCGTGCCTTCCACCGCGTGCCGCATAACTTG | |
| GTGGCCTCGACGGCCGTCTCGGACGAACTAGTGGCAGCGCTGGCGATGCCACCGAGCCTGGCGAGTGAGC | |
| TGTCGACCGGGCGACGTCGGCTGCTGGGCCGGAGCCTGTGCCACGTGTGGATCTCCTTCGACGCCGGAGC | |
| CTGTCTGTGCTGCCCCGCCGGCCTCGGGAACGTGGCGGCCATCGCCCTGGGCCGCGACGGGGCCATCACA | |
| CGGCACCTGCAGCACACGCTGCGCACCCGCAGCCGCGCCTCGTTGCTCATGATCGCGCTCGCCCGGGTGC | |
| CGTCGGCGCTCATCGCCCTCGCGCCGCTGCTCTTTGGCCGGGGCGAGGTGTGCGACGCTCGGCTCCAGCG | |
| CTGCCAGGTGAGCCGGGAACCCTCCTATGCCGCCTTCTCCACCCGCGGCGCCTTCCACCTGCCGCTTGGC | |
| GTGGTGCCGTTTGTCTACCGGAAGATCTACGAGGCGGCCAAGTTTCGTTTCGGCCGCCGCCGGAGAGCTG | |
| TGCTGCCGTTGCCGGCCACCATGCAGGTGAGGTCCAACGTAAAGGAAGCACCTGATGAGGCTGAAGTGGT | |
| GTTCACGGCACATTGCAAAGCAACGGTGTCCTTCCAGGTGAGCGGGGACTCCTGGCGGGAGCAGAAGGAG | |
| AGGCGAGCAGCCATGATGGTGGGAATTCTGATTGGCGTGTTTGTGCTGTGCTGGATCCCCTTCTTCCTGA | |
| CGGAACTCATCAGCCCACTCTGTGCCTGCAGCCTGCCCCCCATCTGGAAAAGCATATTTCTGTGGCTTGG | |
| CTACTCCAATTCTTTCTTCAACCCCCTGATTTACACAGCTTTTAACAAGAACTACAACAATGCCTTCAAG | |
| AGCCTCTTTACTAAGCAGAGATGAACACAGGG | |

The disclosed NOV5 nucleic acid sequence maps to chromosome 2 and has 304 of 346 bases (87%) identical to a Serotonin Receptor-like 1 mRNA from humans (GENBANK-ID: X69867). The NOV5 nucleic acid disclosed in this invention is expressed in at least the following tissues: most peripheral organs and brain.

A disclosed NOV5 protein (SEQ ID NO: 16) encoded by SEQ ID NO: 15 has 379 amino acid residues, and is presented using the one-letter code in Table 5B. Signal P, Psort and/or Hydropathy results predict that NOV5 has a signal peptide, and is likely to be localized to the plasma membrane with a certainty of 0.6400. In other embodiments NOV5 is also likely to be localized to the golgi body with a certainty of 0.4600, or to the endoplasmic reticulum (membrane) with a certainty of 0.3700. The most likely cleavage site is after residue 23 or SEQ ID NO: 16.

TABLE 5B
Encoded NOV5 protein sequence.

| | |
|---|---|
| MEAASLSVATAGVALALGPETSSGPGTPSPRGILGSTPSGAVLPGRGPPFSVFTVLVVTLLVLLIAATFL | (SEQ ID NO:16) |
| WNLLVPVTIPRVRAFHRVPHNLVASTAVSDELVAALAMPPSLASELSTGRRRLLGRSLCHVWISFDAGAC | |
| LCCPAGLGNVAAIALGRDGAITRHLQHTLRTRSRASLLMIALARVPSALIALAPLLFGRGEVCDARLQRC | |
| QVSREPSYAAFSTRGAFHLPLGVVPFVYRKIYEAAKFRFGRRRRAVLPLPATMQVRSKVKEAPDEAEVVF | |
| TAHCKATVSFQVSGDSWREQKERRAAMMVGILIGVFVLCWIPFFLTELISPLCACSLPPIWKSIFLWLGY | |
| SNSFFNPLIYTAFNKNYNNAFKSLFTKQR | |

The disclosed NOV5 amino acid has 296 of 379 amino acid residues (78%) identical to, and 316 of 379 amino acid residues (83%) similar to, the 370 amino acid residue Serotonin Receptor-like 3 protein from Rattus norvegicus (ACC: P35365).

Exon linking data for NOV5 is found below in Example 1. TaqMan expression data for NOV5 is found below is Example 2.

NOV5 also has homology to the amino acid sequences shown in the BLASTP data listed in Table 5C.

TABLE 5C
BLAST results for NOV5

| Gene Index/ | | Length | Identity | Positives | |
|---|---|---|---|---|---|
| Identifier | Protein/Organism | (aa) | (%) | (%) | Expect |
| gi\|543730\| | Serotonin | 370 | 78 | 83 | e-142 |
| | receptor/rat | | | | |
| gi\|6754260\| | Serotonin | 370 | 78 | 83 | e-142 |
| | receptor 5B/ | | | | |
| | mouse | | | | |
| gi\|543453 | Serotonin | 369 | 78 | 83 | e-141 |
| | receptor 5B/rat | | | | |
| gi\|13236497 | Serotonin | 357 | 62 | 72 | e-108 |
| | receptor 5a/ | | | | |
| | human | | | | |
| gi\|6981062\| | Serotonin | 357 | 63 | 73 | e-102 |
| | receptor REC17/ | | | | |
| | rat | | | | |

The homology of these sequences is shown graphically in the ClustalW analysis shown in Table 5D.

NOV5 (SEQ ID NO: 16)

gi|5437301 |(SEQ ID NO: 101)

gi|67542601 |(SEQ ID NO: 102)

gi|5434531 |(SEQ ID NO: 103)

gi|32364971 |(SEQ ID NO: 104)

gi|6981062 |(SEQ ID NO: 105)

[image]

[image]

Table 5E list domain description from DOMAIN analysis results against NOV5. This indicates that the NOV5 sequence has properties similar to those of other proteins known to contain this domain.

TABLE 5E
NOV5 DOMAIN ANALYSIS

gnl | Pfam | pfam00001, 7tm_1, 7 transmembrane receptor (rhodopsin family) (7-TMR).

| | | | | |
|---|---|---|---|---|
| NOV5: | 72 | NLLVPVTIPRVRAFHRVPHNLVASTAVSDELVAALAMPPSLASELSTGRRRLLGRSLCHV | 131 | (SEQ ID NO: 16) |
| 7-TMR: | 2 | NLLVILVILRTKKLRTPTNIFLLNLAVADLLFLLTLPPWALYYLVGGDWVFGDAL--CKL | 59 | (SEQ ID NO: 36) |
| NOV5: | 132 | WISFDAGACLCCPAGLGNVAAIALGRDGAITRHLQHTLRTRSRASLLMIALARVPSALIA | 191 | |
| 7-TMR: | 60 | VGALF---VVNGYASILLLTAISIDRYLAIVHPLRYRRIRTPRRAKVLILLVWVLALLLS | 116 | |
| NOV5: | 192 | LAPLLFG------RGEVCDARLQRCQVSPEPSYAAFSTRGAFHLPLGVVPFVYRKIYEAA | 245 | |
| 7-TMR: | 117 | LPPLLFSWLRTVEEGNTTVCLIDFPEESVKRSYVLLSTLVGFVLPLLVILVCYTRILRTL | 176 | |
| NOV5: | 246 | KFRFGRRRRAVLPLPATMQVRSKVKEAPDEAEVVFTAHCKATVSFQVSGDSWREQKERRA | 305 | |
| 7-TMR: | 177 | R----------------------------------------KRARSQRSLKRRSSSERKA | 196 | |
| NOV5: | 306 | AMNVGILIGVFVLCWIPFFLTELISPLCACS---LPPIWKSIFLWLGYSNSFFNPLIY | 360 | |
| 7-TMR: | 197 | AKMLLVVVVVFVLCWLPYHIVLLLDSLCLLSIWRVLPTALLITLWLAYVNSCLNPIIY | 254 | |

The neurotransmitter serotonin (5-hydroxytryptamine; 5-HT) exerts a wide variety of physiologic functions through a multiplicity of receptors and may be involved in human neuropsychiatric disorders such as anxiety, depression, or migraine. These receptors consist of 4 main groups, 5-HT-1, 5-HT-2, 5-HT-3, and 5-HT4, subdivided into several distinct subtypes on the basis of their pharmacologic characteristics, coupling to intracellular second messengers, and distribution within the nervous system.

Serotonin, acting through many receptors, can modulate the activity of neural reward pathways and thus the effects of various drugs of abuse. Rocha et al. (1998) examined the effects of cocaine in mice lacking one of the serotonin receptor subtypes, the 5-HT1B-receptor. They showed that mice lacking this receptor displayed increased locomotor responses to cocaine and were more motivated to self-administer cocaine. Rocha et al. (1998) proposed that even drug-naive 5-HT1B knockout mice are in a behavioral and biochemical state that resembles that of wildtype mice sensitized to cocaine by repeated exposure to the drug. This altered state may be responsible for their increased vulnerability to cocaine.

Serotonin receptors belong to the 7 transmembrane rhodopsin family. The rhodopsin-like GPCRs themselves represent a widespread protein family that includes hormone, neurotransmitter and light receptors, all of which transduce extracellular signals through interaction with guanine nucleotide-binding (G) proteins. Although their activating ligands vary widely in structure and character, the amino acid sequences of the receptors are very similar and are believed to adopt a common structural framework comprising 7 transmembrane (TM) helices. G-protein-coupled receptors (GPCRs) constitute a vast protein family that encompasses a wide range of functions (including various autocrine, paracrine and endocrine processes). They show considerable diversity at the sequence level, on the basis of which they can be separated into distinct groups. We use the term clan to describe the GPCRs, as they embrace a group of families for which there are indications of evolutionary relationship, but between which there is no statistically significant similarity in sequence. The currently known clan members include the rhodopsin-like GPCRs, the secretin-like GPCRs, the cAMP receptors, the fungal mating pheromone receptors, and the metabotropic glutamate receptor family.

The disclosed NOV5 nucleic acid of the invention encoding a Serotonin receptor-like protein includes the nucleic acid whose sequence is provided in Table 5A or a fragment thereof. The invention also includes a mutant or variant nucleic acid any of whose bases may be changed from the corresponding base shown in Table 5A while still encoding a protein that maintains its Serotonin receptor-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV5 protein of the invention includes the Serotonin receptor-like protein whose sequence is provided in Table 5B. The invention also includes a mutant or variant protein any of whose residues may be changed from the corresponding residue shown in Table 4B while still encoding a protein that maintains its Serotonin receptor-like activities and physiological functions, or a functional fragment thereof. In the mutant or variant protein, up to about 13% percent of the residues may be so changed.

The protein similarity information, expression pattern, and map location for the Serotonin receptor-like protein and nucleic acid (NOV5) disclosed herein suggest that NOV5 may have important structural and/or physiological functions characteristic of the serotonin receptor-like family. Therefore, the NOV5 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV5 nucleic acids and proteins of the invention are useful in potential diagnostic and therapeutic applications implicated in various diseases and disorders described below. For example, the compositions of the present invention will have efficacy for treatment of patients suffering from migraine, Alzheimer disease, eating disorder, anxiety-related disorder, epilepsy, retinoblastoma, schizophrenia, Tourette syndrome, autistic disorder, heart disorders, and/or other pathologies. The NOV5 nucleic acid, or fragments thereof, may further be useful in diagnostic applications, wherein the presence or amount of the nucleic acid or the protein are to be assessed.

NOV5 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV5 protein have multiple hydrophilic regions, each of which can be used as an immunogen. In one embodiment, contemplated NOV5 epitope is from about amino acids 1 to 15. In another embodiment, a NOV5 epitope is from about amino acids 45 to 80. In additional embodiments, NOV5 epitopes are from about amino acids 90 to 110, from about amino acids 120 to 150, from about amino acids 170 to 200, from about amino acids 220 to 240, and from about amino acids 300 to 350. This novel protein also has value in development of powerful assay system for functional analysis of various human disorders, which will help in understanding of pathology of the disease and development of new drug targets for various disorders.

NOV6

NOV6 includes two novel cold inducible glycoprotein 30-like proteins disclosed below. The disclosed sequences have been named NOV6a, and NOV6b. NOV6 is localized to human chromosome 10.

NOV6a

A disclosed NOV6a nucleic acid of 815 nucleotides (also referred to as SC126404196_A) encoding a novel Cold inducible glycoprotein 30-like protein is shown in Table 6A. An open reading frame was identified beginning with an ATG initiation codon at nucleotides 1-3 and ending with a TGA codon at nucleotides 811-813. A putative untranslated region downstream from the stop codon is underlined in Table 6A. The start and stop codons are in bold letters.

TABLE 6A
NOV6a nucleotide sequence.

| | |
|---|---|
| ATGGTCACAGCCATGAATGTCTCACATGAAGTAAATCAGCTGTTCCAGCCCTATAACTTCGAGCTGTCCA | (SEQ ID NO:17) |
| AGGACATGAGGCCCTTTTTCGAGGAGTATTGGGCAACCTCATTCCCCATAGCCCTGATCTACCTGGTTCT | |
| CATCGCTGTGGGGCAGAACTACATGAAGGAACGCAAGGGCTTCAACCTGCAAGGGCCTCTCATCCTCTGG | |
| TCCTTCTGCCTTGCAATCTTCAGTATCCTGGGGGCAGTGAGGATGTGGGGCATTATGGGGACTGTGCTAC | |
| TTACCGGGGGCCTAAAGCAAACCGTGTGCTTCATCAACTTCATCGATAATTCCACAGTCAAATTCTGGTC | |
| CTGGGTCTTTCTTCTCAGCAAGGTCATAGAACTCGGTGACACAGCCTTCATCATCCTGCGTAAGCGGCCA | |
| CTCATCTTTATTCACTGGTACCACCACAGCACAGTGCTCGTGTACACAAGCTTTGGATACAAGAACAAAG | |
| TGCCTGCAGGAGGCTGGTTCGTCACCATGAACTTTGGTGTTCATGCCATCATGTACACCTACTACACTCT | |
| GAAGGCTGCCAACGTGAAGCCCCCCAAGATGCTGCCCATGCTCATCACCAGCCTGCAGATCTTGCAGATG | |
| TTTGTAGGAGCCATCGTCAGCATCCTCACGTACATCTGGAGGCAGGATCAGGGATGCCACACCACGATGG | |
| AACACTTATTCTGGTCCTTCATCTTGTATATGACCTATTTCATCCTCTTTGCCCACTTCTTCTGCCAGAC | |
| CTACATCAGGCCCAAGGTCAAAGCCAAGACCAAGAGCCAGTGAAG | |

In a search of sequence databases, it was found, for example, that a NOV6a nucleic acid sequence has has 654 of 816 bases (80%) identical to a Cold Inducible Glycoprotein 30-like 1 mRNA from Mus musculus (GENBANK-ID: U97107). Public nucleotide databases include all GenBank databases and the GeneSeq patent database.

The disclosed NOV6a polypeptide (SEQ ID NO: 18) encoded by SEQ ID NO: 17 has 270 amino acid residues and is presented in Table 6B using the one-letter amino acid code. Signal P, Psort and/or Hydropathy results predict that NOV6a has a signal peptide and is likely to be localized in the plasma membrane with a certainty of 0.6000. In other embodiments, NOV26 may also be localized to the Golgi body with a certainty of 0.4000, the endoplasmic reticulum membrane with a certainty of 0.3000, or in the mitochondrial inner membrane with a certainty of 0.0300.

Exon linking data for NOV6 can be found below in Example 1. Taqman data for NOV6 can be found below in Example 2.

TABLE 6B
Encoded NOV6a protein sequence.

| | |
|---|---|
| MVTAMNVSHEVNQLFQPYNFELSKDMRPFFEEYWATSFPIALIYLVLIAVGQNYMKERKGFNQLGPLILW | (SEQ ID NO:18) |
| SFCLAIFSILGAVRMWGIMGTVLLTGGLKQTVCFINFIDNSTVKFWSWVFLLSKVIELGDTAFIILRKRP | |
| LIFIHWYHHSTVLVYTSFGYKNKVPAGGWFVTMNFGVHAIMYTYYTLKAANVKPPKMLPMLITSLQILQM | |
| FVGAIVSILTYIWRQDQGCHTTMEHLFWSFILYMTYFILFAHFFCQTYIRPKVKAKTKSQ | |

The full amino acid sequence of the disclosed NOV6a protein was found have 187 of 271 amino acid residues (69%) identical to, and 225 of 271 amino acid residues (83%) similar to, the 271 amino acid residue Cold Inducible Glycoprotein 30-like 3 protein from Mus musculus (ACC: 035949). Public amino acid databases include the GenBank databases, SwissProt, PDB and PIR.

NOV6b

A disclosed NOV6b nucleic acid of 815 nucleotides (also referred to as SC126404196_A_da1; CG55866-01) encoding a novel Cold inducible glycoprotein 30-like protein is shown in Table 6C. An open reading frame was identified beginning with an ATG initiation codon at nucleotides 1-3 and ending with a TGA codon at nucleotides 811-813. A putative untranslated region downstream from the stop codon is underlined in Table 6C. The start and stop codons are in bold letters.

TABLE 6C
NOV6b nucleotide sequence.

| | |
|---|---|
| ATGGTCACAGCCATGAATGTCTCACATGAAGTAAATCAGCTGTTCCAGCCCTATAACTTCGAGCTGTCCA | (SEQ ID NO:19) |
| AGGACATGAGGCCCTTTTTCGAGGAGTATTGGGCAACCTCATTCCCCATAGCCCTGATCTACCTGGTTCT | |
| CATCGCTGTGGGGCAGAACTACATGAAGGAACGCAAGGGCTTCAACCTGCAAGGGCCTCTCATCCTCTGG | |
| TCCTTCTGCCTTGCAATCTTCAGTATCCTGGGGGCAGTGAGGATGTGGGGCATTATGGGGACTGTGCTAC | |
| TTACCGGGGGCCTAAAGCAAACCGTGTGCTTCATCAACTTCATCGATAATTCCACAGTCAAATTCTGGTC | |
| CTGGGTCTTTCTTCTCAGCAAGGTCATAGAACTCGGTGACACAGCCTTCATCATCCTGCGTAAGCGGCCA | |
| CTCATCTTTATTCACTGGTACCACCACAGCACAGTGCTCGTGTACACAAGCTTTGGATACAAGAACAAAG | |
| TGCCTGCAGGAGGCTGGTTCGTCACCATGAACTTTGGTGTTCATGCCATCATGTACACCTACTACACTCT | |
| GAAGGCTGCCAACGTGAAGCCCCCCAAGATGCTGCCCATGCTCATCACCAGCCTGCAGATCTTGCAGATG | |
| TTTGTAGGAGCCATCGTCAGCATCCTCACGTACATCTGGAGGCAGGATCAGGGATGCCACACCACGATGG | |
| AACACTTATTCTGGTCCTTCATCTTGTATATGACCTATTTCATCCTCTTTGCCCACTTCTTCTGCCAGAC | |
| CTACATCAGGCCCAAGGTCAAAGCCAAGACCAAGAGCCAGTGAAG | |

In a search of sequence databases, it was found, for example, that a NOV6b nucleic acid sequence has 653 of 816 bases (80%) identical to a gb:GENBANK-ID:MMU97107|acc:U97107.1 mRNA from Mus musculus (Mus musculus membrane glycoprotein CIG30 (Cig30) mRNA, complete cds).

The disclosed NOV6b polypeptide (SEQ ID NO: 20) encoded by SEQ ID NO: 19 has 270 amino acid residues and is presented in Table 6B using the one-letter amino acid code. Signal P, Psort and/or Hydropathy results predict that NOV6a has a signal peptide and is likely to be localized in the plasma membrane with a certainty of 0.6000. In other embodiments, NOV26 may also be localized to the Golgi body with a certainty of 0.4000, the endoplasmic reticulum membrane with a certainty of 0.3000, or in the mitochondrial inner membrane with a certainty of 0.0300.

TABLE 6D
Encoded NOV6b protein sequence.

| | |
|---|---|
| MVTAMNVSHEVNQLFQPYNFELSKDMRPFFEEYWATSFPIALIYLVLIAVGQNYMKERKGFNLQGPLILW | (SEQ ID NO:20) |
| SFCLAIFSILGAVRMWGIMGTVLLTGGLKQTVCFINFIDNSTVKFWSWVFLLSKVIELGDTAFIILRKRP | |
| LIFIHWYHHSTVLVYTSFGYKNKVPAGGWFVTMNPGVHAIMYTYYTLKAANVKPPKMLPMLITSLQILQM | |
| FVGAIVSILTYIWRQDQGCHTTMEHLFWSFILYMTYFILFAHFFCQTYIRPKVKAKTKSQ | |

The full amino acid sequence of the disclosed NOV6b protein was found to have 187 of 271 amino acid residues (69%) identical to, and 224 of 271 amino acid residues (82%) similar to, the 271 amino acid residue ptnr:SPTREMBL-ACC:O35949 protein from Mus musculus (Mouse) (COLD INDUCIBLE GLYCOPROTEIN 30 (MEMBRANE GLYCOPROTEIN CIG30)). Public amino acid databases include the GenBank databases, SwissProt, PDB and PIR.

The proteins encoded by the NOV6a, and 6b are very closely homologous as is shown in the alignment in Table 6F.

Homologies to any of the above NOV6 proteins will be shared by the other NOV protein insofar as they are homologous to each other as shown above. Any reference to NOV6 is assumed to refer to both the NOV6 proteins in general, unless otherwise noted.

The disclosed NOV6 polypeptide has homology to the amino acid sequences shown in the BLASTP data listed in Table 6E.

TABLE 6E
BLAST results for NOV6

| Gene Index/ | Protein/ | Length | Identity | Positives | |
|---|---|---|---|---|---|
| Identifier | Organism | (aa) | (%) | (%) | Expect |
| Gi\|10444345\| | CIG 30/human | 236 | 100 | 100 | e-118 |
| Gi\|6671752\| | CIG 30/mouse | 271 | 69 | 83 | e-91 |
| Gi\|12836437\| | Putative ORF/ | 360 | 71 | 84 | e-77 |
| | mouse | | | | |
| Gi\|13129088\| | Hypothetical | 265 | 45 | 64 | e-55 |
| | protein | | | | |
| | MGC5487 | | | | |
| Gi\|15799257\| | Fatty acyl | 267 | 45 | 65 | e-53 |
| | elongase/mouse | | | | |

The homology between these and other sequences is shown graphically in the ClustalW analysis shown in Table 6F.

[image]

[image]

DOMAIN results for NOV6 as disclosed in Table 6I, were collected from the Conserved Domain Database (CDD) with Reverse Position Specific BLAST analyses. This indicates that the NOV6 sequence has properties similar to those of other proteins known to contain this domain.

TABLE 6I
Domain Analysis of NOV6

gnl|Pfam|pfam01151, GNS1_SUR4, GNS1/SUR4 family.

| | | | | |
|---|---|---|---|---|
| NOV6: | 5 | MNVSHEVNQLFQPYNFELSKDMRPFFEEYWATSFPIALIYLVLIAVGQNYMKERKGFNLQ | 64 | (SEQ ID NO:18) |
| GNS1: | 32 | QVVTYSTVYRFPGKQFEFIYGKTILFESYHAIKIINR--YYIIIFGGQQIMEKYKPFKLK | 89 | (SEQ ID NO:37) |
| NOV6: | 65 | GPLILWSFCLAIFSILGAVRMWGIMGTVLLTGGLKQTVCFINFIDNSTVKFWSWVFLLSK | 124 | |
| GNS1: | 90 | TPLQVHNLFLTSFSILLLLLMVEQLVPSVYAEGLYFSICNSEAWTQVLV-TLYYLNYMSK | 148 | |
| NOV6: | 125 | VIELGDTAFIILRKRPLIFIHWYHHSTVLVYTSFGYKNKVPAGGWFVTMNFGVHAIMYTY | 184 | |
| GNS1: | 149 | FVELIDTVFIVLRKRKLIFLHTYHHGATALLCYHQLKGHTAVGWVPILLNLGVHVLMYWY | 208 | |
| NOV6: | 185 | YTLKAANVKPPKMLPMLITSLQILQMFVGAI-VSILTY---------IWRQDQGCHTTME | 234 | |
| GNS1: | 209 | YFLSALGIR--VWWKMWVTRLQIIQFLLDVIFIYFAVYQKKVHGYLPILPNCGDCQGSWA | 266 | |
| NOV6: | 235 | HLFWSFILYMTYFILFAHFFCQTYIRPKVKAKTK 268 | | |
| GNS1: | 267 | ALALGFAIYTSYLLLFISFYIHAYKKKSNKTVKK 300 | | |

Cold Inducible Glycoprotein 30 is implicated in the thermogenic function of brown adipose tissue of mice. This gene, termed Cig30, is the first mammalian member of a novel gene family comprising several nematode and yeast genes, such as SUR4 and FEN1, mutation of which is associated with highly pleiotropic phenotypes. It codes for a 30-kDa plasma membrane glycoprotein with five putative transmembrane domains. The Cig30 mRNA was readily detected only in brown fat and liver. When animals were exposed to a 3 -day cold stress, the Cig30 expression was selectively elevated in brown fat more than 200-fold. Similar increases were brought about in two other conditions of brown fat recruitment, namely during perinatal development and after cafeteria diet. The magnitude of Cig30 mRNA induction in the cold could be mimicked by chronic norepinephrine treatment in vivo. However, in primary cultures of brown adipocytes, a synergistic action of norepinephrine and dexamethasone was required for full expression of the gene, indicating that both catecholamines and glucocorticoids are required for the induction of Cig30.

GNS1/SUR4 family of eukaryotic integral membrane proteins are evolutionary related, but exact function has not yet clearly been established. The proteins have from 290 to 435 amino acid residues. Structurally, they seem to be formed of three sections: a N-terminal region with two transmembrane domains, a central hydrophilic loop and a C-terminal region that contains from one to three transmembrane domains. As a signature pattern a conserved region that contains three histidines was selected. This region is located in the hydrophilic loop.

The disclosed NOV6 nucleic acid of the invention encoding a Cold inducible glycoprotein 30-like protein includes the nucleic acid whose sequence is provided in Table 6A, C or a fragment thereof. The invention also includes a mutant or variant nucleic acid any of whose bases may be changed from the corresponding base shown in Table 6A or while still encoding a protein that maintains its Cold inducible glycoprotein 30-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV6 protein of the invention includes the Cold inducible glycoprotein 30-like protein whose sequence is provided in Table 6B or 6D. The invention also includes a mutant or variant protein any of whose residues may be changed from the corresponding residue shown in Table 6B or 6D while still encoding a protein that maintains its Cold inducible glycoprotein 30-like activities and physiological functions, or a functional fragment thereof. In the mutant or variant protein, up to about 60% percent of the residues may be so changed.

The invention further encompasses antibodies and antibody fragments, such as F_{ab} or (F_{ab})_{2,} that bind immunospecifically to any of the proteins of the invention.

The above defined information for this invention suggests that this Cold inducible glycoprotein 30-like protein (NOV6) may function as a member of a “Cold inducible glycoprotein 30 family”. Therefore, the NOV6 nucleic acids and proteins identified here may be useful in potential therapeutic applications implicated in (but not limited to) various pathologies and disorders as indicated below. The potential therapeutic applications for this invention include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The NOV6 nucleic acids and proteins of the invention are useful in potential therapeutic applications implicated in cancer including but not limited to various pathologies and disorders as indicated below. For example, a cDNA encoding the Cold inducible glycoprotein 30-like protein (NOV6) may be useful in gene therapy, and the cold inducible glycoprotein 30-like protein (NOV6) may be useful when administered to a subject in need thereof. By way of nonlimiting example, the compositions of the present invention will have efficacy for treatment of patients suffering from Lipoprotein disorder, cirrhosis, and olivopontocerebellar degeneration, hypertrophic obstructive cardiomyopathy, recurrent nonimmune hydrops fetalis and other diseases and/or disorders. The NOV6 nucleic acid encoding the cold inducible glycoprotein 30-like protein of the invention, or fragments thereof, may further be useful in diagnostic applications, wherein the presence or amount of the nucleic acid or the protein are to be assessed.

NOV6 nucleic acids and polypeptides are further useful in the generation of antibodies that bind immuno-specifically to the novel NOV6 substances for use in therapeutic or diagnostic methods. These antibodies may be generated according to methods known in the art, using prediction from hydrophobicity charts, as described in the “Anti-NOVX Antibodies” section below. The disclosed NOV6 protein has multiple hydrophilic regions, each of which can be used as an immunogen. In one embodiment, a contemplated NOV6 epitope is from about amino acids 30 to 55. In another embodiment, a NOV6 epitope is from about amino acids 60 to 140. In additional embodiments, a NOV6 epitope is from about amino acids 160 to 180, from about amino acids 190 to 220, and from about amino acid 230 to 260. These novel proteins can be used in assay systems for functional analysis of various human disorders, which will help in understanding of pathology of the disease and development of new drug targets for various disorders.

NOV7

A disclosed NOV7 nucleic acid of 729 nucleotides (also referred to as SC 122984679_A) encoding a novel matrilin-24like protein is shown in Table 7A. An open reading frame was identified beginning with a ATG initiation codon at nucleotides 5-7 and ending with a TGA codon at nucleotides 701-703. Putative untranslated regions upstream from the initiation codon and downstream from the stop codon are underlined in Table 7A. The start and stop codons are in bold letters.

TABLE 7A
NOV7 Nucleotide Sequence

| | |
|---|---|
| AGCCATGGCCGGCCTCCGAGGGAACGCTGTGGCTGGCCTCCTCTGGATGCTGCTGCTGTGGAGTGGGGGCG | (SEQ ID NO:21) |
| GCGGCTGCCAGGCTCAGCGGGCAGGTTGCAAAAGTGTCCACTACGATCTGGTCTTCCTCCTGGACACCTCC | |
| TCCAGCGTGGGCAAGGAGGACTTTGAGAAGGTCCGGCAGTGGGTGGCCAACCTGGTGGACACCTTCGAGGT | |
| GGGCCCCGACCGCACCCGTGTGGGGGTCGTGCGCTACAGCGACCGGCCCACCACGGCCTTCGAGTTGGGAC | |
| TCTTTGGCTCGCAGGAGGAGGTCAAGGCGGCTGCCCGGCGTCTCGCCTACCACGGGGGCAACACCAACACG | |
| GGAGACGCGCTCCGCTACATCACGGCCCGCAGCTTCTCCCCACACGCCGGCGGCCGCCCCAGGGACCGCGC | |
| CTACAAGCAGGTGGCCATCCTGCTCACCGACGGCCGCAGCCAGGACCTGGTGCTGGACGCCGCGGCGGCAG | |
| CCCACCGCGCTGGCATCCGCATCTTTGCCGTGGGCGTGGGCGAGGCACTCAAGGAGGAGCTGGAGGAGATC | |
| GCCTCAGAGCCCAAGTCCGCCCACGTCTTCCACGTGTCCGACTTCAATGCCATCGACAAGATCCGGGGCAA | |
| GCTGCGGCGCCGTCTTTGTGAAAGTGAGTGCGCCCGGGCCCCATGCGGCCCCTCCCAAGAGTGACGCCCCC | |
| TGCTGAGCCCACGGGAGGG | |

The disclosed NOV7 nucleic acid sequence maps to chromosome 8 and has 408 of 681 bases (59%) identical to a matrilin-4 mRNA from Homo sapiens (GENBANK-ID: AJ007581). The NOV7 nucleic acid disclosed in this invention is expressed in at least the parathyroid gland and the brain.

A disclosed NOV7 protein (SEQ ID NO: 22) encoded by SEQ ID NO: 21 has 232 amino acid residues, and is presented using the one-letter code in Table 7B. Signal P, Psort and/or Hydropathy results predict that NOV7 has a signal peptide, and is likely to be secreted extracellularly with a certainty of 0.3700. In other embodiments NOV7 is also likely to be localized to the endoplasmic reticulum (membrane) with a certainty of 0.1000. The most likely cleavage site is after residue 27 of SEQ ID NO: 22.

TABLE 7B
Encoded NOV7 protein sequence.

| | |
|---|---|
| MAGLRGNAVAGLLWMLLLWSGGGGCQAQRAGCKSVHYDLVFLLDTSSSVGKEDFEKVRQWVANLVDTFEVG | (SEQ ID NO:22) |
| PDRTRVGVVRYSDRPTTAFELGLFGSQEEVKAAARRLAYHGGNTNTGDALRYITARSFSPHAGGRPRDRAY | |
| KQVAILLTDGRSQDLVLDAAAAAHRAGIRIFAVGVGEALKEELEEIASEPKSAHVFHVSDFNAIDKIRGKL | |
| RRRLCESECARAPCGPSQE | |

The disclosed NOV7 amino acid has have 79 of 189 amino acid residues (41%) identical to, and 124 of 189 amino acid residues (65%) similar to, the 313 amino acid residue matrilin-2 protein from Homo sapiens (O00339).

TaqMan expression data for NOV7 is found below is Example 2.

NOV7 also has homology to the amino acid sequences shown in the BLASTP data listed in Table 7C.

TABLE 7C
BLAST results for NOV7

| Gene Index/ | | Length | Identity | Positives | |
|---|---|---|---|---|---|
| Identifier | Protein/Organism | (aa) | (%) | (%) | Expect |
| gi\|4582324\| | Collagen I- | 526 | 43 | 62 | e-39 |
| | like/human | | | | |
| gi\|4505111\| | Matrilin-1/human | 496 | 43 | 62 | e-39 |
| gi\|6857810\| | Matrilin-1/mouse | 500 | 43 | 61 | e-39 |
| gi\|211546\| | Cartilage matrix | 416 | 43 | 61 | e-39 |
| | protein/gallus | | | | |
| | gallus | | | | |
| gi\|115555\| | Matrilin-1/gallus | 493 | 43 | 61 | e-39 |
| | gallus | | | | |

The homology of these sequences is shown graphically in the ClustalW analysis shown in Table 7D.

[image]

[image]

Table 7E lists the domain description from DOMAIN analysis results against NOV7. This indicates that the NOV7 sequence has properties similar to those of other proteins known to contain this domain.

TABLE 7E
Domain Analysis of NOV7

gnl|Pfam|pfam00092, vwa, von Willebrand factor type A domain. (vWF-A)

| | | | | |
|---|---|---|---|---|
| NOV7: | 38 | DLVFLLDTSSSVGKEDFEKVRQWVANLVDTFEVGPDRTRVGVVRYSDRPTTAFELGLFGS | 97 | (SEQ ID NO:22) |
| | | \|+\|\|\|\|\| \| \|+\| ++\|\|+\|+ +\| +\|+ ++\|\|\|+ \|\|\|+\|+\|\|\| \| \|+\| + + | | |
| VWF-A: | 1 | DIVFLLDGSGSIGPQNFERVKDFVERVVERLDIGPDKVRVGLVQYSDNVRTEFKLNDYQN | 60 | (SEQ ID NO:38) |
| NOV7: | 98 | QEEVKAAARRLAYHGGN-TNTGDALRYITARSFSPHAGGRPRDRAYKQVAILLTDGRSQD | 156 | |
| | | ++\|\| \| \|++ \|+\|\| \|\|\|\| \|\|+\|+ \|+ +\| \| +\| ++\|\|\|\|\|\|\|\| | | |
| VWF-A: | 61 | KDEVLQALRKIQYYGGGGTNTGTALQYVVRNLFTEASGSREGAP---KVLVVLTDGRSQD | 117 | |
| NOV7: | 157 | LVL-DAAAAAHRAGIRIFAVGVGEALK-EELEEIASEPKSAHVFHVSDFNAIDKIRGKL | 213 | |
| | | + \| +\|\|+ +\|\|+\|\|\| \| \|\|\| \|\|\|\|+\| \|\|\| \|\|\|\| \|+\| ++ \| | | |
| VWF-A: | 118 | DPIRDVLNELKKAGVNVFAIGVGNADNVEELREIASKPDEQHVFKVSDFEALDTLQELL | 176 | |

Members of the matrilin protein family contain von Willebrand factor A (Vwfa) domains, epidermal growth factor (EGF)-like motifs, and a coiled-coil alpha-helical module. Matrilin-2 and -4 occur in a wide variety of extracellular matrices. Wagener et al. (1998) isolated cDNAs encoding mouse matrilin4. By searching sequence databases with the mouse matrilin-4 cDNA, Wagener et al. (1998) identified genomic and cDNA clones corresponding to the human homolog. The human matrilin-4 gene contains 10 exons and spans approximately 12 kb. Alternative splicing leads to 3 different transcripts encoding protein isoforms that all contain a putative signal peptide, 2 vWFA-like domains, and the coiled-coil region. The isoforms differ in that they include either 1, 2, or 3 EGF-like domains. There are 4 EGF-like domains in mouse matrilin-4, but the first EGF-like domain in the human protein is not expressed. RT-PCR detected expression of human matrilin4 in lung and placenta and in 2 cell lines. Wagener et al. (1998) noted that Jay et al. (1997) isolated a matrilin4 cDNA, designated HE6WCR54, in a project designed to identify genes expressed during early human development

A mouse cDNA encoding a novel member of the von Willebrand factor type A-like module superfamily was cloned. The protein precursor of 956 amino acids consists of a putative signal peptide, two von Willebrand factor type A-like domains connected by 10 epidermal growth factor-like modules, a potential oligomerization domain, and a unique segment, and it contains potential N-glycosylation sites. A sequence similarity search indicated the closest relation to the trimeric cartilage matrix protein (CMP). Since they constitute a novel protein family, we introduce the term matrilin-2 for the new protein, reserving matrilin-1 as an alternative name for CMP. A 3. 9-kilobase matrilin-2 mRNA was detected in a variety of mouse organs, including calvaria, uterus, heart, and brain, as well as fibroblast and osteoblast cell lines. Expressed human and rat cDNA sequence tags indicate a high degree of interspecies conservation. A group of 120-150-kDa bands was, after reduction, recognized specifically with an antiserum against the matrilin-2-glutathione S-transferase fusion protein in media of the matrilin-2-expressing cell lines. Assuming glycosylation, this agrees well with the predicted minimum Mr of the mature protein (104,300). Immunolocalization of matrilin-2 in developing skeletal elements showed reactivity in the perichondrium and the osteoblast layer of trabecular bone. CMP binds both collagen fibrils and aggrecan, and because of the similar structure and complementary expression pattern, matrilin-2 is likely to perform similar functions in the extracellular matrix assembly of other tissues.

The von Willebrand factor is a large multimeric glycoprotein found in blood plasma. Mutant forms are involved in the aetiology of bleeding disorders. In von Willebrand factor, the type A domain (vWF) is the prototype for a protein superfamily. The vWF domain is found in various plasma proteins: complement factors B, C2, CR3 and CR4; the integrins (I-domains); collagen types VI, VII, XII and XIV; and other extracellular proteins. Proteins that incorporate vWF domains participate in numerous biological events (e.g., cell adhesion, migration, homing, pattern formation, and signal transduction), involving interaction with a large array of ligands. Secondary structure prediction from 75 aligned vWF sequences has revealed a largely alternating sequence of alpha-helices and beta-strands. Fold recognition algorithms were used to score sequence compatibility with a library of known structures: the vWF domain fold was predicted to be a doubly-wound, open, twisted beta-sheet flanked by alpha-helices. 3D structures have been determined for the I-domains of integrins CD11b (with bound magnesium) and CD11a (with bound manganese). The domain adopts a classic alpha/beta Rossmann fold and contains an unusual metal ion coordination site at its surface. It has been suggested that this site represents a general metal ion-dependent adhesion site (MIDAS) for binding protein ligands. The residues constituting the MIDAS motif in the CD11b and CD11a I-domains are completely conserved, but the manner in which the metal ion is coordinated differs slightly.

The disclosed NOV7 nucleic acid of the invention encoding a Matrilin-2-like protein includes the nucleic acid whose sequence is provided in Table 7A or a fragment thereof. The invention also includes a mutant or variant nucleic acid any of whose bases may be changed from the corresponding base shown in Table 7A while still encoding a protein that maintains its Matrilin-2-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV7 protein of the invention includes the Matrilin-2-like protein whose sequence is provided in Table 7B. The invention also includes a mutant or variant protein any of whose residues may be changed from the corresponding residue shown in Table 7B while still encoding a protein that maintains its Matrilin-2-like activities and physiological functions, or a functional fragment thereof. In the mutant or variant protein, up to about 13% percent of the residues may be so changed.

The protein similarity information, expression pattern, and map location for the Serotonin receptor-like protein and nucleic acid (NOV7) disclosed herein suggest that NOV7 may have important structural and/or physiological functions characteristic of the serotonin receptor-like family. Therefore, the NOV7 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV7 nucleic acids and proteins of the invention are useful in potential diagnostic and therapeutic applications implicated in various diseases and disorders described below. For example, the compositions of the present invention will have efficacy for treatment of patients suffering from fibrosarcoma, multiple sclerosis, chondrodysplasias: hypochondroplasia, achondroplasia, autosomal dominant SED tarda, and multiple epiphyseal dysplasia, polychondritis, and/or other pathologies. The NOV7 nucleic acid, or fragments thereof, may further be useful in diagnostic applications, wherein the presence or amount of the nucleic acid or the protein are to be assessed.

NOV7 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV7 protein have multiple hydrophilic regions, each of which can be used as an immunogen. In one embodiment, contemplated NOV7 epitope is from about amino acids 5 to 25. In another embodiment, a NOV7 epitope is from about amino acids 40 to 50. In additional embodiments, NOV7 epitopes are from about amino acids 145 to 155, and from about amino acids 160 to 180. This novel protein also has value in development of powerful assay system for functional analysis of various human disorders, which will help in understanding of pathology of the disease and development of new drug targets for various disorders.

NOV8

A disclosed NOV8 nucleic acid of 682 nucleotides (also referred to as SC65666665_A) encoding a novel CD53 (leuococyte surface antigen)-like protein is shown in Table 8A. An open reading frame was identified beginning with a ATG initiation codon at nucleotides 2-4 and ending with a TGA codon at nucleotides 655-657. Putative untranslated regions upstream from the initiation codon and downstream from the stop codon are underlined in Table 8A. The start and stop codons are in bold letters.

TABLE 8A
NOV8 Nucleotide Sequence

(SEQ ID NO:23)

CATGGGCACAAGTAGCTTGAAATTATGGAAGTATGTCCTGTCTTTCTTTCTTTTCTTTCTTTCTTTCTTA
CTCTCTCTCACTTTCGGGATGTACCCGCTGATCCACAACAGTTTGGGAGTGCTCTTCCATAAGCTCCCCT
CCCTCATGCCGGGCAATGTGCTTGTCATCGTGGTCTCCATTATCACGGTGGTTGCCTTCCTGGGCTGCAT
AGGTTCTGTCAAGAAAAACAGGTGCCTGCTTATGTCCTTGTTCATTCTGCTGCCGGTTATCCTCCTTGCT
GAGGTGATCTTGGCCATCCTGCACTTTGTTTACGAACGGAAGCTGAATGTATACGTAGCTGAGGGCCTGA
CGGACAGCATCTACCATTACCACTGGGACAACAGCACCAAGGCGATGTGGGACTCCATCCAGTCATTCTG
CACTTGCTGTGGCGTAAATGGCATGAGTGATTGGTCCAGCGGACCGCAAGCATCTTGCCCCTCAGATCCA
AAAGTGAAAGGGTGCTATGCAAAAGCGAGACTGTGGTTTCACGCCAATTTCCTGTATATCAGAATCATCA
CCATCTGTGTAATATGTGCAATCCAGGTGGTGAGGATGTCCTTTGCACTGACCCCAAACAGCCAGATTGA
TAAACCAGTCAGGCCCTGGGGGTGTGACCTGCCAACTGCCCTGTGCTGGGGA

The disclosed NOV8 nucleic acid sequence maps to chromosome 1 and has 569 of 685 bases 83%) identical to a Human CD53 glycoprotein mRNA (GENBANK-ID: M37033). The NOV8 nucleic acid disclosed in this invention is expressed in at substantia nigra, B cells, monocytes, macrophages, neutrophils, single (cd4 or cd8) positive thymocytes, and peripheral T cells.

A disclosed NOV8 protein (SEQ ID NO: 24) encoded by SEQ ID NO: 23 has 218 amino acid residues, and is presented using the one-letter code in Table 8B. Signal P, Psort and/or Hydropathy results predict that NOV8 has a signal peptide, and is likely to be localized to the plasma membrane with a certainty of 0.6400. In other embodiments NOV8 is also likely to be localized to the Golgi body with a certainty of 0.4600, or to the endoplasmic reticulum (membrane) with a certainty of 0.3700. The most likely cleavage site is after residue 29 of SEQ ID NO: 24.

TABLE 8B
Encoded NOV8 protein sequence.

(SEQ ID NO:24)

MGTSSLKLWKYVLSFFLFFLSFLLSLTFGMYPLIHNSLGVLFHKLPSLMPGNVLVIVVSIITVVAFLGCI
GSVKKNRCLLMSLFILLPVILLAEVILAILHFVYERKLNVYVAEGLTDSIYHYHWDNSTKAMWDSIQSFC
TCCGVNGMSDWSSGPQASCPSDPKVKGCYAKARLWFHANFLYIRIITICVICAIQVVRMSFALTPNSQID
KTSQALGV

The disclosed NOV8 amino acid has 161 of 218 amino acid residues (73%) identical to, and 181 of 218 amino acid residues (82%) similar to, the 219 amino acid residue leukocyte surface antigen cd53 protein from Homo sapiens (Human) (P19397).

TaqMan expression data for NOV8 is found below is Example 2. SNP data for NOV8 is found below in Example 3.

NOV8 also has homology to the amino acid sequences shown in the BLASTP data listed in Table 8C.

TABLE 8C
BLAST results for NOV8

| Gene Index/ | | Length | Identity | Positives | |
|---|---|---|---|---|---|
| Identifier | Protein/Organism | (aa) | (%) | (%) | Expect |
| gi\|10834972\| | CD-53/human | 219 | 73 | 82 | e-72 |
| gi\|6978633\| | OX-44/rat | 219 | 66 | 76 | e-64 |
| gi\|6671712\| | CD-53/mouse | 219 | 66 | 76 | e-64 |
| gi\|5729941\| | NET-5 | 239 | 32 | 52 | e-17 |
| | tetraspan/human | | | | |
| gi\|15307298\| | Tetraspan 4 | 175 | 35 | 56 | e-15 |
| | protein/human | | | | |

The homology of these sequences is shown graphically in the ClustalW analysis shown in Table 8D.

[image]

[image]

Table 8E lists the domain description from DOMAIN analysis results against NOV8. This indicates that the NOV8 sequence has properties similar to those of other proteins known to contain this domain.

TABLE 8E
Domain Analysis of NOV8

gnl|Pfam|pfam00335, transmembrane4, Tetraspanin family. (Tetra)

| (SEQ ID NO: 24) | | | |
|---|---|---|---|
| (SEQ ID NO: 39) | | | |
| NOV8: | 36 | NSLGVLFHKLPSLMPGNVLVIVVSIITVVAFLGCIGSVKKNRCLLMSLFILLPVILLAEV | 95 |
| | | +S L L SL+ VL+ V +i+ +V FLGC G+++++RCLL F+ L +I + EV | |
| Tetra: | 30 | SSFSELLGSLSSLVAAYVLIAVGAILFLVGFLGCCGAIRESRCLLGLYFVFLLLIFILEV | 89 |
| NOV8: | 96 | ILAILHFVYERKLNVYYVAEGLTDSIYH-YHWDNSTKAMWDSIQSFCTCCGVNGMSDW--- | 151 |
| | | IL FV+ KL + E L ++I + Y D + WD +Q CCGVNG +DW | |
| Tetra: | 90 | AAGILAFVFRDKLESSLNESLKNAIKNYYDTDPDERNAWDKLQEQFKCCGVNGYTDWFDS | 149 |
| NOV8: | 152 | ---SSGPQASCPSDPK VKGCYAKARLWFHANFL | 181 |
| | | S+G SC + +GC K W N L | |
| Tetra: | 150 | QWFSNGVPFSCCNPSVSCNSAQDEEDTIYQEGCLEKLLEWLEENLL | 195 |

CD53 is an N-glycosylated pan-leucocyte antigen of 35-42,000 Mr. The sequence of the CD53 polypeptide deduced from a cDNA clone is 219 amino acids in length. It appears to lack a conventional leader sequence because the deduced NH2-terminal amino acid sequence is very similar to the rat MRC OX-44 and human CD37 antigens. The CD53 molecule is likely to consist of four transmembrane regions and a major extracellular hydrophilic loop containing two potential N-glycosylation sites. It is suggested that the CD53 glycoprotein is the true human homologue of the rat OX-44 antigen, rather than the CD37 antigen of more restricted expression and lower NH2-terminal sequence similarity to OX-44. CD53 is a human cell-surface Ag expressed exclusively by nucleated cells of hemopoietic origin. CD53 transcripts increase in prevalence after mitogenic stimulation, suggesting that the protein may be involved in the transport of factors essential for cell proliferation. These proteins are also postulated to be involved in signal transduction.

CD37, CD53, CD83 (HB15), CDw84, CD85, CD86 and R2 leukocyte surface antigens are members of a novel family of structurally related proteins. They all have four transmembrane-spanning domains with a single major extracellular loop. These proteins are all type II membrane proteins: they contain an N-terminal transmembrane (TM) domain, which acts both as a signal sequence and a membrane anchor, and 3 additional TM regions (hence the name ‘TM4’). The sequences contain a number of conserved cysteine residues. The CD37 is expressed on B cells and on a subpopulation of T cells. The CD53 is known as a panleukocyte marker. The R2 protein is an activation antigen of T cells. CD83 (HB15) is a marker for human interdigitating reticulum cells, circulating dendritic cells and Langerhans cells. CDw84 and CD85 are new B cell-associated molecules that are also expressed by monocytes. CD86 is a new B cell activation antigen.

The CD37, CD53, and R2 genes were assigned with the help of human/rodent somatic cell hybrids and human-specific probes to human chromosomes 19, 1, and 11, respectively. For the regional assignment, various deletion hybrids were used to map CD37 to 19p 13-q13.4, CD53to 1p12-p31,and R2to 11p12.

The disclosed NOV8 nucleic acid of the invention encoding a CD-53-like protein includes the nucleic acid whose sequence is provided in Table 8A or a fragment thereof. The invention also includes a mutant or variant nucleic acid any of whose bases may be changed from the corresponding base shown in Table 8A while still encoding a protein that maintains its CD-53-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV8 protein of the invention includes the CD-53-like protein whose sequence is provided in Table 8B. The invention also includes a mutant or variant protein any of whose residues may be changed from the corresponding residue shown in Table 8B while still encoding a protein that maintains its CD-53-like activities and physiological functions, or a functional fragment thereof. In the mutant or variant protein, up to about 13% percent of the residues may be so changed.

The protein similarity information, expression pattern, and map location for the Serotonin receptor-like protein and nucleic acid (NOV8) disclosed herein suggest that NOV8 may have important structural and/or physiological functions characteristic of the serotonin receptor-like family. Therefore, the NOV8 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV8 nucleic acids and proteins of the invention are useful in potential diagnostic and therapeutic applications implicated in various diseases and disorders described below. For example, the compositions of the present invention will have efficacy for treatment of patients suffering cancer, autoimmune disease, and infectious diseases. These diseases include but are not limited to Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection, Endocrine dysfunctions, Diabetes, obesity, Growth and reproductive disorders, Myasthenia gravis, Hemophilia, hypercoagulation, Idiopathic thrombocytopenic purpura, autoimmume disease, allergies, immunodeficiencies, transplantation, Graft vesus host disease (GVHD), Anemia, Ataxia-telangiectasia, Autoimmume disease, Hypercoagulation, Idiopathic thrombocytopenic purpura, Lymphedema, Lymphaedema, and cancers including but not limited to bone cancer, brain cancer, and liver cancer and/or other pathologies. The NOV8 nucleic acid, or fragments thereof, may further be useful in diagnostic applications, wherein the presence or amount of the nucleic acid or the protein are to be assessed.

NOV8 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV8 protein have multiple hydrophilic regions, each of which can be used as an immunogen. In one embodiment, contemplated NOV8 epitope is from about amino acids 10 to 50. In another embodiment, a NOV8 epitope is from about amino acids 50 to 110. In additional embodiments, NOV8 epitopes are from about amino acids 140 to 150, and from about amino acids 165 to 200. This novel protein also has value in development of powerful assay system for functional analysis of various human disorders, which will help in understanding of pathology of the disease and development of new drug targets for various disorders.

NOV9

A disclosed NOV9 nucleic acid of 1580 nucleotides (also referred to as GM358d14_A) encoding a novel tyrosine kinase-like protein is shown in Table 9A. An open reading frame was identified beginning with a ATG initiation codon at nucleotides 18-20 and ending with a TGA codon at nucleotides 1578-1580. Putative untranslated regions upstream from the initiation codon and downstream from the stop codon are underlined in Table 9A. The start and stop codons are in bold letters.

TABLE 9A
NOV9 Nucleotide Sequence

(SEQ ID NO:25)

CGTGGTGACCCCGGGGATGGAGCCGTTCCTCAGGAGGCGGCTGGCCTTCCTGTCCTTCTTCTGGGACAAG
ATCTCGCCGGCGGGCGGCGAGCCGGACCATGGCACCCCCGGGTCCCTGGACCCCAACACTGACCCGTGCC
CACGCTCCCCGCCGAGCCTTCAGCCCCTTCCCTCAGCTCTTCCTTGCGCTCTATGACTTCACGGCGGT
GTGGCGGGGAGCTGAGTGTCCGCCGCGGGGACAGGCTCTGTGCCCTCGAAGAGGGGGGCGGCTACATCTTC
GCACGCAGGCTTTCGGGCCAGCCCAGCGCCGGGCTCGTGCCCATCACCCACGTGGCCAAGGCTTCTCCTGA
GACGCTCTCAGACCAACCGCCTGCTGTTTGCAGCTGGTACTTTAGCGGGGTCAGTCGGACCCAGGCACAGC
AGCTGCTCCTCTCCCCACCCAACGAACCAGGGGCCTTCCTCATCCGGCCCAGCGAGAGCAGCCTCGGGGGC
TACTCACTGTCAGTCCGGGCCCAGGCCAAGGTCTGCCACTACCGGGTCTCCATGGCAGCTGATGGCAGCCT
CTACCTGCAGAAGGGACGGCTCTTTCCCGGCCTGGAGGAGCTGCTCACCTACTACAAGGCCAACTGGAAGC
TGATCCAGAACCCCCTGCTGCAGCCCTGCATGCCCCGGTGGGCCTGCCCTGCCCACCCTCCCTGCAGAAG
GCCCTGCGGCAGGACGTGTGGGAGCGGCCACACTCCGAATTCGCCCTTGGGAGGAAGCTGGGTGAAGGCTA
CTTTGGGGAGGTGTGGGAAGGCCTGTGGCTGGGCTCCCTGCCCGTGGCGATCAAGGTCATCAAGTCAGCCA
ACATGAAGCTCACTGACCTCGCCAAGGAGATCCAGACACTGAAGGGCCTGCGGCACGAGCGGCTCATCCGG
CTGCACGCAGTGTGCTCGGGCGGGGAGCCTGTGTSCATCCTCACGGAACTCATGCGCAAGGGGAACCTGCA
GGCCTTCCTGGGCAGTGGCTCTGCTCCACTCCCCTCTGCAGACTCTGATGAGAAAGTCCTGCCCGTTTCGG
AGCTGCTGGACATCGCCTGGCAGGTGGCTGAGGGCATGTGTTACCTGGAGTCGCAGAATTACATCCACCGG
GACCTGGCCGCCAGGAACATCCTCGTCGGGGAAAACACCCTCTGCAAAGTTGGGGACTTCGGGTTAGCCAG
GCTTATCAAGGTAGGGCCCTCAGAGGGCCAGGACGACATCTACTCCCCGAGCAGCAGCTCCAAGATCCCGG
TCAAGTGGACAGCGCCTGAGGCGGCCAATTATCGTGTCTTCTCCCAGAAGTCAGACGTCTGGTCCTTCGGC
GTCCTGCTGCACGAGGTTTTCACCTATGGCCAGTGTCCCTATGAAGGTGGGATGACCAACCAGAGACGCT
GCAGCAGATCATGCGAGGGTACCGGCTGCCGCGCCCGGCTGCCTGCCCGACGGAGGTCTACTTGCTCATGC
TGGAGTGCTGGAGGAGCAGCCCCGAGGAACGGCCCTCCTTCGCCACGCTGCGGGAGAAGCTGCACGCCATC
CACAGATGCCACCCCTGA

The disclosed NOV9 nucleic acid sequence maps to chromosome 20 and has 470 of 673 bases (69%) identical to a tyrosine-specific protein kinase mRNA from Mus musculus (GENBANK-ID: D26186). The NOV9 nucleic acid disclosed in this invention is expressed in at least the kidney.

A disclosed NOV9 protein (SEQ ID NO: 26) encoded by SEQ ID NO: 25 has 520 amino acid residues, and is presented using the one-letter code in Table 9B. Signal P, Psort and/or Hydropathy results predict that NOV9 has a signal peptide, and is likely to be localized to the mitochondrial matrix space with a certainty of 0.6917. In other embodiments NOV9 is also likely to be localized to the microbody with a certainty of 0.4434, or to the mitochondrial inner membrane with a certainty of 0.3782. The most likely cleavage site is after residue 24 of SEQ ID NO: 26.

TABLE 9B
Encoded NOV9 protein sequence.

| | |
|---|---|
| MEPFLRRRLAFLSFFWDKIWPAGGEPDHGTPGSLDPNTDPVPTLPAEPCSPFPQLFLALYDFTARCG | (SEQ ID NO:26) |
| GELSVRRGDRLCALEEGGGYIFARRLSGQPSAGLVPITHVAKASPETLSDQPPAVCSWYFSGVSRT | |
| QAQQLLLSPPNEPGAFLIRPSESSLGGYSLSVRAQAKVCHYRVSMAADGSLYLQKGRLFPGLEELL | |
| TYYKANWKLIQNPLLQPCMPQVGLPCPPSLQKALRQDVWERPHSEFALGRKLGEGYFGEVWEGL | |
| WLGSLPVAIKVIKSANMKLTDLAKEIQTLKGLRHERLIRLHAVCSGGEPVYILTELMRKGNLQAFL | |
| GSGSAPLPSADSDEKVLPVSELLDIAWQVAEGMCYLESQNYIHRDLAARNILVGENTLCKVGDFG | |
| LARLIKVGPSEGQDDIYSPSSSSKIPVKWTAPEAANYRVFSQKSDVWSFGVLLHEVFTYGQCPYEG | |
| GMTNHETLQQIMRGYRLPRPAACPTEVYLLMLECWRSSPEERPSFATLREKLHAIHRCHP | |

The disclosed NOV9 amino acid has 238 of 331 amino acid residues (71%) identical to, and 265 of 331 amino acid residues (80%) similar to, the 496 amino acid residue protein-tyrosine kinase (EC 2.7.1.112) Srm, nonreceptor type protein from Mus musculus (A56040).

TaqMan expression data for NOV9 is found below is Example 2. SNP data for NOV9 is found below in Example 3.

NOV9 also has homology to the amino acid sequences shown in the BLASTP data listed in Table 9C.

TABLE 9C
BLAST results for NOV9

| | | | | | |
|---|---|---|---|---|---|
| Gene Index/ | | Length | Identity | Positives | |
| Identifier | Protein/Organism | (aa) | (%) | (%) | Expect |

| | | | | | |
|---|---|---|---|---|---|
| gi\|11137518\| | Novel tyrosine | 488 | 88 | 89 | 0.0 |
| | kinase/human | | | | |
| gi\|14330032\| | Src-related | 496 | 70 | 78 | 0.0 |
| | kinase/mouse | | | | |
| gi\|6755658\| | Src-related | 496 | 70 | 78 | 0.0 |
| | kinase/mouse | | | | |
| gi\|2499673\| | SRM kinase/ | 496 | 70 | 78 | 0.0 |
| | mouse | | | | |
| gi\|5174647\| | PTK6 kinase/ | 451 | 49 | 60 | e-107 |
| | human | | | | |

The homology of these sequences is shown graphically in the ClustalW analysis shown in Table 9D.

NOV9 (SEQ ID NO: 26)

gi|111375181 |(SEQ ID NO: 121)

gi|14330032 |(SEQ ID NO: 122)

gi|67556581 |(SEQ ID NO: 123)

gi|24996731 |(SEQ ID NO: 124)

gi|51746471 |(SEQ ID NO: 125)

[image]

[image]

[image]

Table 9E-F lists the domain description from DOMAIN analysis results against NOV9. This indicates that the NOV9 sequence has properties similar to those of other proteins known to contain this domain.

TABLE 9E
Domain Analysis of NOV9

gnl|Smart|smart002l9, TyrKc, Tyrosine kinase, catalytic domain;
Phosphotransferases. Tyrosine-specific kinase subfamily.

| (SEQ ID NO: 26) | | | |
|---|---|---|---|
| (SEQ ID NO: 40) | | | |
| NOV9: | 247 | LGRKLGEGYFGEVWEGLWLGS----LPVAIKVIKSANMK--LTDLAKEIQTLKGLRHERL | 300 |
| | | \|\|+\|\|\|\|\| \|\|\|\|++\| \| + \|\|+\| +\| + + + +\| + ++ \| \| + | |
| TyrKc: | 3 | LGKKLGEGAFGEVYKGTLKGKGGVEVEVAVKTLKEDASEQQIEEFLREARLMRKLDHPNI | 62 |
| NOV9: | 301 | IRLHAVCSGGEPVYILTELMRKGNLQAFLGSGSAPLPSADSDEKVLPVSELLDIAWQVAE | 360 |
| | | ++\| \|\|+ \|\|+ \|+ \| \| \|+\| +\| + \| \| +\|+\|\| \| \|+\| | |
| TyrKc: | 63 | VKLLGVCTEEEPLMIVMEYMEGGDLLDYL---------RKNRPKELSLSDLLSFALQIAR | 113 |
| NOV9: | 361 | GMCYLESQNYIHRDLAARNILVGENTLCKVGDFGLARLIKVGPSEGQDDIYSPSSSSKIP | 420 |
| | | \|\| \|\|\|\|+\|++\|\|\|\|\|\|\|\| \|\|\|\|\| \|+ \|\|\|\|\|\| + \|\| \| \| ++\| | |
| TyrKc: | 114 | GMEYLESKNFVHRDLAARNCLVGENKTVKIADFGLARDL------YDDDYYRKKKSPRLP | 167 |
| NOV9: | 421 | VKWTAPEAANYRVFSQKSDVWSFGVLLHEVFTYGQCPYEGGMTNHETLQQIMRGYRLPRP | 480 |
| | | ++\| \|\|\|+ \|+ \|+\|\| \|+ \|\| \|\|+\| \| \|+ + +\|\|\|\|\|+\| | |
| TyrKc: | 168 | IRWMAPESLKDGKFTSKSDVWSFGVLLWEIFTLGESPYP-GMSNEEVLEYLKKGYRLPQP | 226 |
| NOV9: | 481 | AACPTEVYLLMLECWRSSPEERPSFATLREKL | 512 |
| | | \|\| \|+\| \|\|\|+\|\| \|\|+\|\|+\|+ \| \|+\| | |
| TyrKc: | 227 | PNCPDEIYDLMLQCWAEDPEDRPTFSELVERL | 258 |

TABLE 9F
Domain Analysis of NOV9

PFAM00069; pkinase, Protein kinase domain (PrKin)

| (SEQ ID 50: 26) | | | |
|---|---|---|---|
| (SEQ ID NO: 41) | | | |
| NOV9: | 245 | FALGRKLGEGYFGEVWEGLWLGS-LPVAIKVIKSANMKLT--DLAKEIQTLKGLRHERLI | 301 |
| | | + \|\| \|\|\| \| \|\|+\|++\| + \|\|\|\|++\| ++ +\|\|\| \|+ \| \| ++ | |
| PrKin: | 1 | YELGEKLGSGAFGKVVKGKHKDTGEIVAIKILKKRSLSEKKKRFLREIQILRRLSHPNIV | 60 |
| NOV9: | 302 | RLHAVCSGGEPVYILTELMRKGNLQAFLGSGSAPLPSADSDEKVLPVSELLDIAWQVAEG | 361 |
| | | \|\| \| + +\|++ \| \| \|+\| +\| \| \| \|\| \|+ \| | |
| PrKin: | 61 | RLLGVFEEDDHLYLVMEYMEGGDLFDYLRRNGLLLSEK----------EAKKIALQILRG | 110 |
| NOV9: | 362 | MCYLESQNYIHRDLAARNILVGENTLCKVGDFGLARLIKVGPSEGQDDIYSPSSSSKIPV | 421 |
| | | + \|\| \|+ +\|\|\|\| \|\|\|+ \|\| \|+ \|\|\|\|\|\| ++ \| ++ | |
| PrKin: | 111 | LEYLHSRGIVHRDLKPENILLDENGTVKIADFGLARKLE-------SSSYEKLTTFVGTP | 163 |
| NOV9: | 422 | KWTAPEAANYRVFSQKSDVWSFGVLLHEVFTYGQCPYEGGMTNHETLQQIMRG-YRLPRP | 480 |
| | | ++ \|\|\| \| +\| \|\|\|\| \|\|+\|+\|+ \| \|+ \| \| + \| \|\|\| \| | |
| PrKin: | 164 | EYMAPEVLEGRGYSSKVDVWSLGVILYELLTGKL-PFPGIDPLEELFRIKERPRLRLPLP | 222 |
| NOV9: | 481 | AACPTEVYLLMLECWRSSPEERPSFATLRE | 510 |
| | | \| \|+ \|+ +\| \|\|+\|\|+ + | |
| PrKin: | 223 | PNCSEELKDLIKKCLNKDPEKRPTAKEILN | 252 |

In the mouse, Klages et al. (1994) reported the molecular cloning and preliminary functional characterization of a nonreceptor protein tyrosine kinase (PTK) that is related to CSK (124095). This PTK, designated Ctk for CSK-type protein-tyrosine kinase, was found to be a 52-kD protein expressed primarily in brain and predicted to be structurally similar to CSK. Klages et al. (1994) found that, like CSK, Ctk can phosphorylate members of the SRC family of PTKs at the regulatory tyrosine residue. Thus, Ctk and CSK define a family of kinases that phosphorylate carboxy-terminal regulatory tyrosine residues.

Protein-tyrosine kinases play major roles in signal transduction pathways. Bennett et al. (1994) cloned a novel tyrosine kinase, termed megakaryoctye-associated tyrosine kinase (MATK), from a human megakaryocyte cDNA library using degenerate PCR. The MATK cDNA encodes a 527-amino acid protein that shows 50% amino acid identity to CSK and has the structural features of the CSK subfamily: SRC homology SH (2) and SH3 domains, a catalytic domain, a unique N terminus, lack of myristylation signals, lack of a negative regulatory phosphorylation site, and lack of an autophosphorylation site. Bennett et al. (1994) localized the MATK protein to the cytoplasm of megakaryocytic cells using immunofluorescence and immunoblot analysis of subcellular fractions. They showed by Northern blotting that the MATK gene is expressed abundantly in megakaryocytes and at a lower level in adult brain as a 2.3-kb transcript; it was not detectably expressed in any other examined tissue. Bennett et al. (1994) found that MATK expression is upregulated in megakaryocytic cells that are induced to differentiate by phorbol ester. They suggested that MATK functions in signal transduction pathways that are important in megakaryocyte growth and/or differentiation.

Avraham et al. (1995) showed that MATK can phosphorylate the SRC (176947) protein in vitro. Sakano et al. (1994) cloned the MATK cDNA, named HYL by them, and localized the gene to 19p 13.3 using fluorescence in situ hybridization. Avraham et al. (1995) mapped the MATK gene to chromosome 19 using somatic cell hybrids and found that the murine Matk gene maps within a region of synteny on chromosome 10. Zrihan-Licht et al. (1997) reported that MATK is expressed in human breast cancer but not in the adjacent normal breast tissues, suggesting that MATK might be involved in signaling in some cases of breast cancer. Zrihan-Licht et al. (1997) demonstrated that MATK interacts with ErbB-2 (164870) in vivo upon heregulin stimulation and that this interaction occurs via the SH2 domain of MATK.

Dymecki et al. (1990) reported the specific expression of a novel tyrosine kinase gene, Blk, in B lymphocytes of the mouse. They demonstrated that the gene is a member of the SRC family of protooncogenes and concluded, on the basis of its preferential expression in B-lymphoid cells, that it functions in a signal transductory pathway specific to this lineage. By a study of intersubspecies backcrosses, Kozak et al. (1991) mapped the gene to mouse chromosome 14. Islam et al. (1995) reported the molecular cloning of the human BLK gene and its expression. By fluorescence in situ hybridization and somatic cell hybrid analysis, they mapped BLK to 8p23-p22. This region is homologous to the region of chromosome 14 carrying the mouse blk locus. The BLK gene is a nonreceptor protein tyrosine kinase with a calculated molecular mass of about 58 kD. It has an overall amino acid identity of approximately 87% to the mouse Blk, however in the unique domain, there is only 58% homology and an insertion of 6 amino acids in the N-terminal region. The nature of this insertion suggested a functional role in membrane attachment. Islam et al. (1995) did not detect the BLK transcript in non lymphoid tissues examined. In contrast, expression of murine Blk in plasma cells and T lymphocytes had not been reported. They saw transcripts in human embryonic liver as early as 7.5 weeks of gestation, before the rearrangement of the immunoglobulin heavy-chain gene locus. Furthermore, they detected transcripts in human thymocytes and not in mature T cells. Southern blot analysis revealed polymorphism of this gene in a Caucasian population but not in a Gambian population, indicating a recent origin of this polymorphism. Expression of BLK in immature T cells suggested that it may play an important role in thymopoiesis. Drebin et al. (1995) likewise cloned the human homolog of murine blk and mapped it to 8p23-p22 by isotopic in situ hybridization. The protein predicted by the open reading frame of the cDNA had 505 amino acids with SH3, SH2, and catalytic domains that contained consensus sequences of the SRC protein tyrosine kinase family (see 190090). Like the murine blk gene, human BLK is expressed only in B lymphocytes.

The disclosed NOV9 nucleic acid of the invention encoding a tyrosine kinase-like protein includes the nucleic acid whose sequence is provided in Table 9A or a fragment thereof. The invention also includes a mutant or variant nucleic acid any of whose bases may be changed from the corresponding base shown in Table 9A while still encoding a protein that maintains its tyrosine kinase-like activities and physiological functions, or a fragment of such a nucleic acid. The invention further includes nucleic acids whose sequences are complementary to those just described, including nucleic acid fragments that are complementary to any of the nucleic acids just described. The invention additionally includes nucleic acids or nucleic acid fragments, or complements thereto, whose structures include chemical modifications. Such modifications include, by way of nonlimiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject. In the mutant or variant nucleic acids, and their complements, up to about 10% percent of the bases may be so changed.

The disclosed NOV9 protein of the invention includes the tyrosine kinase -like protein whose sequence is provided in Table 9B. The invention also includes a mutant or variant protein any of whose residues may be changed from the corresponding residue shown in Table 9B while still encoding a protein that maintains its tyrosine kinase4like activities and physiological functions, or a functional fragment thereof. In the mutant or variant protein, up to about 13% percent of the residues may be so changed.

The protein similarity information, expression pattern, and map location for the tyrosine kinase-like protein and nucleic acid (NOV9) disclosed herein suggest that NOV9 may have important structural and/or physiological functions characteristic of the serotonin receptor-like family. Therefore, the NOV9 nucleic acids and proteins of the invention are useful in potential diagnostic and therapeutic applications. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo.

The NOV9 nucleic acids and proteins of the invention are useful in potential diagnostic and therapeutic applications implicated in various diseases and disorders described below. For example, the compositions of the present invention will have efficacy for treatment of patients suffering cancer, autoimmune disease, and infectious diseases. These diseases include but are not limited to Von Hippel-Lindau (VHL) syndrome, Alzheimer's disease, Stroke, Tuberous sclerosis, hypercalceimia, Parkinson's disease, Huntington's disease, Cerebral palsy, Epilepsy, Lesch-Nyhan syndrome, Multiple sclerosis, Leukodystrophies, Behavioral disorders, Addiction, Anxiety, Pain, Neuroprotection, Endocrine dysfunctions, Diabetes, obesity, Growth and reproductive disorders, Myasthenia gravis, Hemophilia, hypercoagulation, Idiopathic thrombocytopenic purpura, autoimmume disease, allergies, immunodeficiencies, transplantation, Graft vesus host disease (GVHD), Anemia, Ataxia-telangiectasia, Autoimmume disease, Hypercoagulation, Idiopathic thrombocytopenic purpura, Lymphedema, Lymphaedema, and cancers including but not limited to bone cancer, brain cancer, and liver cancer and/or other pathologies. The NOV9 nucleic acid, or fragments thereof, may further be useful in diagnostic applications, wherein the presence or amount of the nucleic acid or the protein are to be assessed.

NOV9 nucleic acids and polypeptides are further useful in the generation of antibodies that bind immunospecifically to the novel substances of the invention for use in therapeutic or diagnostic methods. These antibodies may be generated according to methods known in the art, using prediction from hydrophobicity charts, as described in the “Anti-NOVX Antibodies” section below. For example the disclosed NOV9 protein have multiple hydrophilic regions, each of which can be used as an immunogen. In one embodiment, contemplatedNOV9 epitope is from about amino acids 50 to 70. In another embodiment, a NOV9 epitope is from about amino acids 105 to 120. In additional embodiments, NOV9 epitopes are from about amino acids 250 to 300, from about amino acids 350 to 370, from about 380 to 400, and from about amino acids 440 to 460. This novel protein also has value in development of powerful assay system for functional analysis of various human disorders, which will help in understanding of pathology of the disease and development of new drug targets for various disorders.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence SEQ ID NOS: 1, 3, 5, 7, 9, 11, 13, 15, 17, 19, 21, 23, and 25, or a complement of this aforementioned nucleotide sequence, can be isolated using standard molecular biology techniques and the sequence information provided herein. Using all or a portion of the nucleic acid sequence of SEQ ID NOS: 1, 3, 5, 7, 9, 11, 13, 15, 17, 19, 21, 23, and 25 as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides SEQ ID NOS: 1, 3, 5, 7, 9, 11, 13, 15, 17, 19, 21, 23, and 25, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS: 1, 3, 5, 7, 9, 11, 13, 15, 17, 19, 21, 23, and 25, or a portion of this nucleotide sequence (e.g., a fragment that can be used as a probe or primer or a fragment encoding a biologically-active portion of an NOVX polypeptide). A nucleic acid molecule that is complementary to the nucleotide sequence shown SEQ ID NOS: 1, 3, 5, 7, 9, 11, 13, 15, 17, 19, 21, 23, or 25 is one that is sufficiently complementary to the nucleotide sequence shown SEQ ID NOS: 1, 3, 5, 7, 9, 11, 13, 15, 17, 19, 21, 23, or 25 that it can hydrogen bond with little or no mismatches to the nucleotide sequence shown SEQ ID NOS: 1, 3, 5, 7, 9, 11, 13, 15, 17, 19, 21, 23, and 25, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NOS: 1, 3, 5, 7, 9, 11, 13, 15, 17, 19, 21, 23, and 25, as well as a polypeptide possessing NOVX biological activity. Various biological activities of the NOVX proteins are described below.

An NOVX polypeptide is encoded by the open reading frame (“ORF”) of an NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence SEQ ID NOS: 1, 3, 5, 7, 9, 11, 13, 15, 17, 19, 21, 23, or 25; or an anti-sense strand nucleotide sequence of SEQ ID NOS: 1, 3, 5, 7, 9, 11, 13, 15, 17, 19, 21, 23, or 25; or of a naturally occurring mutant of SEQ ID NOS: 1, 3, 5, 7, 9, 11, 13, 15, 17, 19, 21, 23, and 25.

Probes based on the human NOVX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an NOVX protein, such as by measuring a level of an NOVX-encoding nucleic acid in a sample of cells from a subject e.g., detecting NOVX mRNA levels or determining whether a genomic NOVX gene has been mutated or deleted.

“A polypeptide having a biologically-active portion of an NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion SEQ ID NOS: 1, 3, 5, 7, 9, 11, 13, 15, 17, 19, 21, 23, or 25, that encodes a polypeptide having an NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences shown in SEQ ID NOS: 1, 3, 5, 7, 9, 11, 13, 15, 17, 19, 21, 23, and 25 due to degeneracy of the genetic code and thus encode the same NOVX proteins as that encoded by the nucleotide sequences shown in SEQ ID NOS: 1, 3, 5, 7, 9, 11, 13, 15, 17, 19, 21, 23, and 25. In another embodiment, an isolated nucleic acid molecule of the invention has a nucleotide sequence encoding a protein having an amino acid sequence shown in SEQ ID NOS: 2, 4, 6, 8, 10, 12, 14, 16, 18, 20, 22, 24 or 26.

In addition to the human NOVX nucleotide sequences shown in SEQ ID NOS: 1, 3, 5, 7, 9, 11, 13, 15, 17, 19, 21, 23, and 25, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding an NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from the human SEQ ID NOS: 1, 3, 5, 7, 9, 11, 13, 15, 17, 19, 21, 23, and 25 are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS: 1, 3, 5, 7, 9, 11, 13, 15, 17, 19, 21, 23, and 25. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences SEQ ID NOS: 1, 3, 5, 7, 9, 11, 13, 15, 17, 19, 21, 23, and 25, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS: 1, 3, 5, 7, 9, 11, 13, 15, 17, 19, 21, 23, and 25, or fragments, analogs or derivatives thereof, under conditions of moderate stringency is provided. A non-limiting example of moderate stringency hybridization conditions are hybridization in 6×SSC, 5×Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, N.Y., and Kriegler, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences SEQ ID NOS: 1, 3, 5, 7, 9, 11, 13, 15, 17, 19, 21, 23, and 25, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, N.Y., and Kriegler, 1990, GENE TRANSFER AND EXPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences SEQ ID NOS: 1, 3, 5, 7, 9, 11, 13, 15, 17, 19, 21, 23, and 25, thereby leading to changes in the amino acid sequences of the encoded NOVX proteins, without altering the functional ability of said NOVX proteins. For example, nucleotide substitutions leading to amino acid substitutions at “non-essential” amino acid residues can be made in the sequence SEQ ID NOS: 2, 4, 6, 8, 10, 12, 14, 16, 18, 20, 22, 24 or 26. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NOS: 1, 3, 5, 7, 9, 11, 13, 15, 17, 19, 21, 23, and 25 yet retain biological activity. In one embodiment, the isolated nucleic acid molecule comprises a nucleotide sequence encoding a protein, wherein the protein comprises an amino acid sequence at least about 45% homologous to the amino acid sequences SEQ ID NOS: 2, 4, 6, 8, 10, 12, 14, 16, 18, 20, 22, 24 and 26. Preferably, the protein encoded by the nucleic acid molecule is at least about 60% homologous to SEQ ID NOS: 2, 4, 6, 8, 10, 12, 14, 16, 18, 20, 22, 24 and 26; more preferably at least about 70% homologous SEQ ID NOS: 2, 4, 6, 8, 10, 12, 14, 16, 18, 20, 22, 24 or 26; still more preferably at least about 80% homologous to SEQ ID NOS: 2, 4, 6, 8, 10, 12, 14, 16, 18, 20, 22, 24 or 26; even more preferably at least about 90% homologous to SEQ ID NOS: 2, 4, 6, 8, 10, 12, 14, 16, 18, 20, 22, 24 or 26; and most preferably at least about 95% homologous to SEQ ID NOS: 2, 4, 6, 8, 10, 12, 14, 16, 18, 20, 22, 24 or 26.

An isolated nucleic acid molecule encoding an NOVX protein homologous to the protein of SEQ ID NOS: 2, 4, 6, 8, 10, 12, 14, 16, 18, 20, 22, 24 or 26 can be created by introducing one or more nucleotide substitutions, additions or deletions into the nucleotide sequence of SEQ ID NOS: 1, 3, 5, 7, 9, 11, 13, 15, 17, 19, 21, 23, and 25, such that one or more amino acid substitutions, additions or deletions are introduced into the encoded protein.

Mutations can be introduced into SEQ ID NOS: 1, 3, 5, 7, 9, 11, 13, 15, 17, 19,21, 23, and 25 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g. lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of an NOVX coding sequence, such as by saturation mutagenesis, and the resultant mutants can be screened for NOVX biological activity to identify mutants that retain activity. Following mutagenesis SEQ ID NOS: 1, 3, 5, 7, 9, 11, 13, 15, 17, 19, 21, 23, and 25, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and an NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Antisense Nucleic Acids

Another aspect of the invention pertains to isolated antisense nucleic acid molecules that are hybridizable to or complementary to the nucleic acid molecule comprising the nucleotide sequence of SEQ ID NOS: 1, 3, 5, 7, 9, 11, 13, 15, 17, 19, 21, 23, and 25, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of an NOVX protein of SEQ ID NOS: 2, 4, 6, 8, 10, 12, 14, 16, 18, 20,22, 24 or 26, or antisense nucleic acids complementary to an NOVX nucleic acid sequence of SEQ ID NOS: 1, 3, 5, 7, 9, 11, 13, 15, 17, 19, 21, 23, and 25, are additionally provided.

In one embodiment, an antisense nucleic acid molecule is antisense to a “coding region” of the coding strand of a nucleotide sequence encoding an NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1 -methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding an NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An a-anomeric nucleic acid molecule forms specific double-stranded hybrids with complementary RNA in which, contrary to the usual P-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g. hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for an NOVX-encoding nucleic acid can be designed based upon the nucleotide sequence of an NOVX cDNA disclosed herein (i.e., SEQ ID NOS: 1, 3, 5, 7, 9, 11, 13, 15, 17, 19, 21, 23, and 25). For example, a derivative of a Tetrahymena L-19 IVS RNA can be constructed in which the nucleotide sequence of the active site is complementary to the nucleotide sequence to be cleaved in an NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al, 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g. Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in SEQ ID NOS: 2, 4, 6, 8, 10, 12, 14, 16, 18, 20, 22, 24 or 26. The invention also includes a mutant or variant protein any of whose residues may be changed from the corresponding residues shown in SEQ ID NOS: 2, 4, 6, 8, 10, 12, 14, 16, 18, 20, 22, 24 or 26 while still encoding a protein that maintains its NOVX activities and physiological functions, or a functional fragment thereof.

In general, an NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, an NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence shown in SEQ ID NOS: 2, 4, 6, 8, 10, 12, 14, 16, 18, 20, 22, 24 or 26) that include fewer amino acids than the full-length NOVX proteins, and exhibit at least one activity of an NOVX protein. Typically, biologically-active portions comprise a domain or motif with at least one activity of the NOVX protein. A biologically-active portion of an NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence shown SEQ ID NOS: 2, 4, 6, 8, 10, 12, 14, 16, 18, 20, 22, 24 or 26. In other embodiments, the NOVX protein is substantially homologous to SEQ ID NOS: 2, 4, 6, 8, 10, 12, 14, 16, 18, 20, 22, 24 or 26, and retains the functional activity of the protein of SEQ ID NOS: 2, 4, 6, 8, 10, 12, 14, 16, 18, 20, 22, 24 or 26, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence SEQ ID NOS: 2, 4, 6, 8, 10, 12, 14, 16, 18, 20, 22, 24 or 26, and retains the functional activity of the NOVX proteins of SEQ ID NOS: 2, 4, 6, 8, 10, 12, 14, 16, 18, 20, 22, 24 or 26.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J. Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS: 1, 3, 5, 7, 9, 11, 13, 15, 17, 19, 21, 23, and 25.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, an NOVX “chimeric protein” or “fusion protein” comprises an NOVX polypeptide operatively-linked to a non-NOVX polypeptide. An “NOVX polypeptide” refers to a polypeptide having an amino acid sequence corresponding to an NOVX protein SEQ ID NOS: 2, 4, 6, 8, 10, 12, 14, 16, 18, 20, 22, 24 or 26, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within an NOVX fusion protein the NOVX polypeptide can correspond to all or a portion of an NOVX protein. In one embodiment, an NOVX fusion protein comprises at least one biologically-active portion of an NOVX protein. In another embodiment, an NOVX fusion protein comprises at least two biologically-active portions of an NOVX protein. In yet another embodiment, an NOVX fusion protein comprises at least three biologically-active portions of an NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is an NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is an NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between an NOVX ligand and an NOVX protein on the surface of a cell, to thereby suppress NOVX-mediated signal transduction in vivo. The NOVX-immunoglobulin fusion proteins can be used to affect the bioavailability of an NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with an NOVX ligand.

An NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). An NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of an NOVX protein. In one embodiment, a library of coding sequence fragments can be generated by treating a double stranded PCR fragment of an NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with SI nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Also included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated NOVX-related protein of the invention may be intended to serve as an antigen, or a portion or fragment thereof, and additionally can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX-related protein that is located on the surface of the protein, e.g., a hydrophilic region. A hydrophobicity analysis of the human NOVX-related protein sequence will indicate which regions of a NOVX-related protein are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each of which is incorporated herein by reference in its entirety. Antibodies that are specific for one or more domains within an antigenic protein, or derivatives, fragments, analogs or homologs thereof, are also provided herein.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow and Lane, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa, Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosy1 transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., MONOCLONAL ANTIBODY PRODUCTION TECHNIQUES AND APPLICATIONS, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). Preferably, antibodies having a high degree of specificity and a high binding affinity for the target antigen are isolated.

After the desired hybridoma cells are identified, the clones can be subcloned by limiting dilution procedures and grown by standard methods. Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No.5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies relate to antibody molecules in which essentially the entire sequences of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., 1991 EMBO J., 10:3655-3659.

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor”(such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Anti-NOVX antibodies may be used in methods known within the art relating to the localization and/or quantitation of an NOVX protein (e.g., for use in measuring levels of the NOVX protein within appropriate physiological samples, for use in diagnostic methods, for use in imaging the protein, and the like). In a given embodiment, antibodies for NOVX proteins, or derivatives, fragments, analogs or homologs thereof, that contain the antibody derived binding domain, are utilized as pharmacologically-active compounds (hereinafter “Therapeutics”).

An anti-NOVX antibody (e.g., monoclonal antibody) can be used to isolate an NOVX polypeptide by standard techniques, such as affinity chromatography or immunoprecipitation. An anti-NOVX antibody can facilitate the purification of natural NOVX polypeptide from cells and of recombinantly-produced NOVX polypeptide expressed in host cells. Moreover, an anti-NOVX antibody can be used to detect NOVX protein (e.g., in a cellular lysate or cell supernatant) in order to evaluate the abundance and pattern of expression of the NOVX protein. Anti-NOVX antibodies can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

NOVX Recombinant Expression Vectors and Host Cells

Another aspect of the invention pertains to vectors, preferably expression vectors, containing a nucleic acid encoding an NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector.

Examples of vectors for expression in yeast Saccharomyces cerivisae include pYepSec1 (Baldari, et al., 1987. EMBO J. 6: 229-234), pMFa (Kuran and Herskowitz, 1982. Cell 30:

933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:

268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences SEQ ID NOS: 1, 3, 5, 7, 9, 11, 13, 15, 17, 19, 21, 23, and 25 can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. No. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of an NOVX gene into which a deletion, addition or substitution has been introduced to thereby alter, e.g., functionally disrupt, the NOVX gene. The NOVX gene can be a human gene (e.g., the cDNA of SEQ ID NOS: 1, 3, 5, 7, 9, 11, 13, 15, 17, 19, 21, 23, and 25), but more preferably, is a non-human homologue of a human NOVX gene. For example, a mouse homologue of human NOVX gene of SEQ ID NOS: 1, 3, 5, 7, 9, 11, 13, 15, 17, 19, 21, 23, and 25 can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O′Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, serotonin receptor, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in an NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g. peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of an NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37:1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to an NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I,³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with an NOVX target molecule. As used herein, a “target molecule” is a molecule with which an NOVX protein binds or interacts in nature, for example, a molecule on the surface of a cell which expresses an NOVX interacting protein, a molecule on the surface of a second cell, a molecule in the extracellular milieu, a molecule associated with the internal surface of a cell membrane or a cytoplasmic molecule. An NOVX target molecule can be a non-NOVX molecule or an NOVX protein or polypeptide of the invention. In one embodiment, an NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with an NOVX target molecule can be accomplished by one of the methods described above for determining direct binding. In one embodiment, determining the ability of the NOVX protein to bind to or interact with an NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising an NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting an NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with an NOVX protein, wherein determining the ability of the test compound to interact with an NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to an NOVX target molecule by one of the methods described above for determining direct binding. In an alternative embodiment, determining the ability of the test compound to modulate the activity of NOVX protein can be accomplished by determining the ability of the NOVX protein further modulate an NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with an NOVX protein, wherein determining the ability of the test compound to interact with an NOVX protein comprises determining the ability of the NOVX protein to preferentially bind to or modulate the activity of an NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesite®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also likely to be involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g. GAL4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences, SEQ ID NOS: 1, 3, 5, 7, 9, 11, 13, 15, 17, 19, 21, 23, and 25, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS: 1, 3, 5, 7, 9, 11, 13, 15, 17, 19, 21, 23, and 25 are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in an NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NOS: 1, 3, 5, 7, 9, 11, 13, 15, 17, 19, 21, 23, and 25, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in an NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an NOVX-protein, or the misexpression of the NOVX gene. For example, such genetic lesions can be detected by ascertaining the existence of at least one of: (i) a deletion of one or more nucleotides from an NOVX gene; (ii) an addition of one or more nucleotides to an NOVX gene; (iii) a substitution of one or more nucleotides of an NOVX gene, (iv) a chromosomal rearrangement of an NOVX gene; (v) an alteration in the level of a messenger RNA transcript of an NOVX gene, (vi) aberrant modification of an NOVX gene, such as of the methylation pattern of the genomic DNA, (vii) the presence of a non-wild-type splicing pattern of a messenger RNA transcript of an NOVX gene, (viii) a non-wild-type level of an NOVX protein, (ix) allelic loss of an NOVX gene, and (x) inappropriate post-translational modification of an NOVX protein. As described herein, there are a large number of assay techniques known in the art which can be used for detecting lesions in an NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to an NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on an NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g. Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g. Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, and other diseases, disorders and conditions of the like.

These methods of treatment will be discussed more fully, below.

Disease and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof, (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, an NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of an NOVX protein, a peptide, an NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of an NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering an NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease, Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be farther described in the following examples, which do not limit the scope of the invention described in the claims.

### EXAMPLES

### Example 1

### Identification of NOVX clones

The novel NOVX target sequences identified in the present invention were subjected to the exon linking process to confirm the sequence. PCR primers were designed by starting at the most upstream sequence available, for the forward primer, and at the most downstream sequence available for the reverse primer. Table 10A shows the sequences of the PCR primers used for obtaining different clones. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. Table 17B shows a list of these bacterial clones. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

TABLE 10A
PCR Primers for Exon Linking

| | | SEQ | | SEQ |
|---|---|---|---|---|
| NOVX | | ID | | ID |
| Clone | Primer 1 (5′-3′) | NO | Primer 2 (5′-3′) | NO |
| NOV1 | CCCTGTGGGGCCGGCTGCATCT | 42 | AGCTCAGGTCGGGTTCTCGTAGCTGGTGAA | 43 |
| NOV2 | AAGCTGCTCATCTTCAACACATACCAG | 44 | GCCTGCACGTCCCTGTCAC | 45 |
| NOV6B | ATGGTCACAGCCATGAATGTCTCACAT | 46 | CTTCACTGGCTCTTGGTCTTGGCTTT | 47 |

Physical clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

TABLE 10B

Physical Clones for PCR products

| | |
|---|---|
| NOVX Clone | Bacterial Clone |

| | |
|---|---|
| NOV2 | AC024920 |
| NOV6 | Ba242b12 |
| NOV9 | GM358d14, AL353658, AI373274 |

### Example 2

### Quantitative expression analysis of clones in various cells and tissues

The quantitative expression of various clones was assessed using microtiter plates containing RNA samples from a variety of normal and pathology-derived cells, cell lines and tissues using real time quantitative PCR (RTQ PCR). RTQ PCR was performed on a Perkin-Elmer Biosystems ABI PRISM® 7700 Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), AI_comprehensive_panel (containing normal tissue and samples from autoinflammatory diseases), Panel CNSD.01 (containing samples from normal and diseased brains) and CNS_neurodegeneration_panel (containing samples from normal and diseased brains).

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1×TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/μl RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

Panel 1

In the results for Panel 1, the following abbreviations are used:

ca.=carcinoma,

*=established from metastasis,

met=metastasis,

s cell var=small cell variant,

non-s=non-sm=non-small,

squam=squamous,

p1.eff=p1 effusion=pleural effusion,

glio=glioma,

astro=astrocytoma, and

neuro=neuroblastoma.

Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene,La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μ/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10^{31 5} M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) pateins, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: Hippocampus, Temporal cortex (Broddmann Area 21), Somatosensory cortex (Broddmann area 7), and Occipital cortex (Brodmann area 17). These regions were chosen to encompass all levels of neurodegeneration in AD. The hippocampus is a region of early and severe neuronal loss in AD; the temporal cortex is known to show neurodegeneration in AD after the hippocampus; the somatosensory cortex shows moderate neuronal death in the late stages of the disease; the occipital cortex is spared in AD and therefore acts as a “control” region within AD patients. Not all brain regions are represented in all cases.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

NOV2c

Expression of the gene NOV2c was assessed using the primer-probe sets Ag3073 described in Table 12. Results from RTQ-PCR runs are shown in Tables 13, 14, 15, 16, and 17.

TABLE 12

Probe Name Ag3O73

| | | | | |
|---|---|---|---|---|
| | | | | Start |
| Primers | Sequences | TM | Length | Position |

| | | | | |
|---|---|---|---|---|
| Forward | 5′-CTGCAGTCCCAGATCTCAGA-3′ (SEQ ID NO: 48) | 59.1 | 20 | 706 |
| Probe | FAM-5′-GTCTGTGGTGCTGTCCATGGACAAC-3′- (SEQ ID NO: 49) | 69.2 | 25 | 729 |
| | TAMRA | | | |
| Reverse | 5′-TACTGTGCCTTGACCTCAGC-3′ (SEQ ID NO: 50) | 59 | 20 | 784 |

TABLE 13
Panel 1.3D

| | Relative | | Relative |
|---|---|---|---|
| | Expression (%) | | Expression (%) |
| | 1.3dx4tm4877 | | 1.3dx4tm4877 |
| Tissue Name | f_ag3073_bl | Tissue Name | f_ag3073_bl |
| Liver adenocarcinoma | 89.8 | Kidney (fetal) | 5.7 |
| Pancreas | 1.0 | Renal ca. 786-0 | 0.9 |
| Pancreatic ca. | 49.2 | Renal ca. A498 | 2.0 |
| CAPAN 2 | | | |
| Adrenal gland | 1.2 | Renal ca. RXF 393 | 0.5 |
| Thyroid | 55.9 | Renal ca. ACHN | 2.3 |
| Salivary gland | 9.1 | Renal ca. UO-31 | 3.5 |
| Pituitary gland | 0.9 | Renal ca. TK-10 | 1.6 |
| Brain (fetal) | 0.1 | Liver | 0.4 |
| Brain (whole) | 0.2 | Liver (fetal) | 1.1 |
| Brain (amygdala) | 0.2 | Liver ca. (hepatoblast) | 1.9 |
| | | HepG2 | |
| Brain (cerebellum) | 0.6 | Lung | 31.7 |
| Brain (hippocampus) | 0.4 | Lung (fetal) | 13.7 |
| Brain (substantia | 0.1 | Lung ca. (small cell) | 24.1 |
| nigra) | | LX-1 | |
| Brain (thalamus) | 0.3 | Lung ca. (small cell) | 1.1 |
| | | NCI-H69 | |
| Cerebral Cortex | 2.4 | Lung ca. (s. cell var.) | 1.8 |
| | | SHP-77 | |
| Spinal cord | 1.1 | Lung ca. (large cell) | 1.6 |
| | | NCI-H460 | |
| CNS ca. (glio/astro) | 1.1 | Lung ca. (non-sm. cell) | 34.8 |
| U87-MG | | A549 | |
| CNS ca. (glio/astro) | 0.5 | Lung ca. (non-s.cell) | 1.1 |
| U-118-MG | | NCI-H23 | |
| CNS ca. (astro) | 20.9 | Lung ca (non-s.cell) | 13.0 |
| SW1783 | | HOP-62 | |
| CNS ca.* (neuro; met) | 0.3 | Lung ca. (non-s.cl) | 0.4 |
| SK-N-AS | | NCI-H522 | |
| CNS ca. (astro) | 0.9 | Lung ca. (squam.) | 28.2 |
| SF-539 | | SW 900 | |
| CNS ca. (astro) | 17.5 | Lung ca. (squam.) | 0.4 |
| SNB-75 | | NCI-H596 | |
| CNS ca. (glio) | 0.8 | Mammary gland | 19.9 |
| SNB-19 | | | |
| CNS ca. (glio) | 1.2 | Breast ca.* (pl. effusion) | 45.1 |
| U251 | | MCF-7 | |
| CNS ca. (glio) | 0.4 | Breast ca.* (pl.ef) | 32.9 |
| SF-295 | | MDA-MB-231 | |
| Heart (fetal) | 5.0 | Breast ca.* (pl. effusion) | 8.2 |
| | | T47D | |
| Heart | 0.7 | Breast ca. BT-549 | 5.9 |
| Fetal Skeletal | 2.2 | Breast ca. MDA-N | 0.5 |
| Skeletal muscle | 0.2 | Ovary | 4.6 |
| Bone marrow | 0.5 | Ovarian ca. OVCAR-3 | 44.2 |
| Thymus | 4.4 | Ovarian ca. OVCAR-4 | 65.0 |
| Spleen | 0.6 | Ovarian ca. OVCAR-5 | 22.2 |
| Lymph node | 0.7 | Ovarian ca. OVCAR-8 | 15.5 |
| Colorectal | 3.4 | Ovarian ca. IGROV-1 | 1.0 |
| Stomach | 0.9 | Ovarian ca.* (ascites) | 39.0 |
| | | SK-OV-3 | |
| Small intestine | 1.8 | Uterus | 0.3 |
| Colon ca. SW480 | 10.5 | Placenta | 42.1 |
| Colon ca.* (SW480 | 5.3 | Prostate | 4.3 |
| met)SW620 | | | |
| Colon ca. HT29 | 14.1 | Prostate ca.* (bone | 4.2 |
| | | met)PC-3 | |
| Colon ca. HCT-116 | 5.1 | Testis | 0.4 |
| Colon ca. CaCo-2 | 12.1 | Melanoma Hs688(A).T | 0.6 |
| 83219 CC Well to | 22.8 | Melanoma* (met) Hs688 | 5.7 |
| Mod Diff (ODO3866) | | (B).T | |
| Colon ca. HCC-2998 | 2.3 | Melanoma UACC-62 | 0.2 |
| Gastric ca.* (liver met) | 100.0 | Melanoma M14 | 0.2 |
| NCI-N87 | | | |
| Bladder | 23.7 | Melanoma LOX IMVI | 0.1 |
| Trachea | 41.0 | Melanoma* (met) | 0.3 |
| | | SK-MEL-5 | |
| Kidney | 8.7 | Adipose | 3.1 |

TABLE 14
Panel 2D

| | Relative | | Relative |
|---|---|---|---|
| | Expression (%) | | Expression (%) |
| | 2dx4tm4819f_{—} | | 2dx4tm4819f_{—} |
| Tissue Name | ag3073_a2 | Tissue Name | ag3073_a2 |
| Normal Colon | 9.6 | Kidney NAT Clontech | 7.4 |
| GENPAK 061003 | | 8120608 | |
| 83219 CC Well to | 9.0 | Kidney Cancer | 2.7 |
| Mod Duff (ODO3866) | | Clontech 8120613 | |
| 83220 CC NAT | 3.7 | Kidney NAT Clontech | 5.6 |
| (ODO3866) | | 8120614 | |
| 83221 CC Gr.2 | 2.2 | Kidney Cancer | 1.0 |
| rectosigmoid | | Clontech 9010320 | |
| (ODO3868) | | | |
| 83222 CC NAT | 0.2 | Kidney NAT Clontech | 7.6 |
| (ODO3868) | | 9010321 | |
| 83235 CC Mod Duff | 2.5 | Normal Uterus | 0.2 |
| (ODO3920) | | GENPAK 061018 | |
| 83236 CC NAT | 2.4 | Uterus Cancer | 5.0 |
| (ODO3920) | | GENPAK 064011 | |
| 83237 CC Gr.2 ascend | 6.2 | Normal Thyroid | 31.5 |
| colon (ODO3921) | | Clontech A+ | |
| | | 6570-1 | |
| 83238 CC NAT | 1.5 | Thyroid Cancer | 100.0 |
| (ODO3921) | | GENPAK 064010 | |
| 83241 CC from Partial | 10.8 | Thyroid Cancer | 31.9 |
| Hepatectomy | | INVITROGEN A302152 | |
| (ODO4309) | | | |
| 83242 Liver NAT | 1.3 | Thyroid NAT | 61.9 |
| (ODO4309) | | INVITROGEN A302153 | |
| 87472 Colon mets to | 5.8 | Normal Breast | 30.4 |
| lung (OD04451-01) | | GENPAK 061019 | |
| 87473 Lung NAT | 20.4 | 84877 Breast Cancer | 6.6 |
| (OD04451-02) | | (OD04566) | |
| Normal Prostate | 16.4 | 85975 Breast Cancer | 15.2 |
| Clontech A+ 6546-1 | | (OD04590-01) | |
| 84140 Prostate Cancer | 3.5 | 85976 Breast Cancer | 27.2 |
| (OD04410) | | Mets (OD04590-03) | |
| 84141 Prostate NAT | 1.4 | 87070 Breast Cancer | 22.0 |
| (OD04410) | | Metastasis | |
| | | (OD04655-05) | |
| 87073 Prostate Cancer | 8.5 | GENPAK Breast Cancer | 24.2 |
| (OD04720-01) | | 064006 | |
| 87074 Prostate NAT | 8.8 | Breast Cancer Res. | 48.5 |
| (OD04720-02) | | Gen. 1024 | |
| Normal Lung | 12.2 | Breast Cancer Clontech | 19.3 |
| GENPAK 061010 | | 9100266 | |
| 83239 Lung Met to | 3.3 | Breast NAT Clontech | 13.7 |
| Muscle (ODO4286) | | 9100265 | |
| 83240 Muscle NAT | 0.2 | Breast Cancer | 82.2 |
| (ODO4286) | | INVITROGEN | |
| | | A209073 | |
| 84136 Lung Malignant | 45.8 | Breast NAT | 13.2 |
| Cancer (OD03126) | | INVITROGEN | |
| | | A2090734 | |
| 84137 Lung NAT | 18.7 | Normal Liver | 0.6 |
| (OD03126) | | GENPAK 061009 | |
| 84871 Lung Cancer | 16.7 | Liver Cancer | 0.6 |
| (OD04404) | | GENPAK 064003 | |
| 84872 Lung NAT | 21.6 | Liver Cancer Research | 1.2 |
| (OD04404) | | Genetics RNA 1025 | |
| 84875 Lung Cancer | 3.6 | Liver Cancer Research | 4.0 |
| (OD04565) | | Genetics RNA 1026 | |
| 84876 Lung NAT | 14.0 | Paired Liver Cancer | 1.9 |
| (OD04565) | | Tissue Research Genetics | |
| | | RNA 6004-T | |
| 85950 Lung Cancer | 21.1 | Paired Liver Tissue | 1.0 |
| (OD04237-01) | | Research Genetics | |
| | | RNA 6004-N | |
| 85970 Lung NAT | 31.0 | Paired Liver Cancer | 3.3 |
| (OD04237-02) | | Tissue Research Genetics | |
| | | RNA 6005-T | |
| 83255 Ocular Mel Met | 0.2 | Paired Liver Tissue | 0.7 |
| to Liver (ODO4310) | | Research Genetics | |
| | | RNA 6005-N | |
| 83256 Liver NAT | 3.5 | Normal Bladder | 24.1 |
| (ODO4310) | | GENPAK 061001 | |
| 84139 Melanoma Mets | 1.4 | Bladder Cancer Research | 3.5 |
| to Lung (OD04321) | | Genetics RNA 1023 | |
| 84138 Lung NAT | 41.5 | Bladder Cancer | 2.6 |
| (OD04321) | | INVITROGEN A302173 | |
| Normal Kidney | 9.0 | 87071 Bladder Cancer | 64.8 |
| GENPAK 061008 | | (OD04718-01) | |
| 83786 Kidney Ca, | 37.2 | 87072 Bladder Normal | 0.6 |
| Nuclear grade 2 | | Adjacent (OD04718-03) | |
| (OD04338) | | | |
| 83787 Kidney NAT | 10.0 | Normal Ovary Res. Gen. | 0.6 |
| (OD04338) | | | |
| 83788 Kidney Ca | 14.3 | Ovanan Cancer | 40.9 |
| Nuclear grade 1/2 | | GENPAK 064008 | |
| (OD04339) | | | |
| 83789 Kidney NAT | 6.6 | 87492 Ovary Cancer | 50.8 |
| (OD04339) | | (OD04768-07) | |
| 83790 Kidney Ca, | 2.3 | 87493 Ovary NAT | 1.6 |
| Clear cell type | | (OD04768-08) | |
| (OD04340) | | | |
| 83791 Kidney NAT | 6.0 | Normal Stomach | 2.6 |
| (OD04340) | | GENPAK 061017 | |
| 83792 Kidney Ca, | 0.5 | Gastric Cancer | 1.1 |
| Nuclear grade 3 | | Clontech 9060358 | |
| (OD04348) | | | |
| 83793 Kidney NAT | 8.2 | NAT Stomach | 2.9 |
| (OD04348) | | Clontech 9060359 | |
| 87474 Kidney Cancer | 1.1 | Gastric Cancer | 3.6 |
| (OD04622-01) | | Clontech 9060395 | |
| 87475 Kidney NAT | 4.4 | NAT Stomach | 3.8 |
| (OD04622-03) | | Clontech 9060394 | |
| 85973 Kidney Cancer | 46.0 | Gastric Cancer | 14.2 |
| (OD04450-01) | | Clontech 9060397 | |
| 85974 Kidney NAT | 11.1 | NAT Stomach | 4.9 |
| (OD04450-03) | | Clontech 9060396 | |
| Kidney Cancer | 1.6 | Gastric Cancer | 7.5 |
| Clontech 8120607 | | GENPAK 064005 | |

TABLE 15
Panel 2.2

| | Relative | | Relative |
|---|---|---|---|
| | Expression (%) | | Expression (%) |
| | 2.2x4tm6406f_{—} | | 2.2x4tm6406f_{—} |
| Tissue Name | ag3073_bl | Tissue Name | ag3073_bl |
| Normal Colon | 3.6 | 83793 Kidney NAT | 36.0 |
| GENPAK 061003 | | (OD04348) | |
| 97759 Colon cancer | 21.9 | 98938 Kidney malignant | 100.0 |
| (OD06064) | | cancer (OD06204B) | |
| 97760 Colon cancer | 1.1 | 98939 Kidney normal | 8.0 |
| NAT (OD06064) | | adjacent tissue | |
| | | (OD06204E) | |
| 97778 Colon cancer | 5.9 | 85973 Kidney Cancer | 83.8 |
| (OD06159) | | (OD04450-01) | |
| 97779 Colon cancer | 4.1 | 85974 Kidney NAT | 8.5 |
| NAT (OD06159) | | (OD04450-03) | |
| 98861 Colon cancer | 1.0 | Kidney Cancer Clontech | 1.0 |
| (OD06297-04) | | 8120613 | |
| 98862 Colon cancer | 4.6 | Kidney NAT Clontech | 6.7 |
| NAT (OD06297-015) | | 8120614 | |
| 83237 CC Gr.2 ascend | 1.6 | Kidney Cancer Clontech | 0.8 |
| colon (ODO3921) | | 9010320 | |
| 83238 CC NAT | 1.5 | Kidney NAT Clontech | 3.8 |
| (ODO3921) | | 9010321 | |
| 97766 Colon cancer | 3.2 | Kidney Cancer Clontech | 3.8 |
| metastasis (OD06104) | | 8120607 | |
| 97767 Lung NAT | 2.2 | Kidney NAT Clontech | 7.8 |
| (OD06104) | | 8120608 | |
| 87472 Colon mets to | 13.6 | Normal Uterus | 0.6 |
| lung (OD04451-01) | | GENPAK 061018 | |
| 87473 Lung NAT | 30.0 | Uterus Cancer | 3.1 |
| (OD04451-02) | | GENPAK 064011 | |
| Normal Prostate | 3.5 | Normal Thyroid | 21.9 |
| Clontech A+ | | Clontech A+ | |
| 6546-1 (8090438) | | 6570-1 (7080817) | |
| 84140 Prostate | 0.8 | Thyroid Cancer | 57.1 |
| Cancer (OD04410) | | GENPAK 064010 | |
| 84141 Prostate NAT | 0.7 | Thyroid Cancer | 51.3 |
| (OD04410) | | INVITROGEN A302152 | |
| Normal Ovary Res. | 1.9 | Thyroid NAT | 20.5 |
| Gen. | | INVITROGEN A302153 | |
| 98863 Ovarian cancer | 12.7 | Normal Breast GENPAK | 23.2 |
| (OD06283-03) | | 061019 | |
| 98865 Ovarian cancer | 1.5 | 84877 Breast Cancer | 2.6 |
| NAT/fallopian tube | | (OD04566) | |
| (OD06283-07) | | | |
| Ovarian Cancer | 15.3 | Breast Cancer Res. | 51.7 |
| GENPAK 064008 | | Gen. 1024 | |
| 97773 Ovarian cancer | 4.9 | 85975 Breast Cancer | 29.2 |
| (OD06145) | | (OD04590-01) | |
| 97775 Ovarian cancer | 6.8 | 85976 Breast Cancer | 18.3 |
| NAT (OD06145) | | Mets (OD04590-03) | |
| 98853 Ovarian cancer | 15.7 | 87070 Breast Cancer | 22.3 |
| (OD06455-03) | | Metastasis | |
| | | (OD04655-05) | |
| 98854 Ovarian NAT | 0.2 | GENPAK Breast Cancer | 23.8 |
| (OD06455-07) | | 064006 | |
| Fallopian tube | | | |
| Normal Lung | 5.5 | Breast Cancer Clontech | 7.0 |
| GENPAK 061010 | | 9100266 | |
| 92337 Invasive poor | 31.6 | Breast NAT Clontech | 7.9 |
| diff. lung adeno | | 9100265 | |
| (OD04945-01) | | | |
| 92338 Lung NAT | 15.4 | Breast Cancer | 21.2 |
| (OD04945-03) | | INVITROGEN | |
| | | A209073 | |
| 84136 Lung Malignant | 27.3 | Breast NAT | 15.1 |
| Cancer (OD03126) | | INVITROGEN | |
| | | A2090734 | |
| 84137 Lung NAT | 6.6 | 97763 Breast cancer | 83.3 |
| (OD03126) | | (OD06083) | |
| 90372 Lung Cancer | 13.3 | 97764 Breast cancer | 36.6 |
| (OD05014A) | | node metastasis | |
| | | (OD06083) | |
| 90373 Lung NAT | 13.9 | Normal Liver GENPAK | 1.8 |
| (OD05014B) | | 061009 | |
| 97761 Lung cancer | 5.7 | Liver Cancer Research | 2.9 |
| (OD06081) | | Genetics RNA 1026 | |
| 97762 Lung cancer | 13.2 | Liver Cancer Research | 5.3 |
| NAT (OD06081) | | Genetics RNA 1025 | |
| 85950 Lung Cancer | 9.3 | Paired Liver Cancer | 3.9 |
| (OD04237-01) | | Tissue Research Genetics | |
| | | RNA 6004-T | |
| 85970 Lung NAT | 56.3 | Paired Liver Tissue | 1.4 |
| (OD04237-02) | | Research Genetics | |
| | | RNA 6004-N | |
| 83255 Ocular Mel Met | 0.2 | Paired Liver Cancer | 9.1 |
| to Liver (OD04310) | | Tissue Research Genetics | |
| | | RNA 6005-T | |
| 83256 Liver NAT | 4.2 | Paired Liver Tissue | 6.6 |
| (OD04310) | | Research Genetics | |
| | | RNA 6005-N | |
| 84139 Melanoma Mets | 1.8 | Liver Cancer | 3.5 |
| to Lung (OD04321) | | GENPAK 064003 | |
| 84138 Lung NAT | 24.9 | Normal Bladder | 15.3 |
| (OD04321) | | GENPAK 061001 | |
| Normal Kidney | 3.6 | Bladder Cancer | 5.6 |
| GENPAK 061008 | | Research Genetics | |
| | | RNA 1023 | |
| 83786 Kidney Ca, | 20.3 | Bladder Cancer | 4.7 |
| Nuclear grade 2 | | INVITROGEN | |
| (OD04338) | | A302173 | |
| 83787 Kidney NAT | 14.5 | Normal Stomach | 7.5 |
| (OD04338) | | GENPAK 061017 | |
| 83788 Kidney Ca | 29.8 | Gastric Cancer | 3.0 |
| Nuclear grade 1/2 | | Clontech 9060397 | |
| (OD04339) | | | |
| 83789 Kidney NAT | 6.3 | NAT Stomach | 7.2 |
| (OD04339) | | Clontech 9060396 | |
| 83790 Kidney Ca, | 1.6 | Gastric Cancer | 3.2 |
| Clear cell type | | Clontech 9060395 | |
| (OD04340) | | | |
| 83791 Kidney NAT | 6.7 | NAT Stomach | 6.1 |
| (OD04340) | | Clontech 9060394 | |
| 83792 Kidney Ca, | 0.5 | Gastric Cancer | 5.0 |
| Nuclear grade 3 | | GENPAK 064005 | |
| (OD04348) | | | |

TABLE 16
Panel 3D

| | Relative | | Relative |
|---|---|---|---|
| | Expression (%) | | Expression (%) |
| | 3dtm5232f_{—} | | 3dtm5232f_{—} |
| Tissue Name | ag3073 | Tissue Name | ag3073 |
| 94905_Daoy_Medulloblastoma/ | 0.0 | 94954_Ca Ski_Cervical | 100.0 |
| Cerebellum_sscDNA | | epidermoid carcinoma | |
| | | (metastasis)_sscDNA | |
| 94906_TE671_Medulloblastom/ | 0.0 | 94955_ES-2_Ovarian clear cell | 6.4 |
| Cerebellum_sscDNA | | carcinoma_sscDNA | |
| 94907_D283 | 0.3 | 94957_Ramos/6h stim_{—} | 0.0 |
| Med_Medulloblastoma/Cerebell | | Stimulated with | |
| um_sscDNA | | PMA/ionomycin 6h_sscDNA | |
| 94908_PFSK-1_Primitive | 0.0 | 94958_Ramos/14h stim_{—} | 0.0 |
| Neuroectodermal/Cerebellum_{—sscDNA} | | Stimulated with | |
| | | PMA/ionomycin 14h_sscDNA | |
| 94909_XF-498_CNS_sscDNA | 0.0 | 94962_MEG-01_Chronic | 0.1 |
| | | myelogenous leukemia | |
| | | (megokaryoblast)_sscDNA | |
| 94910_SNB- | 0.2 | 94963_Raji_Burkitt's | 0.1 |
| 78_CNS/glioma_sscDNA | | lymphoma_sscDNA | |
| 94911_SF- | 0.0 | 94964_Daudi_Burkitt's | 0.2 |
| 268_CNS/glioblastoma_sscDNA | | lymphoma_sscDNA | |
| 94912_T98G_Glio- | 0.0 | 94965_U266_B-cell | 0.1 |
| blastoma_sscDNA | | plasmacytoma/myeloma_sscDNA | |
| 96776_SK-N- | 0.0 | 94968_CA46_Burkitt's | 0.0 |
| SH_Neuroblastoma | | lymphoma_sscDNA | |
| (metastasis)_sscDNA | | | |
| 94913_SF- | 0.0 | 94970_RL_non-Hodgkin's B- | 0.0 |
| 295_CNS/glio- | | cell lymphoma_sscDNA | |
| blastoma_sscDNA | | | |
| 94914_Cerebellum_sscDNA | 0.2 | 94972_JM1_pre-B-cell | 0.1 |
| | | lymphoma/leukemia_sscDNA | |
| 96777_Cerebellum_sscDNA | 0.2 | 94973_Jurkat_T cell | 0.1 |
| | | leukemia_sscDNA | |
| 94916_NCI- | 93.3 | 94974_TF- | 0.1 |
| H292_Mucoepidermoid lung | | 1_Erythroleukemia_{—sscDNA} | |
| carcinoma_sscDNA | | | |
| 94917_DMS-114_Small | 0.0 | 94975_HUT 78_T-cell | 0.1 |
| cell lung cancer_sscDNA | | lymphoma_sscDNA | |
| 94918_DMS-79_Small cell | 9.0 | 94977_U937_Histiocytic | 0.1 |
| lung cancer/neuroendocrine_sscDNA | | lymphoma_sscDNA | |
| 94919_NCI-H146_Small cell | 0.3 | 94980_KU-812_Myelogenous | 0.1 |
| lung cancer/neuroendocrine_sscDNA | | leukemia_sscDNA | |
| 94920_NCI-H526_Small cell | 0.2 | 94981_769-P_Clear | 0.3 |
| lung cancer/neuroendocrine_sscDNA | | cell renal carcinoma_sscDNA | |
| 94921_NCI-N417_Small cell | 0.0 | 94983_Caki-2_Clear | 0.5 |
| lung cancer/neuroendocrine_sscDNA | | cell renal carcinoma_sscDNA | |
| 94923_NCI-H82_Small cell | 0.1 | 94984_SW 839_Clear | 0.3 |
| lung cancer/neuroendocrine_sscDNA | | cell renal carcinoma_sscDNA | |
| 94924_NCI-H157_Squamous | 0.1 | 94986_G401 Wilms' | 0.5 |
| cell lung cancer | | tumor_sscDNA | |
| (metastasis)_sscDNA | | | |
| 94925_NCI-H1155_Large cell | 0.4 | 94987_Hs766T_Pancreatic | 24.8 |
| lung cancer/neuroendocrine_sscDNA | | carcinoma (LN metastasis)_sscDNA | |
| 94926_NCI-H1299_Large cell | 0.2 | 94988_CAPAN-1_Pancreatic | 38.4 |
| lung cancer/neuroendocrine_sscDNA | | adenocarcinoma (liver | |
| | | metastasis)_sscDNA | |
| 94927_NCI-H727_Lung | 13.7 | 94989_SU86.86_Pancreatic | 61.1 |
| carcinoid_sscDNA | | carcinoma (liver | |
| | | metastasis)_sscDNA | |
| 94928_NCI-UMC-11_Lung | 11.3 | 94990_BxPC-3_Pancreatic | 33.0 |
| carcinoid_sscDNA | | adenocarcinoma_sscDNA | |
| 94929_LX-1_Small cell lung | 16.2 | 94991_HPAC_Pancreatic | 63.3 |
| cancer_sscDNA | | adenocarcinoma_sscDNA | |
| 94930_Colo-205_Colon | 3.0 | 94992_MIA PaCa-2_Pancreatic | 0.9 |
| cancer_sscDNA | | carcinoma_sscDNA | |
| 94931_KM12_Colon | 7.9 | 94993_CFPAC-1_Pancreatic | 46.0 |
| cancer_sscDNA | | ductal | |
| | | adenocarcinoma_sscDNA | |
| 94932_KM20L2_Colon | 2.4 | 94994_PANC-1_Pancreatic | 5.6 |
| cancer_sscDNA | | epithelioid ductal | |
| | | carcinoma_sscDNA | |
| 94933_NCI-H716_Colon | 0.5 | 94996_T24_Bladder carcinma | 24.3 |
| cancer_sscDNA | | (transitional cell)_sscDNA | |
| 94935_SW-48_Colon | 0.7 | 94997_5637_Bladder | 29.3 |
| adenocarcinoma_sscDNA | | carcinoma_sscDNA | |
| 94936_SW1l16_Colon | 0.5 | 94998_HT-1197_Bladder | 51.4 |
| adenocarcinoma_sscDNA | | carcinoma_sscDNA | |
| 94937_LS 174T_Colon | 3.6 | 94999_UM-UC-3_Bladder | 0.0 |
| adenocarcinoma_sscDNA | | carcinma (transitional | |
| | | cell)_sscDNA | |
| 94938_SW-948_Colon | 0.3 | 95000_A204_Rhabdomyo- | 0.0 |
| adenocarcinoma_sscDNA | | sarcoma_sscDNA | |
| 94939_SW-480_Colon | 0.4 | 95001_HT- | 0.2 |
| adenocarcinoma_sscDNA | | 1080_Fibrosarcoma_sscDNA | |
| 94940_NCI-SNU-5_Gastric | 4.9 | 95002_MG-63_Osteosarcoma | 0.0 |
| carcinoma_sscDNA | | (bone)_sscDNA | |
| 94941_KATO III_Gastric | 26.6 | 95003_SK-LMS- | 4.6 |
| carcinoma_sscDNA | | 1_Lelomyosarcoma | |
| | | (vulva)_sscDNA | |
| 94943_NCI-SNU-16_Gastric | 0.3 | 95004_SJRH30_Rhabdomyosar | 0.0 |
| carcinoma_sscDNA | | coma (met to bone | |
| | | marrow)_sscDNA | |
| 94944_NCI-SNU-l_Gastric | 14.2 | 95005_A431_Epidermoid | 0.8 |
| carcinoma_sscDNA | | carcinoma_sscDNA | |
| 94946_RF-1 Gastric | 0.0 | 95007_WM266- | 0.0 |
| adenocarcinoma_sscDNA | | 4_Melanoma_sscDNA | |
| 94947_RF-48_Gastric | 0.0 | 95010_DU 145_Prostate | 0.1 |
| adenocarcinoma_sscDNA | | carcinoma (brain | |
| | | metastasis)_sscDNA | |
| 96778_MKN-45_Gastric | 8.7 | 95012_MDA-MB-468_Breast | 15.4 |
| carcinoma_sscDNA | | adenocarcinoma_sscDNA | |
| 94949_NCI-N87_Gastric | 19.8 | 95013_SCC-4_Squamous cell | 0.4 |
| carcinoma_sscDNA | | carcinoma of tongue_sscDNA | |
| 94951_OVCAR-5_Ovarian | 8.2 | 95014_SCC-9_Squamous cell | 0.0 |
| carcinoma_sscDNA | | carcinoma of tongue_sscDNA | |
| 94952_RL95-2_Uterine | 12.2 | 95015_SCC-15_Squamous cell | 0.0 |
| carcinoma_sscDNA | | carcinoma of tongue_sscDNA | |
| 94953_HelaS3_Cervical | 8.6 | 95017_CAL 27_Squamous cell | 0.7 |
| adenocarcinoma_sscDNA | | carcinoma of tongue_sscDNA | |

TABLE 17
Panel 4D

| | Relative | | Relative |
|---|---|---|---|
| | Expression (%) | | Expression (%) |
| | 4dtm4705f_ag | | 4dtm4705f_ag |
| Tissue Name | 3703 | Tissue Name | 3703 |
| 93768_Secondary Th1_anti- | 0.3 | 93100_HUVEC | 0.2 |
| CD28/anti-CD3 | | (Endothelial)_IL-lb | |
| 93769_Secondary Th2_anti- | 0.5 | 93779_HUVEC | 2.0 |
| CD28/anti-CD3 | | (Endothelial)_IFN gamma | |
| 93770_Secondary Tr1_anti- | 0.6 | 93102_HUVEC | 1.0 |
| CD28/anti-CD3 | | (Endothelial)TNF alpha + IFN | |
| | | gamma | |
| 93573_Secondary Th1_resting | 0.2 | 93101_HUVEC | 3.1 |
| day 4-6 in IL-2 | | (Endothelial)_TNF alpha + IL4 | |
| 93572_Secondary Th2_resting | 0.3 | 93781_HUVEC | 2.0 |
| day 4-6 in IL-2 | | (Endothelial)_IL-11 | |
| 93571_Secondary Tr1_resting | 17.3 | 93583_Lung Microvascular | 25.5 |
| day 4-6 in IL-2 | | Endothelial Cells_none | |
| 93568_primary Th1_anti- | 0.6 | 93584_Lung Microvascular | 20.6 |
| CD28/anti-CD3 | | Endothelial Cells_TNFa (4 | |
| | | ng/ml) and IL1b (1 ng/ml) | |
| 93569_primary Th2_anti- | 0.4 | 92662_Microvascular Dermal | 55.1 |
| CD28/anti-CD3 | | endothelium_none | |
| 93570_primary Tr1_anti- | 0.7 | 92663_Microsvasular Dermal | 26.1 |
| CD28/anti-CD3 | | endothelium_TNFa (4 ng/ml) | |
| | | and IL1b (1 ng/ml) | |
| 93565_primary Th1_resting dy | 1.4 | 93773_Bronchial | 13.3 |
| 4-6 in IL-2 | | epithelium_TNFa (4 ng/ml) and | |
| | | IL1b (1 ng/ml)** | |
| 93566_primary Th2_resting dy | 0.8 | 93347_Small Airway | 33.7 |
| 4-6 in IL-2 | | Epithelium_none | |
| 93567primary Tr1_resting dy | 0.5 | 93348_Small Airway | 100.0 |
| 4-6 in IL-2 | | Epithelium_TNFa (4 ng/ml) | |
| | | and IL1b (1 ng/ml) | |
| 93351_CD45RA CD4 | 2.3 | 92668_Coronery Artery | 15.7 |
| lymphocyte_anti-CD28/anti- | | SMC_resting | |
| CD3 | | | |
| 93352_CD45RO CD4 | 0.5 | 92669_Coronery Artery | 9.2 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.4 | 93107_astrocytes_resting | 8.8 |
| CD28/anti-CD3 | | | |
| 93353_chronic CD8 | 0.4 | 93108_astrocytesTNFa (4 | 28.5 |
| Lymphocytes 2ry_resting dy 4- | | ng/ml) and IL1b (1 ng/ml) | |
| 6 in IL-2 | | | |
| 93574_chronic CD8 | 0.4 | 92666_KU-812 | 0.4 |
| Lymphocytes 2ry_activated | | (Basophil)_resting | |
| CD3/CD28 | | | |
| 93354_CD4_none | 0.4 | 92667_KU-812 | 0.0 |
| | | (Basophil)_PMA/ionoycin | |
| 93252_Secondary | 0.5 | 93579_CCD1106 | 66.0 |
| Th1/Th2/Tr1_anti-CD95 CH11 | | (Keratinocytes)_none | |
| 93103_LAK cells_resting | 0.8 | 93580_CCD1106 | 7.4 |
| | | (Keratinocytes)_TNFa and | |
| | | IFNg** | |
| 93788_LAK cells_IL-2 | 0.7 | 93791_Liver Cirrhosis | 2.3 |
| 93787_LAK cells_IL-2 + IL-12 | 0.5 | 93792_Lupus Kidney | 1.3 |
| 93789_LAK cells_IL-2 + IFN | 0.8 | 93577_NCI-H292 | 62.8 |
| gamma | | | |
| 93790_LAK cells_IL-2 + IL-18 | 0.7 | 93358_NCI-H292_IL-4 | 67.4 |
| 93104_LAK | 0.2 | 93360_NCI-H292_IL-9 | 95.3 |
| cells_PMA/ionomycin and IL- | | | |
| 18 | | | |
| 93578_NK Cells IL-2_resting | 0.5 | 93359_NCI-H292_IL-13 | 58.6 |
| 93109_Mixed Lymphocyte | 0.7 | 93357_NCI-H292_IFN gamma | 69.7 |
| Reaction_Two Way MLR | | | |
| 93110_Mixed Lymphocyte | 0.2 | 93777_HPAEC_- | 8.6 |
| Reaction_Two Way MLR | | | |
| 93111_Mixed Lymphocyte | 0.3 | 93778_HPAEC_IL-1 beta/TNA | 1.4 |
| Reaction_Two Way MLR | | alpha | |
| 93112_Mononuclear Cells | 0.4 | 93254_Normal Human Lung | 0.4 |
| (PBMCs)_resting | | Fibroblast_none | |
| 93113_Mononuclear Cells | 1.0 | 93253_Normal Human Lung | 0.2 |
| (PBMCs)_PWM | | Fibroblast_TNFa (4 ng/ml) and | |
| | | IL-1b (1 ng/ml) | |
| 93114_Mononuclear Cells | 0.5 | 93257_Normal Human Lung | 0.5 |
| (PBMCs)_PHA-L | | Fibroblast_IL-4 | |
| 93249_Ramos (B cell)_none | 0.5 | 93256_Normal Human Lung | 0.5 |
| | | Fibroblast_IL-9 | |
| 93250_Ramos (B | 1.7 | 93255_Normal Human Lung | 0.3 |
| cell)_ionomycin | | Fibroblast_IL-13 | |
| 93349_B lymphocytes_PWM | 1.5 | 93258_Normal Human Lung | 0.4 |
| | | Fibroblast_IFN gamma | |
| 93350_B lymphoytes_CD40L | 1.8 | 93106_Dermal Fibroblasts | 22.4 |
| and IL-4 | | CCD1070_resting | |
| 92665_EOL-1 | 0.5 | 93361_Dermal Fibroblasts | 17.4 |
| (Eosinophil)_dbcAMP | | CCD1070_TNF alpha 4 ng/ml | |
| differentiated | | | |
| 93248_EOL-1 | | | |
| (Eosinophil)_dbcAMP/PMAion | 1.1 | 93105_Dermal Fibroblasts | 11.5 |
| omycin | | CCD1070_IL-1 beta 1 ng/ml | |
| 93356_Dendritic Cells_none | 0.5 | 93772_dermal fibroblast_IFN | 0.2 |
| | | gamma | |
| 93355_Dendritic Cells_LPS | 0.4 | 93771_dermal fibroblast_IL-4 | 0.3 |
| 100 ng/ml | | | |
| 93775_Dendritic Cells_anti- | 0.4 | 93260_IBD Colitis 2 | 0.0 |
| CD40 | | | |
| 93774_Monocytes_resting | 1.1 | 93261_IBD Crohns | 0.0 |
| 93776_Monocytes_LPS 50 | 1.0 | 735010_Colon_normal | 1.5 |
| ng/ml | | | |
| 93581_Macrophages_resting | 0.5 | 735019_Lung_none | 6.9 |
| 93582_Macrophages_LPS 100 | 0.2 | 64028-1_Thymus_none | 3.5 |
| ng/ml | | | |
| 93098_HUVEC | 8.0 | 64030-1_Kidney_none | 1.9 |
| (Endothelial)_none | | | |
| 93099_HUVEC | 5.9 | | |
| (Endothelial)_starved | | | |

Summary Panel 1.3D Ag3073 There is widespread expression of the NOV2c gene in many samples in this panel. The highest level of expression is detected in a gastric cancer cell line with a CT value of 25.6. High levels of expression are also detected in breast, ovarian, lung pancreas, colon and liver cancer cell lines. Thus, expression of the NOV2c gene could be used to distinguish these cancer types from other tissues.

Among tissues with metabolic activity, highest expression of the NOV2c gene is measured in the thyroid (CT=26.5). There is also more moderate expression in adipose, fetal liver, fetal heart, and fetal skeletal muscle. Significant but low expression of the NOV2c gene is detected in the pancreas, adrenal and pituitary glands, adult heart, adult skeletal muscle, and adult liver. Therefore, the NOV2c gene product may play a role in the pathogenesis and or treatment of disease in any or all of these tissues, including obesity and diabetes.

For tissues involved in the central nervous system, there is significant expression of the NOV2c gene at low levels in the amygdala, cerebellum, hippocampus, thalamus, cerebral cortex, and spinal cord. This gene, a homologue of Keratin-4, is most likely a structural component of the cytoskeleton, and may be expressed in glia. While glial scarring is a major inhibitor of CNS repair and regeneration, organisms that show pronounced CNS regeneration in response to axotomy often have keratin as a glial structural protein as opposed to anti-glial fibrillary acidic protein (GFAP; ref. 1 and 2). Therefore, downregulation of expression of the NOV2c gene product after CNS injury may decrease glial scarring and enhance wound repair in head and spinal cord trauma.

Summary Panel 2D Ag3073 Significant expression of the NOV2c gene is ubiquitous across all the samples in this panel. This result correlates well with the results from panel 1.3D, where expression is widespread among the samples derived from cancerous cell lines. Highest expression of the NOV2c gene occurs in thyroid cancer (CT=23.8). Expression of the NOV2c gene is also present at high levels in ovarian, breast and bladder cancer samples. Furthermore, the NOV2c gene appears to be overexpressed in cancers from the ovary, breast, bladder and one out of two thyroid cancer samples, when compared to normal adjacent tissue. The same pattern can be seen in the uterus, where the NOV2c gene appears to be overexpressed in the cancerous tissue (CT=28.2) as compared to the corresponding normal uterine tissue (CT=33.2). This expression profile suggests that expression of the NOV2c gene product could be used to distinguish thyroid, ovarian, breast, bladder and uteran cancers from other tissues and to diagnose these cancer types. In addition, therapeutic modulation of the expression of the NOV2c gene or the activity of its protein product, through the use of small molecule drugs or antibodies, may be beneficial in the treatment of thyroid, ovarian, bladder, breast or uteran cancers.

Summary Panel 2.2 Ag3073 Significant expression of the NOV2c gene product is widespread among the tissue samples in this panel, with the highest level occurring in kidney (CT=27.5) and breast cancers (CT=27.7). Thus, expression of the NOV2c gene could be used to distinguish these cancer types from other tissues and to diagnose the presence of breast and kidney cancers. In addition, therapeutic modulation of the expression of the NOV2c gene or its protein product through the application of small molecule drugs or antibodies may be beneficial in the treatment of kidney or breast cancers.

Summary Panel 3D Ag3073 Highest expression of the NOV2c gene is measured in cervical carcinoma (CT=21.5). High levels of expression are also detected in clusters of samples derived from gastric, bladder, ovarian, breast and lung cancer cell lines. Thus, expression of the NOV2c gene could be used to distinguish these samples from other tissue types. In addition, therapeutic modulation of the expression of this gene or the function of its protein product, through the use of small molecule drugs or antibodies, may be effective in the treatment of bladder, pancreatic, ovarian, gastric, or lung cancer.

Summary Panel 4D Ag3073 Expression of the NOV2c gene appears to be highest in tissue samples originating in the lung. The NOV2c transcript encodes for a cytokeratin 8 like protein that is expressed in bronchial epithelial cells. In this panel, the NOV2c gene product is expressed in small airway epithelium and is overexpressed in the same tissue after treatment with TNF-a and IL-1 (CT =23.5). The NOV2c gene is also highly expressed in the muco-epidermoid cell line, H292. Thus, therapeutic modulation of the expression of this gene or the activity of its gene product, through the use of small molecule drugs, antibodies or protein therapeutics, may be beneficial in the treatment of inflammatory lung diseases. In addition, the NOV2c gene is highly expressed in keratinocytes (CT 24.1) and is down regulated upon TNF-a and IL-1 b treatment. This suggests that antibodies designed against the protein encoded by the NOV2c gene could potentially be used to distinguish between non-activated and activated keratinocytes.

References

1. Robson J A, Geisert E E Jr. (1994) Expression of a keratin sulfate proteoglycan during development of the dorsal lateral geniculate nucleus in the ferret. J Comp Neurol. 340:349-60.

ABAKAN is a keratin sulfate proteoglycan that was identified in rat brain by monoclonal antibody TED15 (Geisert et al. [1992] Brain Res. 571:165-168). It blocks neuronal attachment and neurite outgrowth in culture, is associated with astrocytes, and marks the boundaries of areas in the developing rat brain (Geisert and Bidanset [1993] Dev. Brain Res., 75:163-173). In the present study TED15 was used to examine the distribution of ABAKAN during laminar development of the dorsal lateral geniculate nucleus in ferrets. This distribution was also compared with that of astrocytes as displayed with antibodies to GFAP. In the adult, TED15 and anti-glial fibrillary acidic protein (GFAP) labeling are similar. Both are fairly uniform in the nucleus although somewhat elevated near the optic tract and in the interlaminar zone between laminae A and A1. During development the pattern is quite different. At postnatal day 1 (P1), before lamination is evident, TED 15 and anti-GFAP labeling are light in the nucleus. By P10, when laminae are emerging, both are elevated in the A-A1 interlaminar zone and in the C laminae. At P18, when laminae are distinct, TED15 labels the A-A1 interlaminar zone, and it marks the borders between the ON and OFF leaflets within A and A1 (Stryker and Zahs [1983] J. Neurosci. 3:1943-1951). In comparison, anti-GFAP marks the interlaminar zone but not the ON/OFF leaflets. By 6 weeks the nucleus resembles the adult nucleus. These results show that ABAKAN marks the boundaries of the major functional subdivisions of the lateral geniculate nucleus in the developing ferret and suggest that it plays a role in lamination.

2.Merrick S E, Pleasure S J, Lurie D I, Pijak D S, Selzer M E, Lee V M. (1995) Glial cells of the lamprey nervous system contain keratin-like proteins. J Comp Neurol. 355:199-210.

Lamprey axons regenerate following spinal cord transection despite the formation of a glial scar. As we were unable to detect a lamprey homologue of glial fibrillary acidic protein (GFAP), a major constituent of astrocytes, we studied the composition of intermediate filament (IF) proteins of lamprey glia. Monoclonal antibodies (mAbs) were raised to lamprey spinal cord cytoskeletal extracts and these mAbs were characterized by using Western blotting and immunocytochemistry. On two-dimensional (2-D) Western blots, five of the mAbs detected three major IF polypeptides in the molecular weight (MW) range of 45-56 kD. Further studies were conducted to determine the relationship between the lamprey glial-specific antigen and other mammalian IF proteins. Antikeratin 8 antibody recognized two of the three polypeptides. Several of the glial-specific mAbs reacted with human keratins 8 and 18 on Western blots. Keratin-like immunoreactivity was found in all parts of the central and peripheral nervous systems in both larval and adult lampreys. The immunocytochemical staining patterns of glial-specific mAbs were indistinguishable on lamprey spinal cord sections. However, on brain sections, two distinct patterns were observed. A subset of mAbs stained only a few glial fibers in the brain, whereas others stained many more brain glia, particularly the ependymal cells. The former group of mAbs recognized only the two lower MW polypeptides on 2-D Western blots, but the latter group of mAbs recognized all three major IF polypeptides. This correlation is supported by the observation that the highest MW IF polypeptide has an increased level of expression in the brain relative to the spinal cord. Thus, in the lamprey, the glial cells of both spinal cord and brain express molecules similar to simple epithelial cytokeratins, but their IFs may contain these keratins in different stoichiometric proportions. The widespread presence in the lamprey of primitive glial cells containing keratin-like intermediate filaments may have significance for the extraordinary ability of lamprey spinal axons to regenerate.

NOV3

Expression of NOV3 was assessed using the primer-probe set Ag2438 described in Table 18. Results from RTQ-PCR runs are shown in Tables 19, 20, and 21.

TABLE 18

Probe Name Ag2438

| | | | | |
|---|---|---|---|---|
| | | | | Start |
| Primers | Sequences | TM | Length | Position |

| | | | | |
|---|---|---|---|---|
| Forward | 5′-CCCTGTGGTGCAAAGTACTG-3′ (SEQ ID NO: 51) | 59.2 | 20 | 340 |
| Probe | FAM-5′-CCCCAAGGTTTACCTGATGAGTACG-3′- (SEQ ID NO: 52) | 65.8 | 25 | 371 |
| | TAMRA | | | |
| Reverse | 5′-CGGAAGGTTGTGACAAAGG-3′ (SEQ ID NO: 53) | 59.1 | 19 | 396 |

TABLE 19
Panel 1.3D

| | Relative | | Relative |
|---|---|---|---|
| | Expression (%) | | Expression (%) |
| | 1.3dtm4252f_{—} | | 1.3dtm4252f_{—} |
| Tissue Name | ag2438 | Tissue Name | ag2438 |
| Liver adenocarcinoma | 0.0 | Kidney (fetal) | 0.8 |
| Pancreas | 0.0 | Renal ca. 786-0 | 0.0 |
| Pancreatic ca. CAPAN 2 | 0.0 | Renal ca. A498 | 3.3 |
| Adrenal gland | 2.2 | Renal ca. RXF 393 | 0.0 |
| Thyroid | 0.2 | Renal ca. ACHN | 0.0 |
| Salivary gland | 0.2 | Renal ca. UO-31 | 0.0 |
| Pituitary gland | 49.3 | Renal ca. TK-10 | 6.7 |
| Brain (fetal) | 0.7 | Liver | 0.0 |
| Brain (whole) | 0.6 | Liver (fetal) | 0.2 |
| Brain (amygdala) | 0.5 | Liver ca. (hepatoblast) HepG2 | 0.0 |
| Brain (cerebellum) | 2.5 | Lung | 0.0 |
| Brain (hippocampus) | 1.2 | Lung (fetal) | 0.8 |
| Brain (substantia nigra) | 0.0 | Lung ca. (small cell) LX-l | 0.0 |
| Brain (thalamus) | 0.3 | Lung ca. (small cell) NCI-H69 | 1.8 |
| Cerebral Cortex | 0.0 | Lung ca. (s.cell var.) SHP-77 | 100.0 |
| Spinal cord | 0.7 | Lung ca. (large cell)NCI-H460 | 1.4 |
| CNS ca. (glio/astro) U87-MG | 0.5 | Lung ca. (non-sm. cell) A549 | 0.0 |
| CNS ca. (glio/astro) U-118-MG | 0.0 | Lung ca. (non-s.cell) NCI-H23 | 2.3 |
| CNS ca. (astro) SW1783 | 0.0 | Lung ca (non-s.cell) HOP-62 | 0.0 |
| CNS ca.* (neuro; met) SK-N-AS | 2.5 | Lung ca. (non-s.cl) NCI-H522 | 0.0 |
| CNS ca. (astro) SF-539 | 0.0 | Lung ca. (squam.) SW 900- | 0.6 |
| CNS ca. (astro) SNB-75 | 1.0 | Lung ca. (squam.) NCI-H596 | 0.6 |
| CNS ca. (glio) SNB-19 | 0.0 | Mammary gland | 0.3 |
| CNS ca. (glio) U251 | 0.0 | Breast ca.* (p1. effusion) MCF-7 | 0.0 |
| CNS ca. (glio) SF-295 | 0.0 | Breast ca.* (pl.ef) MDA-MB-231 | 0.0 |
| Heart (fetal) | 0.2 | Breast ca.* (p1. effusion) T47D | 0.0 |
| Heart | 0.0 | Breast ca. BT-549 | 0.0 |
| Fetal Skeletal | 23.5 | Breast ca. MDA-N | 0.2 |
| Skeletal muscle | 0.2 | Ovary | 0.5 |
| Bone marrow | 0.2 | Ovarian ca. OVCAR-3 | 0.0 |
| Thymus | 1.3 | Ovarian ca. OVCAR-4 | 0.0 |
| Spleen | 0.1 | Ovarian ca. OVCAR-5 | 1.7 |
| Lymph node | 0.1 | Ovarian ca. OVCAR-8 | 0.0 |
| Colorectal | 0.2 | Ovarian ca. IGROV-1 | 0.0 |
| Stomach | 1.1 | Ovarian ca.* (ascites) SK-OV-3 | 0.0 |
| Small intestine | 1.1 | Uterus | 1.1 |
| Colon ca. SW480 | 0.0 | Placenta | 0.0 |
| Colon ca.* (SW480 met)SW620 | 0.0 | Prostate | 1.6 |
| Colon ca. HT29 | 0.0 | Prostate ca.* (bone met)PC-3 | 0.0 |
| Colon ca. HCT-116 | 0.0 | Testis | 1.0 |
| Colon ca. CaCo-2 | 0.0 | Melanoma Hs688(A).T | 0.0 |
| 83219 CC Well to Mod Diff | 0.7 | Melanoma* (met) Hs688(B).T | 0.0 |
| (ODO3866) | | | |
| Colon ca. HCC-2998 | 0.0 | Melanoma UACC-62 | 2.2 |
| Gastric ca.* (liver met) NCI-N87 | 0.3 | Melanoma M14 | 0.0 |
| Bladder | 0.4 | Melanoma LOX IMVI | 0.0 |
| Trachea | 0.3 | Melanoma* (met) SK-MEL-5 | 0.0 |
| Kidney | 1.0 | Adipose | 0.2 |

TABLE 20
Panel 2D

| | Relative | | Relative |
|---|---|---|---|
| | Expression (%) | | Expression (%) |
| | 2dtm4253f_{—} | | 2dtm4253f_{—} |
| Tissue Name | ag2438 | Tissue Name | ag2438 |
| Normal Colon GENPAK | 16.2 | Kidney NAT Clontech 8120608 | 2.1 |
| 061003 | | | |
| 83219 CC Well to Mod Duff | 2.2 | Kidney Cancer Clontech | 1.1 |
| (ODO3866) | | 8120613 | |
| 83220 CC NAT (ODO3866) | 0.7 | Kidney NAT Clontech 8120614 | 7.4 |
| 83221 CC Gr.2 rectosigmoid | 1.8 | Kidney Cancer Clontech | 17.9 |
| (ODO3868) | | 9010320 | |
| 83222 CC NAT (ODO3868) | 2.1 | Kidney NAT Clontech 9010321 | 5.5 |
| 83235 CC Mod Diff | 2.6 | Normal Uterus GENPAK | 0.7 |
| (ODO3920) | | 061018 | |
| 83236 CC NAT (ODO3920) | 3.7 | Uterus Cancer GENPAK | 5.0 |
| | | 064011 | |
| 83237 CC Gr.2 ascend colon | 7.7 | Normal Thyroid Clontech A+ | 0.0 |
| (ODO3921) | | 6570-1 | |
| 83238 CC NAT (ODO3921) | 1.9 | Thyroid Cancer GENPAK | 0.0 |
| | | 064010 | |
| 83241 CC from Partial | 4.3 | Thyroid Cancer INVITROGEN | 3.7 |
| Hepatectomy (ODO4309) | | A302152 | |
| 83242 Liver NAT (ODO4309) | 1.7 | Thyroid NAT INVITROGEN | 2.8 |
| | | A302153 | |
| 87472 Colon mets to lung | 0.7 | Normal Breast GENPAK | 1.6 |
| (OD04451-01) | | 061019 | |
| 87473 Lung NAT (OD04451- | 0.0 | 84877 Breast Cancer | 0.8 |
| 02) | | (ODO4566) | |
| Normal Prostate Clontech A+ | 11.0 | 85975 Breast Cancer | 1.9 |
| 6546-1 | | (OD04590-01) | |
| 84140 Prostate Cancer | 28.3 | 85976 Breast Cancer Mets | 2.1 |
| (OD04410) | | (OD04590-03) | |
| 84141 Prostate NAT | 43.5 | 87070 Breast Cancer Metastasis | 6.0 |
| (OD04410) | | (OD04655-05) | |
| 87073 Prostate Cancer | 9.2 | GENPAK Breast Cancer | 0.5 |
| (OD04720-01) | | 064006 | |
| 87074 Prostate NAT | 38.7 | Breast Cancer Res. Gen. 1024 | 3.1 |
| (OD04720-02) | | | |
| Normal Lung GENPAK 061010 | 4.1 | Breast Cancer Clontech | 20.6 |
| | | 9100266 | |
| 83239 Lung Met to Muscle | 1.6 | Breast NAT Clontech 9100265 | 4.1 |
| (OD04286) | | | |
| 83240 Muscle NAT | 0.4 | Breast Cancer INVITROGEN | 9.6 |
| (OD04286) | | A209073 | |
| 84136 Lung Malignant Cancer | 100.0 | Breast NAT INVITROGEN | 1.5 |
| (ODO3126) | | A2090734 | |
| 84137 Lung NAT (OD03126) | 1.2 | Normal Liver GENPAK | 0.0 |
| | | 061009 | |
| 84871 Lung Cancer (OD04404) | 24.1 | Liver Cancer GENPAK 064003 | 4.1 |
| 84872 Lung NAT (OD04404) | 0.3 | Liver Cancer Research Genetics | 0.0 |
| | | RNA 1025 | |
| 84875 Lung Cancer (OD04565) | 8.1 | Liver Cancer Research Genetics | 66.0 |
| | | RNA 1026 | |
| 84876 Lung NAT (OD04565) | 0.3 | Paired Liver Cancer Tissue | 0.8 |
| | | Research Genetics RNA 6004-T | |
| 85950 Lung Cancer (OD04237-01) | 15.2 | Paired Liver Tissue Research | 0.9 |
| | | Genetics RNA 6004-N | |
| 85970 Lung NAT (OD04237-02) | 1.1 | Paired Liver Cancer Tissue | 42.0 |
| | | Research Genetics RNA 6005-T | |
| 83255 Ocular Mel Met to Liver | 2.7 | Paired Liver Tissue Research | 0.0 |
| (OD04310) | | Genetics RNA 6005-N | |
| 83256 Liver NAT (0DO4310) | 1.4 | Normal Bladder GENPAK | 12.8 |
| | | 061001 | |
| 84139 Melanoma Mets to Lung | 44.8 | Bladder Cancer Research | 3.7 |
| (OD04321) | | Genetics RNA 1023 | |
| 84138 Lung NAT (OD04321) | 1.8 | Bladder Cancer INVITROGEN | 78.5 |
| | | A302173 | |
| Normal Kidney GENPAK | 3.2 | 87071 Bladder Cancer | 6.0 |
| 061008 | | (OD04718-01) | |
| 83786 Kidney Ca, Nuclear | 22.7 | 87072 Bladder Normal | 1.1 |
| grade 2 (OD04338) | | Adjacent (OD04718-03) | |
| 83787 Kidney NAT (OD04338) | 34.9 | Normal Ovary Res. Gen. | 1.1 |
| 83788 Kidney Ca Nuclear grade | 0.4 | Ovarian Cancer GENPAK | 76.3 |
| 1/2 (OD04339) | | 064008 | |
| 83789 Kidney NAT (OD04339) | 13.1 | 87492 Ovary Cancer | 1.6 |
| | | (OD04768-07) | |
| 83790 Kidney ca. Clear cell | 0.5 | 87493 Ovary NAT (OD04768- | 3.7 |
| type (OD04340) | | 08) | |
| 83791 Kidney NAT (OD04340) | 6.1 | Normal Stomach GENPAK | 4.2 |
| | | 061017 | |
| 83792 Kidney ca. Nuclear | 2.8 | Gastric Cancer Clontech | 0.0 |
| trade 3 (OD04348) | | 9060358 | |
| 83793 Kidney NAT (OD04348) | 8.8 | NAT Stomach Clontech | 0.9 |
| | | 9060359 | |
| 87474 Kidney Cancer | 39.5 | Gastric Cancer Clontech | 4.0 |
| (OD04622-01) | | 9060395 | |
| 87475 Kidney NAT (OD04622- | 0.4 | NAT Stomach Clontech | 1.4 |
| 03) | | 9060394 | |
| 85973 Kidney Cancer | 0.7 | Gastric Cancer Clontech | 5.0 |
| (OD04450-01) | | 9060397 | |
| 85974 Kidney NAT (OD04450- | 5.0 | NAT Stomach Clontech | 0.8 |
| 03) | | 9060396 | |
| Kidney Cancer Clontech | 7.6 | Gastric Cancer GENPAK | 7.6 |
| 8120607 | | 064005 | |

TABLE 21
Panel 4D

| | Relative | | Relative |
|---|---|---|---|
| | Expression (%) | | Expression (%) |
| | 4dtm4254f_{—} | | 4dtm4254f_{—} |
| Tissue Name | ag2438 | Tissue Name | ag2438 |
| 93768_Secondary Th1_anti- | 0.0 | 93100_HUVEC | 0.0 |
| CD28/anti-CD3 | | (Endothelial)_IL-1b | |
| 93769_Secondary Th2_anti- | 0.0 | 93779_HUVEC | 0.0 |
| CD28/anti-CD3 | | (Endothelial)_IFN gamma | |
| 93770_Secondary Tr1_anti- | 0.0 | 93102_HUVEC | 0.0 |
| CD28/anti-CD3 | | (Endothelial)_TNF alpha + IFN | |
| | | gamma | |
| 93573_Secondary Th1_resting | 0.0 | 93101_HUVEC | 0.0 |
| day 4-6 in IL-2 | | (Endothelial)_TNF alpha +IL4 | |
| 93572_Secondary Th2_resting | 0.0 | 93781_HUVEC | 0.0 |
| day 4-6 in IL-2 | | (Endothelial)_IL-11 | |
| 93571_Secondary Tr1_resting | 0.0 | 93583_Lung Microvascular | 5.8 |
| day 4-6 in IL-2 | | Endothelial Cells_none | |
| 93568_primary Th1_anti- | 0.0 | 93584_Lung Microvascular | 6.2 |
| CD28/anti-CD3 | | Endothelial Cells_TNFa (4 | |
| | | ng/ml) and IL1b (1 ng/ml) | |
| 93569_primary Th2_anti- | 0.0 | 92662_Microvascular Dermal | 2.4 |
| CD28/anti-CD3 | | endothelium_none | |
| 93570_primary Tr1_anti- | 0.0 | 92663_Microsyasular Dermal | 5.3 |
| CD28/anti-CD3 | | endothelium_TNFa (4 ng/ml) | |
| | | and IL1b (1 ng/ml) | |
| 93565_primary Th1_resting dy | 0.0 | 93773_Bronchial | 28.9 |
| 4-6 in IL-2 | | epithelium_TNFa (4 ng/ml) and | |
| | | IL1b (1 ng/ml) ** | |
| 93566_primary Th2_resting dy | 0.0 | 93347_Small Airway | 3.0 |
| 4-6 in IL-2 | | Epithelium_none | |
| 93567_primary Tr1_resting dy | 0.0 | 93348_Small Airway | 17.2 |
| 4-6 in IL-2 | | Epithelium_TNFa (4 ng/ml) | |
| | | and IL1b (1 ng/ml) | |
| 93351_CD45RA CD4 | 0.0 | 92668_Coronery Artery | 15.4 |
| lymphocyte_anti-CD28/anti- | | SMC_resting | |
| CD3 | | | |
| 93352_CD45RO CD4 | 0.0 | 92669 _Coronery Artery | 21.0 |
| lymphocyte_anti-CD28/anti- | | SMC_TNFa (4 ng/ml) and IL1b | |
| CD3 | | (1 ng/ml) | |
| 93251_CD8 Lymphocytes_anti- | 0.0 | 93107_astrocytes_resting | 13.8 |
| CD28/anti-CD3 | | | |
| 93353_chronic CD8 | 0.0 | 93108_astrocytes_TNFa (4 | 5.5 |
| Lymphocytes 2ry_resting dy 4- | | g/ml) and IL1b (1 ng/ml) | |
| 6 in IL-2 | | | |
| 93574_chronic CD8 | 0.0 | 92666_KU-812 | 0.0 |
| Lymphocytes 2ry_activated | | (Basophil)_resting | |
| CD3/CD28 | | | |
| 93354_CD4_none | 0.0 | 92667_KU-812 | 0.0 |
| | | (Basophil)_PMA/ionoycin | |
| 93252_Secondary | 0.0 | 93579_CCD1106 | 100.0 |
| Th1/Th2/Tr1_anti-CD95 CH11 | | (Keratinocytes)_none | |
| 93103_LAX cells_resting | 0.0 | 93580_CCD1106 | 60.7 |
| | | (Keratinocytes)_TNFa and | |
| | | IFNg ** | |
| 93788_LAK cells_IL-2 | 0.0 | 93791_Liver Cirrhosis | 0.7 |
| 93787_LAK cells_IL-2 + IL-12 | 0.0 | 93792_Lupus Kidney | 3.9 |
| 93789_LAK cells_IL-2 + IFN | 0.0 | 93577_NCI-H292 | 0.0 |
| gamma | | | |
| 93790_LAK cells_IL-2 + IL-18 | 0.3 | 93358_NCI-H292_IL-4 | 0.0 |
| 93104_LAK | 0.0 | 93360_NCI-H292_IL-9 | 0.0 |
| cells_PMA/ionomycin and IL-18 | | | |
| 93578_NK Cells IL-2_resting | 0.0 | 93359_NCI-H292_IL-13 | 0.0 |
| 93109_Mixed Lymphocyte | 0.0 | 93357_NCI-H292_IFN gamma | 0.0 |
| Reaction_Two Way MLR | | | |
| 93110_Mixed Lymphocyte | 0.0 | 93777_HPAEC_- | 0.0 |
| Reaction_Two Way MLR | | | |
| 93111_Mixed Lymphocyte | 0.0 | 93778_HPAEC_IL-1 beta/TNA | 0.6 |
| Reaction_Two Way MLR | | alpha | |
| 93112_Mononuclear Cells | 0.0 | 93254_Normal Human Lung | 0.0 |
| (PBMCs)_resting | | Fibroblast_none | |
| 93113_Mononuclear Cells | 0.0 | 93253_Normal Human Lung | 1.9 |
| (PBMCs)_PWM | | Fibroblast_TNFa (4 ng/ml) and | |
| | | IL-1b (1 ng/ml) | |
| 93114_Mononuclear Cells | 0.0 | 93257_Normal Human Lung | 0.0 |
| (PBMCs)_PHA-L | | Fibroblast_IL-4 | |
| 93249_Ramos (B cell)_none | 0.0 | 93256_Normal Human Lung | 0.4 |
| | | Fibroblast_IL-9 | |
| 93250_Ramos (B | 0.0 | 93255_Normal Human Lung | 0.0 |
| cell)_ionomycin | | Fibroblast_IL-13 | |
| 93349_B lymphocytes_PWM | 0.0 | 93258_Normal Human Lung | 0.0 |
| | | Fibroblast_IFN gamma | |
| 93350_B lymphoytes_CD40L | 0.0 | 93106_Dermal Fibroblasts | 0.0 |
| and IL-4 | | CCD1070_resting | |
| 92665_EOL-1 | 0.0 | 93361_Dermal Fibroblasts | 0.0 |
| (Eosinophil)_dbcAMP | | CCD1070_TNF alpha 4 ng/ml | |
| differentiated | | | |
| 93248_EOL-1 | 0.0 | 93105_Dermal Fibroblasts | 0.4 |
| (Eosinophil)_dbcAMP/PMAion | | CCD1070_IL-1 beta 1 ng/ml | |
| omycin | | | |
| 93356_Dendritic Cells_none | 0.0 | 93772_dermal fibroblast_IFN | 0.0 |
| | | gamma | |
| 93355_Dendritic Cells_LPS | 0.0 | 93771_dermal fibroblast_IL-4 | 0.0 |
| 100 ng/ml | | | |
| 93775_Dendritic Cells_anti- | 0.0 | 93260_IBD Colitis 2 | 0.0 |
| CD40 | | | |
| 93774_Monocytes_resting | 0.0 | 93261_IBD Crohns | 1.2 |
| 93776_Monocytes_LPS 50 | 0.0 | 735010_Colon_normal | 2.4 |
| ng/ml | | | |
| 93581_Macrophages_resting | 0.0 | 735019_Lung_none | 4.4 |
| 93582_Macrophages_LPS 100 | 0.0 | 64028-1_Thymus_none | 19.2 |
| ng/ml | | | |
| 93098_HUVEC | 0.0 | 64030-1_Kidney_none | 26.4 |
| (Endothelial)_none | | | |
| 93099_HUVEC | 0.0 | | |
| (Endothelial)_starved | | | |

Summary Panel 1.3D Ag2438 The NOV3 gene is highly expressed in lung cancer (CT=25.6), the pituitary gland (CT=26.6), and fetal skeletal muscle (CT=27.7). Thus, expression of this gene could be used to distinguish between lung cancer cell lines and other cell lines or tissue types and between pituitary gland and fetal skeletal muscle and other tissue types.

Among metabolically active tissues, expression is observed in the adrenal gland, adipose, fetal heart (CT=34.4), fetal skeletal muscle (CT=27.7), and fetal liver (CT=34.3). In contrast, expression of the NOV3 gene is unobservable in adult heart and liver, and low in adult skeletal muscle (CT=34.8). The difference in expression levels of the NOV3 gene between fetal and adult tissues suggests that this gene could be used to distinguish betweent the two types of tissues. In addition, the overexpression of the NOV3 gene in fetal skeletal muscle as opposed to adult skeletal muscle suggests that the protein product may enhance muscular growth or development in the fetus and thus may also act in a regenerative capacity in the adult. Therefore, therapeutic modulation of the NOV3 gene could be useful in treatment of muscle-related disease. More specifically, treatment of weak or dystrophic muscle with the protein encoded by this gene could restore muscle mass or function.

For tissues involved in the central nervous system, expression of the NOV3 gene is expressed in the pituitary gland, cerebellum, and hippocampus, and at low levels in the thalamus, amygdala and the fetal brain. The hippocampus is a primary brain region involved in Alzheimer's disease. Studies indicate that collagen and the enzymes that affect collagen stability may play a role in Alzheimer's disease-asociated processes, such as amyloid-beta deposition, APP function and blood brain barrier dysfunction (ref. 1-4). Thus, agents that target the NOV3 gene product may be useful in the treatment of Alzheimer's disease.

The NOV3 gene is also expressed in fetal lung (CT=32.6) but not in adult lung, and in normal prostate (CT=31.5) and not in prostate cancer cell lines. These results suggest that expression of the gene could be used to distinguish between fetal and adult lung tissue and between normal and cancerous prostate tissue. In addition, the preferential expression of the NOV3 gene in fetal tissue (lung, skeletal muscle, liver and heart) suggests that this gene or its protein product may be involved in tissue development. Thus, therapeutic modulation of the NOV3 gene or its protein product could be effective in the treatment of tissues affected by degenerative diseases.

Summary Panel 2D Ag2438 Significant expression of the NOV3 gene is measured in lung cancer (CT=28.5). Expression of the NOV3 gene is also detectable at low but significant levels in liver, ovarian, uterine and bladder cancers. In addition, the NOV3 gene appears to be overexpressed in the cancerous specimens when compared to normal adjacent tissue. Thus, expression of this gene could be used to distinguish between normal tissues and uterine, ovarian, liver, bladder and lung cancers.

Summary Panel 4D Ag2438 The NOV3 gene is significantly expressed in keratinocytes (CT=28.5). It is expressed at lower levels in both bronchial epithelium and small airway epithelium after treatment with TNF-a and IL-1b. This expression profile may reflect a repair mechanism process following injury. Therefore, expression of the NOV3 gene could potentially be used as marker of stressed epithelium in the lung and the skin. In addition, therapeutic modulation of the expression or activity of the NOV3 gene product, through the use of small molecule drugs or antibodies, may be effective in the treatment lung and skin inflammatory diseases.

References

1. Leake A, Morris C M, Whateley J. (2000) Brain matrix metalloproteinase 1 levels are elevated in Alzheimer's disease. Neurosci Lett. 291:201-3.

Several lines of evidence indicate that there may be an inflammatory component to the pathology of Alzheimer's disease (AD), the major form of degenerative dementia in the elderly. Activity of inflammatory cells, and the elaboration of toxic molecules by such cells may be a significant factor in disease progression. In peripheral inflammatory states, the increased activity of matrix metalloproteinase (MMP) enzymes are a major cause of tissue breakdown and secondary damage in diseases such as rheumatoid arthritis. The activity of such enzymes in the normal or diseased central nervous system is, however, not well characterized. We have therefore determined the levels of MMP 1 (collagenase) in the normal human brain and in AD. MMP1 levels were relatively low though were significantly elevated by approximately 50% in AD in all cortical areas examined. Given the activity towards collagen of MMP1, it is possible that enhanced MMP1 activity in AD, may contribute to the blood-brain barrier dysfunction seen in AD.

2. Brown W R, Moody D M, Thore C R, Challa V R. (2000) Cerebrovascular pathology in Alzheimer's disease and leukoaraiosis Ann N Y Acad Sci. 903:39-45.

A high percentage of patients with Alzheimer's disease (AD) show evidence of white matter degeneration known as leukoaraiosis (LA), which is due to chronic ischemia. We found that the periventricular veins tend to become occluded by multiple layers of collagen in the vessel walls in the elderly. This collagen deposition is particularly excessive in LA lesions. Therefore, it is present in the brains of many AD patients, along with other ischemia-causing cerebrovascular pathology. We found evidence that there is severe loss of oligodendrocytes in LA, due to extensive apoptosis. No evidence of inflammation was found in the LA lesions. In thick celloidin sections of AD brain, we have obtained detailed 3D views of small (early) deposits of amyloid (stained with beta-amyloid antibody) around capillaries (stained with collagen IV antibody).

3. Armstrong R A, Cairns N J, Lantos P L. (1998) Spatial distribution of diffuse, primitive, and classic amyloid-beta deposits and blood vessels in the upper laminae of the frontal cortex in Alzheimer disease Alzheimer Dis Assoc Disord. 12:378-83.

The spatial distribution of the diffuse, primitive, and classic amyloid-beta deposits was studied in the upper laminae of the superior frontal gyrus in cases of sporadic Alzheimer disease (AD). Amyloid-beta-stained tissue was counterstained with collagen IV to determine whether the spatial distribution of the amyloid-beta deposits along the cortex was related to blood vessels. In all patients, amyloid-beta deposits and blood vessels were aggregated into distinct clusters and in many patients, the clusters were distributed with a regular periodicity along the cortex. The clusters of diffuse and primitive deposits did not coincide with the clusters of blood vessels in most patients. However, the clusters of classic amyloid-beta deposits coincided with those of the large diameter (>10 microm) blood vessels in all patients and with clusters of small-diameter (<10 microm) blood vessels in four patients. The data suggest that, of the amyloid-beta subtypes, the clusters of classic amyloid-beta deposits appear to be the most closely related to blood vessels and especially to the larger-diameter, vertically penetrating arterioles in the upper cortical laminae.

4. Coulson E J, Barrett G L, Storey E, Bartlett P F, Beyreuther K, Masters C L. (1997) Down-regulation of the amyloid protein precursor of Alzheimer's disease by antisense oligonucleotides reduces neuronal adhesion to specific substrata Brain Res. 770:72-80.

The hallmark of Alzheimer's disease is the cerebral deposition of amyloid which is derived from the amyloid precursor protein (APP). The function of APP is unknown but there is increasing evidence for the role of APP in cell-cell and/or cell-matrix interactions. Primary cultures of murine neurons were treated with antisense oligonucleotides to down-regulate APP. This paper presents evidence that APP mediates a substrate-specific interaction between neurons and extracellular matrix components collagen type I, laminin and heparan sulphate proteoglycan but not fibronectin or poly-L-lysine. It remains to be determined whether this effect is the direct result of APP-matrix interactions, or whether an intermediatry pathway is involved.

NOV4

Expression of gene NOV4 was assessed using the primer-probe sets Ag2429 and Ag1504, described in Tables 22 and 23. Results from RTQ-PCR runs are shown in Tables 24, 25, 26,27, and 28.

TABLE 22

Probe Name Ag2429

| | | | | |
|---|---|---|---|---|
| | | | | Start |
| Primers | Sequences | TM | Length | Position |

| | | | | |
|---|---|---|---|---|
| Forward | 5′-TGGTCACAGGGACAAACTTC-3′ (SEQ ID NO: 54) | 58.5 | 20 | 292 |
| Probe | TET-5′-CGTTGCTGATAACATCGTATACTTCCA-3′-TAMRA | 64.3 | 27 | 313 |
| | (SEQ ID NO: 55) | | | |
| Reverse | 5′-GGTCAAGGGCTTGTTTTCAT-3′ (SEQ ID NO: 56) | 59 | 20 | 361 |

TABLE 23

Probe Name Ag15O4

| | | | | |
|---|---|---|---|---|
| | | | | Start |
| Primers | Sequences | TM | Length | Position |

| | | | | |
|---|---|---|---|---|
| Forward | 5′-ATCTCAGCATCCTTGGTACCTT-3′ (SEQ ID NO: 57) | 59.1 | 22 | 6 |
| Probe | FAM-5′-CAACTCTCTGGTCCTTTCTGCCCTGT-3′-TAMRA | 68.9 | 26 | 41 |
| | (SEQ ID NO: 58) | | | |
| Reverse | 5′-ACACGTCATCGTGGTAGCA-3′ (SEQ ID NO: 59) | 58.7 | 19 | 77 |

TABLE 24
Panel 1.2

| | Relative | | Relative |
|---|---|---|---|
| | Expression (%) | | Expression (%) |
| | 1.2tm2122f_{—} | | 1.2tm2122f_{—} |
| Tissue Name | ag1504 | Tissue Name | ag1504 |
| Endothelial cells | 55.5 | Renal ca. 786-0 | 2.4 |
| Heart (fetal) | 4.7 | Renal ca. A498 | 8.0 |
| Pancreas | 12.3 | Renal ca. RXF 393 | 4.4 |
| Pancreatic ca. CAPAN 2 | 6.7 | Renal ca. ACHN | 11.2 |
| Adrenal Gland (new lot*) | 38.2 | Renal ca. UO-31 | 41.2 |
| Thyroid | 1.2 | Renal ca. TK-l0 | 19.2 |
| Salivary gland | 30.6 | Liver | 13.2 |
| Pituitary gland | 3.6 | Liver (fetal) | 5.2 |
| Brain (fetal) | 0.5 | Liver ca. (hepatoblast) HepG2 | 12.5 |
| Brain (whole) | 0.6 | Lung | 2.4 |
| Brain (amygdala) | 4.8 | Lung (fetal) | 0.7 |
| Brain (cerebellum) | 8.5 | Lung ca. (small cell) LX-l | 19.2 |
| Brain (hippocampus) | 16.2 | Lung ca. (small cell) NCI-H69 | 14.5 |
| Brain (thalamus) | 5.7 | Lung ca. (s.cell var.) SHP-77 | 6.4 |
| Cerebral Cortex | 37.1 | Lung ca. (large cell) NCI-H460 | 34.2 |
| Spinal cord | 1.9 | Lung ca. (non-sm. cell) A549 | 13.2 |
| CNS ca. (glio/astro) U87-MG | 66.0 | Lung ca. (non-s.cell) NCI-H23 | 31.4 |
| CNS ca. (glio/astro) U-118-MG | 25.5 | Lung ca (non-s.cell) HOP-62 | 65.5 |
| CNS ca. (astro) SW1783 | 5.2 | Lung ca. (non-s.cl) NCI-H522 | 21.8 |
| CNS ca.* (neuro; met) 5K-N-AS | 3.1 | Lung ca. (squam.) SW 900 | 15.0 |
| CNS ca. (astro) SF-539 | 11.6 | Lung ca. (squam.) NCI-H596 | 15.8 |
| CNS ca. (astro) SNB-75 | 14.3 | Mammary gland | 7.9 |
| CNS ca. (glio) SNB-19 | 19.1 | Breast ca.* (pl. effusion) MCF-7 | 20.4 |
| CNS ca. (glio) U251 | 6.2 | Breast ca.* (pl.ef) MDA-MB-231 | 3.4 |
| CNS ca. (glio) SF-295 | 10.4 | Breast ca.* (pl. effusion) T47D | 29.9 |
| Heart | 46.7 | Breast ca. BT-549 | 17.9 |
| Skeletal Muscle (new lot*) | 43.8 | Breast ca. MDA-N | 6.8 |
| Bone marrow | 26.4 | Ovary | 8.0 |
| Thymus | 1.3 | Ovarian ca. OVCAR-3 | 14.5 |
| Spleen | 6.8 | Ovarian ca. OVCAR-4 | 32.5 |
| Lymph node | 2.1 | Ovarian ca. OVCAR-5 | 59.5 |
| Colorectal | 21.3 | Ovarian ca. OVCAR-8 | 26.4 |
| Stomach | 3.3 | Ovarian ca. IGROV-1 | 70.7 |
| Small intestine | 29.7 | Ovarian ca.* (ascites) SK-OV-3 | 33.4 |
| Colon ca. SW480 | 9.8 | Uterus | 5.2 |
| Colon ca.* (SW480 met)SW620 | 15.4 | Placenta | 10.8 |
| Colon ca. HT29 | 3.1 | Prostate | 30.6 |
| Colon ca. HCT-116 | 13.7 | Prostate ca.* (bone met)PC-3 | 36.3 |
| Colon ca. CaCo-2 | 9.4 | Testis | 2.0 |
| 83219 CC Well to Mod Duff | 3.9 | Melanoma Hs688(A).T | 6.0 |
| (ODO3866) | | | |
| Colon ca. HCC-2998 | 37.9 | Melanoma* (met) Hs688(B).T | 3.8 |
| Gastric ca.* (liver met) NCI-N87 | 31.6 | Melanoma UACC-62 | 9.7 |
| Bladder | 100.0 | Melanoma M14 | 7.7 |
| Trachea | 0.8 | Melanoma LOX IMVI | 4.2 |
| Kidney | 31.0 | Melanoma* (met) SK-MEL-5 | 6.6 |
| Kidney (fetal) | 25.9 | | |

TABLE 25
Panel 1.3D

| | Relative | | Relative |
|---|---|---|---|
| | Expression (%) | | Expression (%) |
| | 1.3dtm4257t_{—} | | 1.3dtm4257t_{—} |
| Tissue Name | ag2429 | Tissue Name | ag2429 |
| Liver adenocarcinoma | 34.2 | Kidney (fetal) | 2.4 |
| Pancreas | 6.6 | Renal ca. 786-0 | 1.4 |
| Pancreatic ca. CAPAN 2 | 11.3 | Renal ca. A498 | 20.3 |
| Adrenal gland | 17.4 | Renal ca. RXF 393 | 1.5 |
| Thyroid | 5.3 | Renal ca. ACIIN | 2.0 |
| Salivary gland | 12.5 | Renal ca. UO-31 | 0.0 |
| Pituitary gland | 19.2 | Renal ca. TK-10 | 3.5 |
| Brain (fetal) | 11.0 | Liver | 2.4 |
| Brain (whole) | 12.6 | Liver (fetal) | 12.2 |
| Brain (amygdala) | 23.5 | Liver ca. (hepatoblast) HepG2 | 11.8 |
| Brain (cerebellum) | 2.5 | Lung | 19.1 |
| Brain (hippocampus) | 95.3 | Lung (fetal) | 7.6 |
| Brain (substantia nigra) | 5.6 | Lung ca. (small cell) LX-1 | 8.8 |
| Brain (thalamus) | 33.4 | Lung ca. (small cell) NCI-H69 | 6.4 |
| Cerebral Cortex | 19.3 | Lung ca. (s.cell var.) SHP-77 | 17.4 |
| Spinal cord | 13.1 | Lung ca. (large cell) NCI-H460 | 6.1 |
| CNS ca. (glio/astro) U87-MG | 33.0 | Lung ca. (non-sm. cell) A549 | 5.9 |
| CNS ca. (glio/astro) U-118-MG | 58.6 | Lung ca. (non-s.cell) NCI-H23 | 42.3 |
| CNS ca. (astro) SW1 783 | 13.6 | Lung ca (non-s.cell) HOP-62 | 16.8 |
| CNS ca.* (neuro; met) SK-N- | 39.2 | Lung ca. (non-s.d) NCI-H522 | 4.0 |
| AS | | | |
| CNS ca. (astro) SF-539 | 18.4 | Lung ca. (squam.) SW 900 | 2.2 |
| CNS ca. (astro) SNB-75 | 11.2 | Lung ca. (squam.) NCI-H596 | 2.9 |
| CNS ca. (glio) SNB-19 | 12.2 | Mammary gland | 16.2 |
| CNS ca. (glio) U251 | 11.3 | Breast ca.* (pl. effusion) MCF-7 | 15.9 |
| CNS ca. (glio) SF-295 | 21.3 | Breast ca.* (pl.ef) MDA-MB- | 46.0 |
| | | 231 | |
| Heart (fetal) | 1.9 | Breast ca.* (pl. effusion) T47D | 8.2 |
| Heart | 4.1 | Breast ca. BT-549 | 100.0 |
| Fetal Skeletal | 45.7 | Breast ca. MDA-N | 4.8 |
| Skeletal muscle | 5.3 | Ovary | 3.2 |
| Bone marrow | 77.4 | Ovarian ca. OVCAR-3 | 13.1 |
| Thymus | 12.9 | Ovarian ca. OVCAR-4 | 3.6 |
| Spleen | 27.0 | Ovarian ca. OVCAR-5 | 6.4 |
| Lymph node | 21.9 | Ovarian ca. OVCAR-8 | 18.2 |
| Colorectal | 24.1 | Ovarian ca. IGROV-1 | 16.5 |
| Stomach | 19.1 | Ovarian ca.* (ascites) SK-OV-3 | 20.2 |
| Small intestines | 24.3 | Uterus | 12.7 |
| Colon ca. SW480 | 1.6 | Placenta | 10.9 |
| Colon ca.* (SW480 met) SW620 | 11.3 | Prostate | 7.8 |
| Colon ca. HT29 | 4.5 | Prostate ca.* (bone met) PC-3 | 11.6 |
| Colon ca. HCT-116 | 9.6 | Testis | 11.8 |
| Colon ca. CaCo-2 | 5.1 | Melanoma Hs688 (A) .T | 0.7 |
| 83219 CC Well to Mod Diff | 8.8 | Melanoma* (met) Hs688 (B) .T | 1.6 |
| (ODO3866) | | | |
| Colon ca. HCC-2998 | 33.0 | Melanoma UACC-62 | 1.6 |
| Gastric ca.* (liver met) NCI- | 39.2 | Melanoma M14 | 2.3 |
| N87 | | | |
| Bladder | 21.3 | Melanoma LOX IMVI | 12.1 |
| Trachea | 35.8 | Melanoma* (met) SK-MEL-5 | 4.0 |
| Kidney | 3.3 | Adipose | 13.6 |

TABLE 26
Panel 2D

| | Relative | Relative | |
|---|---|---|---|
| | Expression (%) | Expression (%) | |
| | 2dtm4236t_{—} | 2Dtm2344f_{—} | 2Dtm2412f_{—} |
| Tissue Name | ag2429 | ag1504 | ag1504 |
| Normal Colon GENPAK 061003 | 54.0 | 64.2 | 24.3 |
| 83219 CC Well to Mod Diff (ODO3866) | 5.3 | 13.4 | 4.2 |
| 83220 CC NAT (ODO3866) | 7.7 | 13.3 | 3.1 |
| 83221 CC Gr.2 rectosigmoid (ODO3868) | 9.2 | 17.2 | 10.8 |
| 83222 CC NAT (ODO3868) | 4.6 | 7.4 | 2.5 |
| 83235 CC Mod Diff (ODO3920) | 18.6 | 20.9 | 30.8 |
| 83236 CC NAT (ODO3920) | 9.3 | 24.7 | 8.2 |
| 83237 CC Gr.2 ascend colon (ODO3921) | 19.1 | 15.2 | 10.5 |
| 83238 CC NAT (ODO3921) | 4.5 | 5.7 | 2.0 |
| 83241 CC from Partial Hepatectomy | 7.1 | 4.6 | 6.2 |
| (ODO4309) | | | |
| 83242 Liver NAT (ODO4309) | 6.0 | 2.5 | 5.2 |
| 87472 Colon mets to lung (ODO4451-01) | 10.2 | 17.6 | 26.8 |
| 87473 Lung NAT (ODO445 1-02) | 14.3 | 15.8 | 3.7 |
| Normal Prostate Clontech A + 6546-1 | 6.2 | 30.6 | 30.6 |
| 84140 Prostate Cancer (ODO4410) | 21.5 | 33.4 | 28.7 |
| 84141 Prostate NAT (ODO4410) | 21.8 | 25.3 | 51.4 |
| 87073 Prostate Cancer (ODO4720-01) | 39.5 | 30.8 | 72.2 |
| 87074 Prostate NAT (ODO4720-02) | 29.5 | 29.9 | 52.8 |
| Normal Lung GENPAK 061010 | 91.4 | 66.0 | 100.0 |
| 83239 Lung Met to Muscle (ODO4286) | 10.8 | 9.2 | 18.9 |
| 83240 Muscle NAT (ODO4286) | 11.1 | 46.0 | 47.6 |
| 84136 Lung Malignant Cancer (ODO3126) | 6.8 | 28.9 | 40.3 |
| 84137 Lung NAT (ODO3126) | 26.2 | 20.6 | 46.7 |
| 84871 Lung Cancer (ODO4404) | 9.0 | 9.0 | 7.6 |
| 84872 Lung NAT (ODO4404) | 10.7 | 21.5 | 11.5 |
| 84875 Lung Cancer (ODO4565) | 11.8 | 6.0 | 17.7 |
| 84876 Lung NAT (ODO4565) | 14.9 | 31.4 | 59.5 |
| 85950 Lung Cancer (ODO4237-01) | 13.6 | 7.0 | 14.8 |
| 85970 Lung NAT (ODO4237-02) | 21.6 | 35.8 | 9.9 |
| 83255 Ocular Mel Met to Liver (ODO4310) | 5.5 | 11.5 | 16.8 |
| 83256 LiverNAT (ODO4310) | 6.0 | 8.7 | 11.2 |
| 84139 Melanoma Mets to Lung (ODO4321) | 4.8 | 8.6 | 5.8 |
| 84138 Lung NAT (ODO4321) | 9.9 | 15.3 | 37.4 |
| Normal Kidney GENPAK 061008 | 39.5 | 56.6 | 54.7 |
| 83786 Kidney Ca, Nuclear grade 2 | 11.5 | 44.4 | 39.8 |
| (ODO4338) | | | |
| 83787 Kidney NAT (ODO4338) | 11.5 | 12.9 | 9.9 |
| 83788 Kidney Ca Nuclear grade ½ | 34.4 | 37.6 | 20.2 |
| (ODO4339) | | | |
| 83789 Kidney NAT (ODO4339) | 4.7 | 12.9 | 13.4 |
| 83790 Kidney ca. Clear cell type (ODO4340) | 24.5 | 34.6 | 54.0 |
| 83791 Kidney NAT (ODO4340) | 17.3 | 27.9 | 22.8 |
| 83792 Kidney Ca, Nuclear grade 3 | 1.4 | 5.2 | 12.1 |
| (ODO4348) | | | |
| 83793 Kidney NAT (ODO4348) | 11.2 | 34.6 | 46.0 |
| 87474 Kidney Cancer (ODO4622-01) | 11.1 | 16.8 | 50.3 |
| 87475 Kidney NAT (ODO4622-03) | 2.1 | 8.2 | 8.3 |
| 85973 Kidney Cancer (ODO4450-01) | 2.5 | 21.5 | 25.2 |
| 85974 Kidney NAT (ODO4450-03) | 8.1 | 11.6 | 20.4 |
| Kidney Cancer Clontech 8120607 | 1.0 | 0.0 | 0.5 |
| Kidney NAT Clontech 8120608 | 0.0 | 5.4 | 2.3 |
| Kidney Cancer Clontech 8120613 | 6.2 | 19.6 | 24.1 |
| Kidney NAT Clontech 8120614 | 3.0 | 6.0 | 4.0 |
| Kidney Cancer Clontech 9010320 | 16.0 | 31.6 | 9.9 |
| Kidney NAT Clontech 9010321 | 15.8 | 15.5 | 14.0 |
| Normal Uterus GENPAK 061018 | 9.5 | 13.7 | 26.6 |
| Uterus Cancer GENPAK 064011 | 35.8 | 60.7 | 94.0 |
| Normal Thyroid Clontech A + 6570-1 | 6.0 | 34.2 | 18.2 |
| Thyroid Cancer GENPAK 064010 | 5.6 | 5.2 | 21.9 |
| Thyroid Cancer INVITROGEN A302152 | 10.4 | 18.0 | 27.2 |
| Thyroid NAT INVITROGEN A302153 | 13.6 | 18.7 | 26.8 |
| Normal Breast GENPAK 061019 | 42.0 | 51.0 | 54.0 |
| 84877 Breast Cancer (ODO4566) | 11.7 | 18.7 | 53.6 |
| 85975 Breast Cancer (ODO4590-01) | 18.9 | 19.3 | 12.1 |
| 85976 Breast Cancer Mets (ODO4590-03) | 34.6 | 41.8 | 85.9 |
| 87070 Breast Cancer Metastasis (ODO4655- | 26.2 | 32.5 | 46.7 |
| 05 | | | |
| GENPAK Breast Cancer 064006 | 16.3 | 42.0 | 52.1 |
| Breast Cancer Res. Gen. 1024 | 29.5 | 63.7 | 23.8 |
| Breast Cancer Clontech 9100266 | 20.6 | 43.5 | 24.8 |
| Breast NAT Clontech 9100265 | 14.0 | 12.9 | 29.3 |
| Breast Cancer INVITROGEN A209073 | 19.8 | 21.2 | 45.4 |
| Breast NAT INVITROGEN A2090734 | 24.3 | 40.9 | 12.6 |
| Normal Liver GENPAK 061009 | 4.3 | 3.9 | 15.7 |
| Liver Cancer GENPAK 064003 | 6.4 | 29.1 | 2.2 |
| Liver Cancer Research Genetics RNA 1025 | 7.8 | 13.2 | 5.5 |
| Liver Cancer Research Genetics RNA 1026 | 100.0 | 2.1 | 2.5 |
| Paired Liver Cancer Tissue Research Genetics | 11.7 | 20.4 | 7.8 |
| RNA 6004-T | | | |
| Paired Liver Tissue Research Genetics RNA | 20.0 | 20.9 | 9.7 |
| 6004-N | | | |
| Paired Liver Cancer Tissue Research Genetics | 0.7 | 3.6 | 0.7 |
| RNA 6005-T | | | |
| Paired Liver Tissue Research Genetics RNA | 0.7 | 2.2 | 0.6 |
| 6005-N | | | |
| Normal Bladder GENPAK 061001 | 34.6 | 36.9 | 54.7 |
| Bladder Cancer Research Genetics RNA 1023 | 10.9 | 18.0 | 13.4 |
| Bladder Cancer INVITROGEN A302173 | 24.1 | 21.0 | 8.8 |
| 87071 Bladder Cancer (ODO4718-01) | 6.2 | 12.2 | 10.6 |
| 87072 Bladder Normal Adjacent (ODO4718- | 38.7 | 46.7 | 30.1 |
| 03) | | | |
| Normal Ovary Res. Gen. | 7.1 | 1.0 | 2.1 |
| Ovarian Cancer GENPAK 064008 | 29.7 | 18.2 | 14.1 |
| 87492 Ovary Cancer (ODO4768-07) | 84.1 | 100.0 | 51.8 |
| 87493 Ovary NAT (ODO4768-08) | 9.2 | 6.9 | 8.7 |
| Normal Stomach GENPAK 06107 | 21.0 | 19.2 | 18.0 |
| Gastric Cancer Clontech 9060358 | 2.2 | 4.5 | 1.0 |
| NAT Stomach Clontech 9060359 | 3.5 | 2.4 | 4.5 |
| Gastric Cancer Clontech 9060395 | 11.7 | 14.5 | 17.7 |
| NAT Stomach Clontech 9060394 | 11.9 | 5.6 | 5.1 |
| Gastric Cancer Clontech 9060397 | 20.7 | 5.4 | 12.8 |
| NAT Stomach Clontech 9060396 | 2.1 | 2.3 | 1.5 |
| Gastric Cancer GENPAK 064005 | 36.9 | 26.6 | 28.7 |

TABLE 27
Panel 4D

| | Relative | | Relative |
|---|---|---|---|
| | Expression (%) | | Expression (%) |
| | 4dtm4237t_{—} | | 4dtm4237t_{—} |
| Tissue Name | ag2429 | Tissue Name | ag2429 |
| 93768_Secondary Th1_anti- | 14.8 | 93100_HUVEC | 1.4 |
| CD28/anti-CD3 | | (Endothelial)_IL-1b | |
| 93769_Secondary Th2_anti- | 12.1 | 93779_HUVEC | 14.9 |
| CD28/anti-CD3 | | (Endothelial)_IFN gamma | |
| 93770_Secondary Tr1_anti- | 11.7 | 93102_HUVEC | 5.2 |
| CD28/anti-CD3 | | (Endothelial)_TNF alpha + IFN | |
| | | gamma | |
| 93573_Secondary Th1_resting | 5.5 | 93101_HUVEC | 6.4 |
| day 4-6 in IL-2 | | (Endothelial)_TNF alpha + 1L4 | |
| 93572_Secondary Th2_resting | 5.8 | 93781_HUVEC | 3.7 |
| day 4-6 in IL-2 | | (Endothelial)_IL-1l | |
| 93571_Secondary Tr1_resting | 10.1 | 93583_Lung Microvascular | 5.4 |
| day 4-6 in IL-2 | | Endothelial Cells_none | |
| 93568_primary Th1_anti- | 12.1 | 93584_Lung Microvascular | 7.0 |
| CD28/anti-CD3 | | Endothelial Cells_TNFa (4 | |
| | | ng/ml) and IL1b (1 ng/ml) | |
| 93569_primary Th2_anti- | 13.6 | 92662_Microvascular Dermal | 5.6 |
| CD28/anti-CD3 | | endothelium_none | |
| 93570_primary Tr1_anti- | 19.5 | 92663_Microsvasular Dermal | 6.0 |
| CD28/anti-CD3 | | endothelium_TNFa (4 ng/ml) | |
| | | and IL1b (1 ng/ml) | |
| 93565_primary Th1_resting dy | 30.8 | 93773_Bronchial | 5.1 |
| 4-6 in IL-2 | | epithelium_TNFa (4 ng/ml) and | |
| | | IL1b (1 ng/ml) ** | |
| 93566_primary Th2_resting dy | 14.1 | 93347_Small Airway | 1.0 |
| 4-6 in IL-2 | | Epithelium_none | |
| 93567_primary Tr1_resting dy | 10.4 | 93348_Small Airway | 23.0 |
| 4-6 in IL-2 | | Epithelium_NFa (4 ng/ml) | |
| | | and IL1b (1 ng/ml) | |
| 93351_CD45RA CD4 | 6.4 | 92668_Coronery Artery | 4.1 |
| lymphocyte_anti-CD28/anti- | | SMC_resting | |
| CD3 | | | |
| 93352_CD45RO CD4 | 10.1 | 92669_Coronery Artery | 2.5 |
| lymphocyte_anti-CD28/anti- | | SMC_TNFa (4 ng/ml) and IL1b | |
| CD3 | | (1 ng/ml) | |
| 93251_CD8 | 6.3 | 93107_astrocytes_resting | 3.5 |
| Lymphocytes_anti-CD28/ | | | |
| anti-CD3 | | | |
| 93353_chronic CD8 | 6.9 | 93108_astrocytes_TNFa (4 | 3.7 |
| Lymphocytes 2ry_resting dy 4- | | ng/ml) and IL1b (1 ng/ml) | |
| 6 in IL-2 | | | |
| 93574_chronic CD8 | 6.6 | 92666_KU-812 | 3.2 |
| Lymphocytes 2ry_activated | | (Basophil)_resting | |
| CD3/CD28 | | | |
| 93354_CD4_none | 4.3 | 92667_KU-812 | 7.8 |
| | | (Basophil) _PMA/ionoycin | |
| 93252_Secondary | 8.5 | 93579_CCD1106 | 4.1 |
| Th1/Th2/Tr1_anti-CD95 CH11 | | (Keratinocytes)_none | |
| 93103_LAK_cells_resting | 21.2 | 93580_CCD1106 | 4.2 |
| | | (Keratinocytes)_TNFa and | |
| | | IFNg** | |
| 93788_LAK cells_IL-2 | 14.7 | 93791_Liver Cirrhosis | 2.2 |
| 93787_LAK cells_IL-2 + | 7.7 | 93792_Lupus Kidney | 2.1 |
| IL-12 | | | |
| 93789_LAK cells_IL-2 + IFN | 14.6 | 93577_NCI-H292 | 14.8 |
| gamma | | | |
| 93790_LAK cells_IL-2 + | 11.5 | 93358_NCI-H292_IL-4 | 14.2 |
| IL-18 | | | |
| 93104_LAK | 7.3 | 93360_NCI-H292_IL-9 | 16.2 |
| cells_PMA/ionomycin and IL- | | | |
| 18 | | | |
| 93578_NK Cells IL-2_resting | 11.3 | 93359_NCI-H292_IL-13 | 9.5 |
| 93109_Mixed Lymphocyte | 12.9 | 93357_NCI-H292_IFN gamma | 11.2 |
| Reaction_Two Way MLR | | | |
| 93110_Mixed Lymphocyte | 4.5 | 93777_HPAIEC_- | 5.6 |
| Reaction_Two Way MLR | | | |
| 93111_Mixed Lymphocyte | 3.4 | 93778_HPAEC_IL-1 beta/TNA | 11.1 |
| Reaction_Two Way MLR | | alpha | |
| 93112_Mononuclear Cells | 6.3 | 93254_Normal Human Lung | 3.9 |
| (PBMCs)_resting | | Fibroblast_none | |
| 93113_Mononuclear Cells | 22.8 | 93253_Normal Human Lung | 2.4 |
| (PBMCs)_PWM | | Fibroblast_TNFa (4 ng/ml) and | |
| | | IL-lb (1 ng/ml) | |
| 93114_Mononuclear Cells | 8.0 | 93257_Normal Human Lung | 6.8 |
| (PBMCs)_PHA-L | | Fibroblast_IL-4 | |
| 93249_Ramos (B cell) _none | 7.7 | 93256_Normal Human Lung | 3.6 |
| | | Fibroblast_IL-9 | |
| 93250_Ramos (B | 9.8 | 93255_Normal Human Lung | 5.9 |
| cell) ionomycin | | Fibroblast_IL-13 | |
| 93349_B lymphocytes_PWM | 16.2 | 93258_Normal Human Lung | 3.2 |
| | | Fibroblast_IFN gamma | |
| 93350_B lymphoytes_CD40L | 15.8 | 93106_Dermal Fibroblasts | 8.9 |
| and IL-4 | | CCD1070_resting | |
| 92665_EOL-1 | 6.9 | 93361_Dermal Fibroblasts | 100.0 |
| (Eosinophil)_dbcAMP | | CCD1070_TNF alpha 4 ng/ml | |
| differentiated | | | |
| 93248_EOL-1 | 18.3 | 93105_Dermal Fibroblasts | 8.0 |
| (Eosinophil)_dbcAMP/PMAion | | CCD1070_IL-1 beta 1 ng/ml | |
| omycin | | | |
| 93356_Dendritic Cells_none | 13.8 | 93772_dermal fibroblast_IFN | 5.4 |
| | | gamma | |
| 93355_Dendritic Cells_LPS | 10.4 | 93771_dermal fibroblast_IL-4 | 7.4 |
| 100 ng/ml | | | |
| 93775_Dendritic Cells_anti- | 12.7 | 93260_IBD Colitis 2 | 1.3 |
| CD40 | | | |
| 93774_Monocytes_resting | 42.6 | 93261_IBD Crohns | 0.5 |
| 93776_Monocytes_LPS 50 | 38.4 | 735010_Colon_normal | 5.2 |
| ng/ml | | | |
| 93581_Macrophages_resting | 24.5 | 735019_Lung_none | 7.6 |
| 93582_Macrophages_LPS 100 | 9.3 | 64028-1_Thymus_none | 8.6 |
| ng/ml | | | |
| 93098_HUVEC | 5.0 | 64030-1_Kidney_none | 15.8 |
| (Endothelial) _none | | | |
| 93099_HUVEC | 18.2 | | |
| (Endothelial) _starved | | | |

TABLE 28
Panel CNS_neurodegeneration_v1.0

| | Relative | | Relative |
|---|---|---|---|
| | Expression (%) | | Expression (%) |
| | tm7017t_{—} | | tm7017t_{—} |
| Tissue Name | ag2429_a1_s1 | Tissue Name | ag2429_a1_s1 |
| AD 1 Hippo | 0.0 | Control (Path) 3 Temporal Ctx | 0.0 |
| AD 2 Hippo | 0.0 | Control (Path) 4 Temporal Ctx | 0.0 |
| AD 3 Hippo | 0.0 | AD 1 Occipital Ctx | 0.0 |
| AD 4 Hippo | 0.0 | AD 2 Occipital Ctx (Missing) | 0.0 |
| AD 5 Hippo | 45.3 | AD 3 Occipital Ctx | 0.0 |
| AD 6 Hippo | 0.0 | AD 4 Occipital Ctx | 0.0 |
| Control 2 Hippo | 0.0 | AD 5 Occipital Ctx | 21.8 |
| Control 4 Hippo | 0.0 | AD 6 Occipital Ctx | 0.0 |
| Control (Path) 3 Hippo | 0.0 | Control 1 Occipital Ctx | 0.0 |
| AD 1 Temporal Ctx | 0.0 | Control 2 Occipital Ctx | 0.0 |
| AD 2 Temporal Ctx | 0.0 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital Ctx | 0.0 |
| AD 5 Inf Temporal Ctx | 100.0 | Control (Path) 2 Occipital Ctx | 0.0 |
| AD 5 Sup Temporal Ctx | 61.3 | Control (Path) 3 Occipital Ctx | 0.0 |
| AD 6 Inf Temporal Ctx | 0.0 | Control (Path) 4 Occipital Ctx | 0.0 |
| AD 6 Sup Temporal Ctx | 0.0 | Control 1 Parietal | 0.0 |
| Control 1 Temporal Ctx | 0.0 | Control 2 Parietal | 0.0 |
| Control 2 Temporal Ctx | 0.0 | Control 3 Parietal | 0.0 |
| Control 3 Temporal Ctx | 0.0 | Control (Path) 1 Parietal | 0.0 |
| Control 3 Temporal Ctx | 0.0 | Control (Path) 2 Parietal | 0.0 |
| Control (Path) 1 Temporal Ctx | 0.0 | Control (Path) 3 Parietal | 0.0 |
| Control (Path) 2 Temporal Ctx | 0.0 | Control (Path) 4 Parietal | 0.0 |

Summary Panel 1.2 Ag1504 The NOV4 gene is widely expressed among the tissue samples present in this panel. Highest expression of the NOV4 gene is detected in the bladder (CT=28.4). Thus, expression of this gene could be used to distinguish bladder tissue from other tissues.

Among metabolically relevant tissues, gene expression of NOV4 is detected at moderate levels in skeletal muscle, heart, adrenal gland, and adult liver. It is also expressed at low levels in fetal liver, thyroid, and the pituitary gland. The observation that the gene is expressed in adult heart tissue and skeletal muscle but not the corresponding fetal tissues suggests that expression of the NOV4 gene could be used to distinguish adult heart and skeletal muscle tissue from their fetal counterparts.

For tissues active in the central nervous system, the NOV4 gene is detected at moderate expression levels in the hippocampus, cerebellum, cerebral cortex, and at lower expression levels in the spinal cord, amygdala, and thalamus. This molecule is a homologue of cystatin-B, a non-caspase cysteine protease inhibitor. Loss-of-function mutations in the gene encoding cystatin B are associated with Unverricht-Lundborg disease, a severe neurological disorder resulting in seizures and ataxia. Cystatin-B has also been shown to be upregulated in response to severe seizure activity. Thus, upregulation of the NOV4 gene may have therapeutic value in the treatment of seizure disorders, specifically in preventing neuronal loss in response to seizures.

The NOV4 gene is expressed in adult lung (CT=33.7), but is undetectable in fetal lung. Thus, expression of the gene may be used to differentiate between adult and fetal lung tissue.

Summary Panel 1.3D Ag2429 Expression of the NOV4 gene is highest in a breast cancer cell line (CT=32.1)

Among metabolically relevant tissues, low but significant expression of the gene is detected in the adrenal and pituitary glands and fetal skeletal muscle. Expression of this gene is undetectable in adult skeletal muscle. The differential expression of the NOV4 gene in fetal skeletal muscle suggests that the protein product may be involved in the muscular growth or development of the fetus and hence may actually act in a regenerative capacity in the adult. Therefore, up-regulation of the activity of the NOV4 gene product in the adult, through the application of the actual protein product or by gene replacement therapy, may be useful in the treatment of muscular related diseases and may aid in the restoring of muscle mass or function in weak or dystrophic muscle.

In tissues active in the central nervous system, low levels of NOV4 gene expression are detected in the amygdala, hippocampus, thalamus, and the cerebral cortex. Please see panel 1.2 for discussion of the potential utility of this gene in the central nervous system.

The NOV4 gene is also expressed in adult lung (CT=34.5), but not in fetal lung tissue. This suggests that expression of the NOV4 gene could be used to distinguish between fetal and adult lung tissues.

Summary Panel 2D Ag1504/Ag2429 In two experiments using the probe and primer set Ag1504, and a third using the probe and primer set Ag2429, expression of the NOV4 gene is present in most of the tissue samples in this panel, with most significant expression in ovarian cancer, liver cancer and normal lung tissue. There is also significant expression in gastric cancer. Furthermore, the NOV4 gene appears to be overexpressed in ovarian cancer and gastric cancer when compared to their normal adjacent tissue. Therefore, expression of this gene could be used to distinguish ovarian, liver and gastric cancers from normal tissue and as a diagnostic marker for the presence of ovarian, liver and gastric cancers. In addition, therapeutic inhibition of the activity of the protein product, through the use of antibodies or small molecule drugs, may be effective in the treatment of ovarian, liver and gastric cancers.

Summary Panel 4D Ag2429 Expression of the NOV4 gene is ubiquitous at low levels throughout the samples in this panel. Highest expression is detected in dermal fibroblasts treated with the inflammatory cytokines TNF-a and IL-1b. This gene has homology to cystatin B, an inhibitor of cystein proteinases, whose presence has been shown to correlate with the degree of inflammation in different tissues. Therefore, therapeutic modulation of the expression of the gene NOV4 or the activity of its protein product, through the use of small molecule drugs, antibodies or protein therapeutics, may be beneficial in the treatment of inflammatory skin or lung diseases and other tissue inflammatory diseases such as rheumatoid arthritis.

Summary Panel CNS_neurogeneration_v1.0 Ag2429 Significant expression of the NOV4 gene is limited to the cerebral cortex and hippocampus of a single Alzheimer's patient.

References

1. Pennacchio L A, Bouley D M, Higgins K M, Scott M P, Noebels J L, Myers R M. (1998) Progressive ataxia, myoclonic epilepsy and cerebellar apoptosis in cystatin B-deficient mice. Nat Genet 20:251-8.

Loss-of-function mutations in the gene (CSTB) encoding human cystatin B, a widely expressed cysteine protease inhibitor, are responsible for a severe neurological disorder known as Unverricht-Lundborg disease or myoclonus epilepsy (EPM1). The primary cellular events and mechanisms underlying the disease are unknown. We found that mice lacking cystatin B develop myoclonic seizures and ataxia, similar to symptoms seen in the human disease. The principal cytopathology appears to be a loss of cerebellar granule cells, which frequently display condensed nuclei, fragmented DNA and other cellular changes characteristic of apoptosis. This mouse model of EPM1 provides evidence that cystatin B, a non-caspase cysteine protease inhibitor, has a role in preventing cerebellar apoptosis.

2. D'Amato E, Kokaia Z, Nanobashvili A, Reeben M, Lehesjoki A E, Saarma M, Lindvall O. (2000) Seizures induce widespread upregulation of cystatin B, the gene mutated in progressive myoclonus epilepsy, in rat forebrain neurons. Eur J Neurosci. 12:1687-95.

Loss of function mutations in the gene encoding the cysteine protease inhibitor, cystatin B (CSTB), are responsible for the primary defect in human progressive myoclonus epilepsy (EPM 1). CSTB inhibits the cathepsins B, H, L and S by tight reversible binding, but little is known regarding its localization and physiological function in the brain and the relation between the depletion of the CSTB protein and the clinical symptoms in EPM1. We have analysed the expression of mRNA and protein for CSTB in the adult rat brain using in situ hybridization and immunocytochemistry. In the control brains, the CSTB gene was differentially expressed with the highest levels in the hippocampal formation and reticular thalamic nucleus, and moderate levels in amygdala, thalamus, hypothalamus and cortical areas. Detectable levels of CSTB were found in virtually all forebrain neurons but not in glial cells. Following 40 rapidly recurring seizures evoked by hippocampal kindling stimulations, CSTB mRNA expression showed marked bilateral increases in the dentate granule cell layer, CA1 and CA4 pyramidal layers, amygdala, and piriform and parietal cortices. Maximum levels were detected at 6 or 24 h, and expression had reached control values at 1 week post-seizures. The changes of mRNA expression were accompanied by transient elevations (at 6-24 h) of CSTB protein in the same brain areas. These findings demonstrate that seizure activity leads to rapid and widespread increases of the synthesis of CSTB in forebrain neurons. We propose that the upregulation of CSTB following seizures may counteract apoptosis by binding cysteine proteases.

NOV5

Expression of gene NOV5 was assessed using the primer-probe sets Ag1558, Ag1507 and Ag1602, described in Tables 29, 30, and 31. Results from RTQ-PCR runs are shown in Tables 32, 33, 34, and 35.

TABLE 29

Probe Name Ag1558

| | | | | |
|---|---|---|---|---|
| | | | | Start |
| Primers | Sequences | TM | Length | Position |

| | | | | |
|---|---|---|---|---|
| Forward | 5′-CCCCTGATTTACACAGCTTTTA-3′ (SEQ ID NO: 60) | 58.3 | 22 | 1073 |
| Probe | TET-5′-ACAACAATGCCTTCAAGAGCCTCTTT-3′-TAMRA | 66.4 | 26 | 1104 |
| | (SEQ ID NO: 61) | | | |
| Reverse | 5′-CCCTGTGTTCATCTCTGCTTAG-3′ (SEQ ID NO: 62) | 59 | 22 | 1131 |

TABLE 30

+HC,1Probe Name Ag1507

| | | | | |
|---|---|---|---|---|
| | | | | Start |
| Primers | Sequences | TM | Length | Position |

| | | | | |
|---|---|---|---|---|
| Forward | 5′-CCCCTGATTTACACAGCTTTTA-3′ (SEQ ID NO: 63) | 58.3 | 22 | 1076 |
| Probe | TET-5′-ACAACAATGCCTTCAAGAGCCTCTTT-3′-TAMRA | 66.4 | 26 | 1107 |
| | (SEQ ID NO: 64) | | | |
| Reverse | 5′-CCCTGTGTTCATCTCTGCTTAG-3′ (SEQ ID NO:65) | 59 | 22 | 1134 |

TABLE 31

Probe Name Ag16O2

| | | | | |
|---|---|---|---|---|
| | | | | Start |
| Primers | Sequences | TM | Length | Position |

| | | | | |
|---|---|---|---|---|
| Forward | 5′-CCCCTGATTTACACAGCTTTTA-3′ (SEQ ID NO: 66) | 58.3 | 22 | 1065 |
| Probe | TET-5′-ACAACAATGCCTTCAAGAGCCTCTTT-3′-TAMRA | 66.4 | 26 | 1096 |
| | (SEQ ID NO: 67) | | | |
| Reverse | 5′-CCCTGTGTTCATCTCTGCTTAG-3′ (SEQ ID NO: 68) | 59 | 22 | 1123 |

TABLE 32
Panel 1.2

| | Relative | | Relative |
|---|---|---|---|
| | Expression (%) | | Expression (%) |
| | 1.2tm2155_{—} | | 1.2tm2155t_{—} |
| Tissue Name | ag1507 | Tissue Name | ag1507 |
| Endothelial cells | 7.5 | Renal ca. 786-0 | 0.9 |
| Heart (fetal) | 5.3 | Renal ca. A498 | 27.4 |
| Pancreas | 8.7 | Renal ca. RXF 393 | 2.3 |
| Pancreatic ca. CAPAN | 23.0 | Renal ca. ACHN | 15.5 |
| Adrenal Gland (new lot*) | 4.4 | Renal ca. UO-31 | 19.2 |
| Thyroid | 2.3 | Renal ca. TK-10 | 39.8 |
| Salivary gland | 12.9 | Liver | 5.8 |
| Pituitary gland | 0.0 | Liver (fetal) | 0.0 |
| Brain (fetal) | 0.0 | Liver ca. (hepatoblast) HepG2 | 27.2 |
| Brain (whole) | 4.0 | Lung | 0.0 |
| Brain (amygdala) | 20.3 | Lung (fetal) | 1.0 |
| Brain (cerebellum) | 3.2 | Lung ca. (small cell) LX-1 | 9.0 |
| Brain (hippocampus) | 13.0 | Lung ca. (small cell) NCI-H69 | 34.6 |
| Brain (thalamus) | 3.6 | Lung ca. (s.cell var.) SHP-77 | 2.0 |
| Cerebral Cortex | 16.6 | Lung ca. (large cell) NCI-H460 | 4.9 |
| Spinal cord | 0.0 | Lung ca. (non-sm. cell) A549 | 19.6 |
| CNS ca. (glio/astro) U87-MG | 10.6 | Lung ca. (non-s.cell) NCI-H23 | 25.7 |
| CNS ca. (glio/astro) U-118-MG | 3.8 | Lung ca (non-s.cell) HOP-62 | 35.8 |
| CNS ca. (astro) SW1783 | 2.0 | Lung ca. (non-s.d) NCI-H522 | 21.6 |
| CNS ca.* (neuro; met) SK-N- | 1.6 | Lung ca. (squam.) SW 900 | 21.2 |
| AS | | | |
| CNS ca. (astro) SF-539 | 4.7 | Lung ca. (squam.) NCI-H596 | 3.3 |
| CNS ca. (astro) SNB-75 | 2.1 | Mammary gland | 1.1 |
| CNS ca. (glio) SNB-19 | 16.4 | Breast ca.* (pl. effusion) | 2.0 |
| | | MCF-7 | |
| CNS ca. (glio) U251 | 9.2 | Breast ca.* (pl.ef) | 2.9 |
| | | MDA-MB-231 | |
| CNS ca. (glio) SF-295 | 3.2 | Breast ca.* (pl. effusion) T47D | 20.7 |
| Heart | 19.2 | Breast ca. BT-549 | 11.4 |
| Skeletal Muscle (new lot*) | 1.4 | Breast ca. MDA-N | 30.1 |
| Bone marrow | 0.9 | Ovary | 17.0 |
| Thymus | 0.0 | Ovarian ca. OVCAR-3 | 5.3 |
| Spleen | 3.9 | Ovarian ca. OVCAR-4 | 13.9 |
| Lymph node | 1.2 | Ovarian ca. OVCAR-5 | 100.0 |
| Colorectal | 6.0 | Ovarian ca. OVCAR-8 | 72.7 |
| Stomach | 0.9 | Ovarian ca. IGROV-1 | 49.3 |
| Small intestine | 6.0 | Ovarian ca.* (ascites) SK-OV-3 | 36.1 |
| Colon ca. SW480 | 2.3 | Uterus | 1.0 |
| Colon ca.* (SW480 met) SW620 | 0.0 | Placenta | 0.0 |
| Colon ca. HT29 | 14.6 | Prostate | 3.0 |
| Colon ca. HCT-116 | 13.5 | Prostate ca.* (bone met) PC-3 | 16.0 |
| Colon ca. CaCo-2 | 3.5 | Testis | 30.8 |
| 83219 CC Well to Mod Diff | 17.8 | Melanoma Hs688 (A) .T | 2.0 |
| (ODO3866) | | | |
| Colon ca. HCC-2998 | 35.1 | Melanoma* (met) Hs688 (B) .T | 7.3 |
| Gastric ca.* (liver met) NCI- | 14.6 | Melanoma UACC-62 | 6.0 |
| N87 | | | |
| Bladder | 38.4 | Melanoma M14 | 57.4 |
| Trachea | 0.0 | Melanoma LOX IMVI | 12.2 |
| Kidney | 28.5 | Melanoma* (met) SK-MEL-5 | 3.7 |
| Kidney (fetal) | 8.4 | | |

TABLE 33
Panel 2D

| | Relative | Relative |
|---|---|---|
| | Expression (%) | Expression (%) |
| | 2dtm4625t_{—} | 2dtm4116t_{—} |
| Tissue Name | ag1602 | ag1558 |
| Normal Colon GENPAK 061003 | 35.6 | 23.8 |
| 83219 CC Well to Mod Diff (ODO3866) | 47.3 | 19.5 |
| 83220 CC NAT (ODO3866) | 11.3 | 9.9 |
| 83221 CC Gr.2 rectosigmoid (ODO3868) | 27.2 | 8.2 |
| 83222 CC NAT (ODO3868) | 4.0 | 0.0 |
| 83235 CC Mod Diff (ODO3920) | 0.0 | 17.6 |
| 83236 CC NAT (ODO3920) | 9.0 | 16.4 |
| 83237 CC Gr.2 ascend colon (ODO3921) | 0.0 | 28.9 |
| 83238 CC NAT (ODO3921) | 27.9 | 17.9 |
| 83241 CC from Partial Hepatectomy (ODO4309) | 8.1 | 9.3 |
| 83242 Liver NAT (ODO4309) | 8.7 | 0.0 |
| 87472 Colon mets to lung (ODO4451-01) | 9.0 | 0.0 |
| 87473 Lung NAT (ODO4451-02) | 15.5 | 0.0 |
| Normal Prostate Clontech A + 6546-1 | 22.7 | 0.0 |
| 84140 Prostate Cancer (ODO44 10) | 0.0 | 8.4 |
| 84141 Prostate NAT (ODO4410) | 10.8 | 8.0 |
| 87073 Prostate Cancer (ODO4720-01) | 25.9 | 9.3 |
| 87074 Prostate NAT (ODO4720-02) | 25.7 | 33.7 |
| Normal Lung GENPAK 061010 | 100.0 | 60.3 |
| 83239 Lung Met to Muscle (ODO4286) | 27.2 | 7.1 |
| 83240 Muscle NAT (ODO4286) | 28.5 | 0.0 |
| 84136 Lung Malignant Cancer (ODO3126) | 11.5 | 9.8 |
| 84137 LungNAT (ODO3126) | 11.2 | 35.6 |
| 84871 Lung Cancer (ODO4404) | 10.1 | 4.7 |
| 84872 Lung NAT (ODO4404) | 0.0 | 8.9 |
| 84875 Lung Cancer (ODO4565) | 0.0 | 0.0 |
| 84876 Lung NAT (ODO4565) | 7.4 | 11.3 |
| 85950 Lung Cancer (ODO4237-01) | 0.0 | 8.1 |
| 85970 Lung NAT (ODO4237-02) | 17.7 | 7.6 |
| 83255 Ocular Mel Met to Liver (ODO4310) | 0.0 | 0.0 |
| 83256 Liver NAT (ODO4310) | 0.0 | 0.0 |
| 84139 Melanoma Mets to Lung (ODO4321) | 0.0 | 6.7 |
| 84138 Lung NAT (ODO4321) | 27.4 | 0.0 |
| Normal Kidney GENPAK 061008 | 9.5 | 36.1 |
| 83786 Kidney Ca, Nuclear grade 2 (ODO4338) | 0.0 | 0.0 |
| 83787 Kidney NAT (ODO4338) | 0.0 | 0.0 |
| 83788 Kidney Ca, Nuclear grade ½ (ODO4339) | 27.5 | 15.9 |
| 83789 Kidney NAT (ODO4339) | 28.5 | 8.6 |
| 83790 Kidney Ca, Clear cell type (ODO4340) | 16.0 | 14.0 |
| 83791 Kidney NAT (ODO4340) | 17.9 | 0.0 |
| 83792 Kidney Ca, Nuclear grade 3 (ODO4348) | 0.0 | 16.8 |
| 83793 Kidney NAT (ODO4348) | 9.0 | 29.3 |
| 87474 Kidney Cancer (ODO4622-01) | 0.0 | 0.0 |
| 87475 Kidney NAT (ODO4622-03) | 0.0 | 0.0 |
| 85973 Kidney Cancer (ODO4450-01) | 14.0 | 0.0 |
| 85974 Kidney NAT (ODO4450-03) | 0.0 | 5.5 |
| Kidney Cancer Clontech 8120607 | 0.0 | 0.0 |
| Kidney NAT Clontech 8120608 | 0.0 | 0.0 |
| Kidney Cancer Clontech 8120613 | 3.8 | 0.0 |
| Kidney NAT Clontech 8120614 | 0.0 | 0.0 |
| Kidney Cancer Clontech 9010320 | 14.2 | 0.0 |
| Kidney NAT Clontech 9010321 | 18.3 | 0.0 |
| Normal Uterus GEINPAK 061018 | 0.0 | 13.1 |
| Uterus Cancer GENPAK 064011 | 18.2 | 8.9 |
| Normal Thyroid Clontech A + 6570-1 | 0.0 | 0.0 |
| Thyroid Cancer GENPAK 064010 | 0.0 | 0.0 |
| Thyroid Cancer INVITROGEN A302152 | 5.0 | 0.0 |
| Thyroid NAT INVITROGEN A302153 | 18.7 | 30.4 |
| Normal Breast GENPAK 061019 | 0.0 | 21.9 |
| 84877 Breast Cancer (ODO4566) | 31.0 | 8.1 |
| 85975 Breast Cancer (ODO4590-01) | 7.7 | 24.7 |
| 85976 Breast Cancer Mets (ODO4590-03) | 10.9 | 20.3 |
| 87070 Breast Cancer Metastasis (ODO4655-05) | 40.9 | 11.7 |
| GENPAK Breast Cancer 064006 | 8.5 | 25.2 |
| Breast Cancer Res. Gen. 1024 | 0.0 | 8.5 |
| Breast Cancer Clontech 9100266 | 0.0 | 0.0 |
| Breast NAT Clontech 9100265 | 0.0 | 7.4 |
| Breast Cancer INVITROGEN A209073 | 9.0 | 25.3 |
| Breast NAT INVITROGEN A2090734 | 25.9 | 8.8 |
| Normal Liver GENPAK 061009 | 17.3 | 19.6 |
| Liver Cancer GENPAK 064003 | 13.6 | 16.6 |
| Liver Cancer Research Genetics RNA 1025 | 10.2 | 8.6 |
| Liver Cancer Research Genetics RNA 1026 | 0.0 | 9.5 |
| Paired Liver Cancer Tissue Research Genetics RNA 6004-T | 9.3 | 24.7 |
| Paired Liver Tissue Research Genetics RNA 6004-N | 0.0 | 9.3 |
| Paired Liver Cancer Tissue Research Genetics RNA 6005-T | 10.0 | 0.0 |
| Paired Liver Tissue Research Genetics RNA 6005-N | 0.0 | 0.0 |
| Normal Bladder GENPAK 061001 | 0.0 | 9.9 |
| Bladder Cancer Research Genetics RNA 1023 | 0.0 | 9.0 |
| Bladder Cancer INVITROGEN A302173 | 32.1 | 54.7 |
| 87071 Bladder Cancer (ODO4718-01) | 9.3 | 8.0 |
| 87072 Bladder Normal Adjacent (ODO4718-03) | 6.3 | 17.2 |
| Normal Ovary Res. Gen. | 8.5 | 0.0 |
| Ovarian Cancer GENPAK 064008 | 10.2 | 17.6 |
| 87492 Ovary Cancer ODO4768-07 | 27.0 | 9.4 |
| 87493 Ovary NAT ODO4768-08 | 0.0 | 0.0 |
| Normal Stomach GENPAK 061017 | 5.0 | 7.7 |
| Gastric Cancer Clontech 9060358 | 0.0 | 0.0 |
| NAT Stomach Clontech 9060359 | 0.0 | 8.5 |
| Gastric Cancer Clontech 9060395 | 3.9 | 4.9 |
| NAT Stomach Clontech 9060394 | 18.2 | 32.8 |
| Gastric Cancer Clontech 9060397 | 9.9 | 10.2 |
| NAT Stomach Clontech 9060396 | 0.0 | 0.0 |
| Gastric Cancer GENPAK 064005 | 50.7 | 100.0 |

TABLE 34
Panel 4D

| | Relative | Relative |
|---|---|---|
| | Expression (%) | Expression (%) |
| | 4dx4tm5019t_{—} | 4dtm4117t_{—} |
| Tissue Name | ag1507_b1 | ag1558 |
| 93768_Secondary Th1_anti-CD28/anti-CD3 | 48.8 | 29.5 |
| 93769_Secondary Th2_anti-CD28/anti-CD3 | 17.4 | 31.9 |
| 93770_Secondary Tr1_anti-CD28/anti-CD3 | 10.7 | 18.0 |
| 93573_Secondary Th1_resting day 4-6 in IL-2 | 0.0 | 0.0 |
| 93572_Secondary Th2_resting day 4-6 in IL-2 | 8.3 | 7.5 |
| 93571_Secondary Tr1_resting day 4-6 in IL-2 | 0.0 | 7.3 |
| 93568_primary Th1_anti-CD28/anti-CD3 | 57.6 | 17.7 |
| 93569_primary Th2_anti-CD28/anti-CD3 | 8.0 | 42.0 |
| 93570_primary Tr1_anti-CD28/anti-CD3 | 27.2 | 43.2 |
| 93565_primary Th1_resting dy 4-6 in IL-2 | 56.1 | 34.6 |
| 93560_primary Th2_resting dy 4-6 in IL-2 | 23.2 | 20.0 |
| 93567_primary Tr1_resting dy 4-6 in IL-2 | 9.0 | 15.8 |
| 93351_CD45RA CD4 lymphocyte_anti-CD28/anti-CD3 | 7.1 | 48.3 |
| 93352_CD45RO CD4 lymphocyte_anti-CD28/anti-CD3 | 34.5 | 31.0 |
| 93251_CD8 Lymphocytes_anti-CD28/anti-CD317.3 | 16.3 | |
| 93353_chronic CD8 Lymphocytes 2ry_resting dy 4-6 | 8.3 | 32.5 |
| in IL-2 | | |
| 93574_chronic CD8 Lymphocytes 2ry_activated | 10.4 | 12.3 |
| CD3/CD28 | | |
| 93354_CD4 none | 13.9 | 15.8 |
| 93252_Secondary Th1/Th2ITrl anti-CD95 CH11 | 15.6 | 0.0 |
| 93103_LAK cells_resting | 17.1 | 54.7 |
| 93788_LAK cells_IL-2 | 30.5 | 13.4 |
| 93787_LAK cells_IL-2 + IL-12 | 25.1 | 8.0 |
| 93789_LAK cells_IL-2 + IFN gamma | 51.0 | 30.4 |
| 93790_LAK cells_IL-2 + IL-18 | 12.4 | 84.1 |
| 93104_LAK cells PMA/ionomycin and IL-18 | 16.7 | 24.8 |
| 93578_NK Cells IL-2_resting | 37.0 | 32.3 |
| 93109_Mixed Lymphocyte Reaction_Two Way MLR | 8.1 | 48.6 |
| 93110_Mixed Lymphocyte Reaction_Two Way MLR | 7.5 | 15.7 |
| 93111_Mixed Lymphocyte Reaction_Two Way MLR | 7.4 | 0.0 |
| 93112_Mononuclear Cells (PBMCs)_resting | 0.0 | 7.2 |
| 93113_Mononuclear Cells (PBMCs)_PWM | 100.0 | 64.2 |
| 93114_Mononuclear Cells (PBMCs)_PHA-L | 71.0 | 23.8 |
| 93249_Ramos (B cell)_none | 0.0 | 8.1 |
| 93250_Ramos (B cell)_ionomycin | 42.1 | 36.9 |
| 93349_B lymphocytes_PWM | 12.7 | 69.3 |
| 93350_B lymphoytes_CD40L and IL-4 | 45.9 | 45.1 |
| 92665_EOL-1 (Eosinophil)_dbcAMP differentiated | 9.1 | 3.2 |
| 93248_EOL-1 (Eosinophil)_dbcAMP/PMAionomycin | 6.6 | 30.4 |
| 93356_Dendritic Cells_none | 51.8 | 26.8 |
| 93355_Dendritic Cells_LPS 100 ng/ml | 15.3 | 0.0 |
| 93775_Dendritic Cells_anti-CD40 | 20.8 | 0.0 |
| 93774_Monocytes_resting | 7.4 | 0.0 |
| 93776_Monocytes_LPS 50 ng/ml | 47.8 | 37.1 |
| 93581_Macrophages_resting | 22.2 | 32.3 |
| 93582_Macrophages_LPS 100 ng/ml | 0.0 | 16.3 |
| 93098_HUVEC (Endothelial)_none | 0.0 | 0.0 |
| 93099_HUVEC (Endothelial)_starved | 10.9 | 30.6 |
| 93100_HUVEC (Endothelial)_IL-lb | 0.0 | 0.0 |
| 93779_HUVEC (Endothelial)_IFN gamma | 0.0 | 8.5 |
| 93102_HUVEC (Endothelial)_TNF alpha + IFN gamma | 0.0 | 18.2 |
| 93101_HUVEC (Endothelial)_TNF alpha + IL4 | 0.0 | 0.0 |
| 93781_HUVEC (Endothelial)_IL-11 | 0.0 | 0.0 |
| 93583_Lung Microvascular Endothelial | 5.1 | 4.2 |
| Cells_none | | |
| 93584_Lung Microvascular Endothelial | 0.0 | 7.6 |
| Cells_TNFa (4 ng/ml) | | |
| and IL1b (1 ng/ml) | | |
| 92662_Microvascular Dermal endothelium_none | 19.2 | 6.4 |
| 92663_Microsvasular Dermal endothelium_TNFa | 9.6 | 0.0 |
| (4 ng/ml) and IL1b (1 ng/ml) | | |
| 93773_Bronchial epithelium_TNFa (4 ng/ml) and IL1b (1 | 0.0 | 0.0 |
| ng/ml) ** | | |
| 93347_Small Airway Epithelium_none | 0.0 | 7.6 |
| 93348_Small Airway Epithelium_TNFa (4 ng/ml) | 80.6 | 49.7 |
| and IL1b (1 ng/ml) | | |
| 92668_Coronery Artery SMC_resting | 10.3 | 0.0 |
| 92669_Coronery Artery SMC_TNFa (4 ng/ml) | 7.3 | 7.9 |
| and IL1b (1 ng/ml) | | |
| 93107_astrocytes_resting | 0.0 | 0.0 |
| 93108_astrocytes_TNFa (4 ng/ml) and IL1b (1 ng/ml) | 0.0 | 8.2 |
| 92666_KU-812 (Basophil)_resting | 0.0 | 7.6 |
| 92667_KU-812 (Basophil)_PMA/ionoycin | 20.9 | 7.3 |
| 93579_CCD1106 (Keratmocytes)_none | 4.2 | 7.3 |
| 93580_CCD1106 (Keratinocytes)_TNFa and IFNg** | 0.0 | 0.0 |
| 93791_Liver Cirrhosis | 18.8 | 94.6 |
| 93792_Lupus Kidney | 0.0 | 0.0 |
| 93577_NCI-H292 | 14.5 | 14.6 |
| 93358_NCI-H292_IL-4 | 16.4 | 23.5 |
| 93360_NCI-H292_IL-9 | 28.0 | 7.3 |
| 93359_NCI-H292_IL-13 | 18.9 | 23.0 |
| 93357_NCI-H292_IFN gamma | 13.3 | 8.0 |
| 93777_HPAEC_- | 0.0 | 10.4 |
| 93778_HPAEC_IL-1 beta/TNA alpha | 18.9 | 0.0 |
| 93254_Normal Human Lung Fibroblast_none | 0.0 | 0.0 |
| 93253_Normal Human Lung Fibroblast_TNFa (4 ng/ml) | 8.0 | 0.0 |
| and IL-lb (1 ng/ml) | | |
| 93257_Normal Human Lung Fibroblast_IL-4 | 8.9 | 7.8 |
| 93256_Normal Human Lung Fibroblast_IL-9 | 7.7 | 16.3 |
| 93255_Normal Human Lung Fibroblast_IL-13 | 15.2 | 0.0 |
| 93258_Normal Human Lung Fibroblast_IFN gamma | 10.4 | 7.4 |
| 93106_Dermal Fibroblasts CCD1070_resting | 0.0 | 26.1 |
| 93361_Dermal Fibroblasts CCD1070_TNF alpha 4 ng/ml | 65.6 | 100.9 |
| 93105_Dermal Fibroblasts CCD1070_IL-1 beta 1 ng/ml | 14.7 | 31.0 |
| 93772_dermal fibroblast_IFN gamma | 0.0 | 9.6 |
| 93771_dermal fibroblast_IL-4 | 39.8 | 0.0 |
| 93260_IBD Colitis 2 | 8.0 | 8.1 |
| 93261_IBD Crohns | 8.2 | 14.7 |
| 735010_Colon_normal | 30.5 | 48.3 |
| 735019_Lung_none | 14.5 | 11.7 |
| 64028-1_Thymus_none | 22.1 | 10.1 |
| 64030-1_Kidney_none | 0.0 | 0.0 |

TABLE 35
Panel CNS_neurodegeneration_v1.0

| | Relative | | Relative |
|---|---|---|---|
| | Expression (%) | | Expression (%) |
| | tm6962t_{—} | | tm6962t_{—} |
| Tissue Name | ag1558_a2_s2 | Tissue Name | ag1558_a2_s2 |
| AD 1 Hippo | 10.8 | Control Path (3) Temporal Ctx | 23.5 |
| AD 2 Hippo | 100.0 | Control (Path) 4 Temporal Ctx | 46.5 |
| AD 3 Hippo | 0.0 | AD 1 Occipital Ctx | 0.0 |
| AD 4 Hippo | 0.0 | AD 2 Occipital Ctx (Missing) | 0.0 |
| AD 5 hippo | 0.0 | AD 3 Occipital Ctx | 0.0 |
| AD 6 Hippo | 35.3 | AD 4 Occipital Ctx | 0.0 |
| Control 2 Hippo | 0.0 | AD 5 Occipital Ctx | 30.7 |
| Control 4 Hippo | 0.0 | AD 6 Occipital Ctx | 0.0 |
| Control (Path) 3 Hippo | 11.4 | Control 1 Occipital Ctx | 0.0 |
| AD 1 Temporal Ctx | 0.0 | Control 2 Occipital Ctx | 0.0 |
| AD 2 Temporal Ctx | 63.6 | Control 3 Occipital Ctx | 0.0 |
| AD 3 Temporal Ctx | 0.0 | Control 4 Occipital Ctx | 0.0 |
| AD 4 Temporal Ctx | 0.0 | Control (Path) 1 Occipital Ctx | 0.0 |
| AD 5 Inf Temporal Ctx | 74.2 | Control (Path) 2 Occipital Ctx | 0.0 |
| AD 5 Sup Temporal Ctx | 33.9 | Control (Path) 3 Occipital Ctx | 0.0 |
| AD 6 Inf Temporal Ctx | 5.8 | Control (Path) 4 Occipital Ctx | 0.0 |
| AD 6 Sup Temporal Ctx | 72.2 | Control 1 Parietal | 0.0 |
| Control 1 Temporal Ctx | 52.1 | Control 2 Parietal | 0.0 |
| Control 2 Temporal Ctx | 35.9 | Control 3 Parietal | 26.7 |
| Control 3 Temporal Ctx | 18.8 | Control (Path) 1 Parietal | 0.0 |
| Control 4 Temporal Ctx | 0.0 | Control (Path) 2 Parietal | 33.6 |
| Control (Path) 1 Temporal Ctx | 0.0 | Control (Path) 3 Parietal | 0.0 |
| Control (Path) 2 Temporal Ctx | 18.3 | Control (Path) 4 Parietal | 30.2 |

Panel 1.2 Summary Ag1507 Low but significant expression of the GMAC009404_A gene is detected in ovarian cancer cell lines (CT=32.5). In general, there appears to be expression of this gene in cancer cell lines rather than in normal tissues, with low but significant expression also detectable in melanoma, breast cancer, lung cancer, and renal cancer cell lines. Thus, expression of the GMAC009404_A gene could be used to distinguish samples derived from melanoma, breast, lung, renal and colon cancers from other tissues. Furthermore, therapeutic inhibition of the GMAC009404_A gene or its protein product, throught the use of antibodies, small molecule or protein drugs, may be effective in the treatment of the aforementioned cancers.

Among metabolically relevant tissues, there is low but significant expression of the GMAC009404_A gene in adult heart tissue (CT=34.9), but not in fetal heart tissue. This result suggests that GMAC009404_A gene expression could be used as a marker to distinguish between fetal and adult heart tissue.

Panel 1.3D Summary Ag 1507/Ag1558/Ag1602 Expression of this gene in panel 1.3D is low/undetectable (Ct values>35) in all samples (data not shown).

Panel 2D Summary Ag1558 Significant expression of the GMAC009404_A gene is detected in a gastric cancer tissue sample (CT=34.7). Thus, expression of the gene could be used to distinguish between gastric cancer and normal tissue. Ag1602 Significant expression of the GMAC009404_A gene is detected in a tissue sample from normal lung (CT=34.2). The GMAC009404_A gene could therefore be used to distinguish between normal lung tissue and other tissues. Ag1507 Expression of the GMAC009404_A gene with this probe and primer set is low/undetectable (Ct values>35) in all samples (data not shown).

Panel 4D Summary Ag1507 Expression of the GMAC009404_A gene is limited to a few samples, with highest expression detected in activated B cells. This suggests that the GMAC009404_A gene product may play a role in diseases that have B cell involvement, such as rheumatoid arthritis, systemic lupus erythematosus, delayed type hypersensitivity and inflammatory bowel disease. Thus, therapeutic modulation of the GMAC009404_A gene or its protein product may be effective in the treatment of any of these diseases. Ag1558 Highest expression of the GMAC009404_A gene is observed in dermal fibroblasts treated with TNF alpha (CT=34.4). Ag1602 Expression of the GMAC009404_A gene with this probe and primer set is low/undetectable (Ct values>35) in all samples (data not shown).

Panel CNS_neurodegeneration_v1.0 Ag1558 Expression of the GMAC009404_A gene is highest in the hippocampus of an Alzheimer's patient. Ag1602 Expression of the GMAC009404_A gene with this probe and primer set is low/undetectable (Ct values>35) in all samples (data not shown).

References

1. Baguley B C, Cole G, Thomsen L L, Li Z. (1993) Serotonin involvement in the antitumour and host effects of flavone-8-acetic acid and 5,6-dimethylxanthenone-4-acetic acid. Cancer Chemother Pharmacol. 33:77-81.

The relationship of serotonin (5-HT) receptors to the action of the experimental antitumour drugs flavone-8-acetic acid (FAA) and 5,6-dimethylxanthenone-4-acetic acid (5,6-MeXAA) was studied. Both FAA and 5,6-MeXAA are known to induce the synthesis of tumour necrosis factor-alpha (TNF) and to stimulate nitric oxide synthesis in vivo, as measured by elevation of plasma nitrate. Serotonin potentiated the effect of a subtherapeutic dose of 5,6-MeXAA (20 mg/kg) as measured both by plasma nitrate increase and by growth delay of s.c. implanted colon 38 tumours. On the other hand, administration of the serotonin 5-hydroxytryptamine-2 (5-HT2) antagonist cyproheptadine (20 mg/kg) inhibited both the plasma nitrate response and, to a lesser extent, the induction of tumour haemorrhagic necrosis by 5,6-MeXAA, FAA and TNF. Reduction of circulating plasma serotonin by pre-treatment with p-chlorophenylalanine and reserpine reduced the plasma nitrate response, but not the tumour necrosis response, to 5,6-MeXAA (30 mg/kg). It is suggested that serotonin is necessary for the induction of nitric oxide synthases and acts, either directly or indirectly, in concert with TNF. Serotonin agonists may have utility in increasing nitric oxide synthesis in response to TNF or to agents that induce TNF as part of their antitumour action.

2. Zhao L, Kestell P, Philpott M, Ching L M, Zhuang L, Baguley B C. (2001) Effects of the serotonin receptor antagonist cyproheptadine on the activity and pharmacokinetics of 5,6-dimethylxanthenone-4-acetic acid (DMXAA). Cancer Chemother Pharmacol. 47:491-7.

BACKGROUND: DMXAA (5,6-dimethylxanthenone-4-acetic acid) is a new drug synthesized in this laboratory and currently in phase I clinical trial. In mice it acts as an antivascular drug, selectively inhibiting tumour blood flow and inducing tumour haemorrhagic necrosis with resultant tumour regression. It also induces the synthesis of tumour necrosis factor (TNF), nitric oxide and serotonin. Cyproheptadine, a type 2 serotonin receptor antagonist, is known to reduce the degree of tumour necrosis-induced TNF in mice. We investigated the pharmacological interaction between a suboptimal dose of DMXAA (20 mg/kg) and cyproheptadine (20 mg/ kg) using mice with Colon 38 tumours that are sensitive to DMXAA. METHODS: Mice with or without tumours were treated with DMXAA and/or cyproheptadine. Concentrations of plasma and tissue DMXAA and the serotonin metabolite 5-hydroxyindoleacetic acid were measured by high performance liquid chromatography. TNF concentrations were measured by ELISA. RESULTS: While DMXAA alone (20 mg/kg) showed little or no antitumour activity, coadministration with cyproheptadine was curative in four of five mice. DMXAA half-lives in plasma and tumour tissue were increased 5.1- and 5.6-fold, respectively, and the appearance of DMXAA glucuronides in bile was almost completely inhibited for up to 4 h. Serum TNF was low and unchanged by cyproheptadine, and plasma concentrations of the serotonin metabolite 5-hydroxyindoleacetic acid were also not substantially changed. CONCLUSION: The augmentation by cyproheptadine of the induction of tumour response to DMXAA reflects a pharmacological interaction, leading to increased plasma and tumour half-lives, and to reduced excretion. However, serum TNF concentrations were not increased, suggesting that the increased anti-tumour effects are mediated by an increased local tumour response, arising from the extended tumour DMXAA concentrations.

NOV6

Expression of gene NOV6was assessed using the primer-probe set Ag1584 described in Table 36. Results from RTQ-PCR runs are shown in Tables 37, 38, and 39.

TABLE 36

Probe Name Ag1584

| | | | | |
|---|---|---|---|---|
| | | | | Start |
| Primers | Sequences | TM | Length | Position |

| | | | | |
|---|---|---|---|---|
| Forward | 5′-GTAAGCGGCCACTCATCTTTAT-3′ (SEQ ID NO: 69) | 59.7 | 22 | 410 |
| Probe | FAM-5′-CAGCACAGTGCTCGTGTACACAAGCT-3′-TAMRA | 68.9 | 26 | 447 |
| | (SEQ ID NO: 70) | | | |
| Reverse | 5′-GCAGGCACTTTGTTCTTGTATC-3′ (SEQ ID NO: 71) | 58.9 | 22 | 476 |

TABLE 37
Panel 1.3D

| | Relative | | Relative |
|---|---|---|---|
| | Expression (%) | | Expression (%) |
| | 1.3dx4tm5587f_{—} | | 1.3dx4tm5587f_{—} |
| Tissue Name | ag1584_a2 | Tissue Name | ag1584_a2 |
| Liver adenocarcinoma | 19.4 | Kidney (fetal) | 0.0 |
| Pancreas | 0.0 | Renal ca. 786-0 | 0.0 |
| Pancreatic ca. CAPAN 2 | 15.6 | Renal ca. A498 | 19.4 |
| Adrenal gland | 9.4 | Renal ca. RXF 393 | 15.3 |
| Thyroid | 0.0 | Renal ca. ACHN | 0.0 |
| Salivary gland | 0.0 | Renal ca. UO-31 | 0.0 |
| Pituitary gland | 0.0 | Renal ca. TK-10 | 0.0 |
| Brain (fetal) | 0.0 | Liver | 0.0 |
| Brain (whole) | 9.5 | Liver (fetal) | 0.0 |
| Brain (amygdala) | 17.1 | Liver ca. (hepatoblast) HepG2 | 0.0 |
| Brain (cerebellum) | 9.6 | Lung | 0.0 |
| Brain (hippocampus) | 0.0 | Lung (fetal) | 9.7 |
| Brain (substantia nigra) | 32.4 | Lung ca. (small cell) LX-1 | 0.0 |
| Brain (thalamus) | 10.1 | Lung ca. (small cell) NCI-1169 | 0.0 |
| Cerebral Cortex | 19.0 | Lung ca. (s.cell var.) SHP-77 | 0.0 |
| Spinal cord | 0.0 | Lung ca. (large cell) NCI-H460 | 0.0 |
| CNS ca. (glio/astro) U87-MG | 36.6 | Lung ca. (non-sm. cell) A549 | 6.8 |
| CNS ca. (glio/astro) U-118-MG | 0.0 | Lung ca. (non-s.cell) NCI-H23 | 22.4 |
| CNS ca. (astro) SWl 783 | 0.0 | Lung ca (non-s.cell) HOP-62 | 7.7 |
| CNS ca.* (neuro; met) SK-N- | 0.0 | Lung ca. (non-s.d) NCI-H522 | 0.0 |
| AS | | | |
| CNS ca. (astro) SF-539 | 7.5 | Lung ca. (squam.) SW 900 | 0.0 |
| CNS ca. (astro) SNB-75 | 6.6 | Lung ca. (squam.) NCI-H596 | 0.0 |
| CNS ca. (glio) SNB-19 | 0.0 | Mammary gland | 21.8 |
| CNS ca. (glio) U251 | 0.0 | Breast ca.* (pl. effusion) MCF-7 | 0.0 |
| CNS ca. (glio) SF-295 | 0.0 | Breast ca.* (pl. ef) | 0.0 |
| | | MDA-MB-231 | |
| Heart (fetal) | 0.0 | Breast ca.* (pl. effusion) T47D | 40.2 |
| Heart | 0.0 | Breast ca. BT-549 | 0.0 |
| Fetal Skeletal | 0.0 | Breast ca. MDA-N | 34.0 |
| Skeletal muscle | 0.0 | Ovary | 5.3 |
| Bone marrow | 100.0 | Ovarian ca. OVCAR-3 | 20.9 |
| Thymus | 0.0 | Ovanan ca. OVCAR-4 | 75.1 |
| Spleen | 56.3 | Ovarian ca. OVCAR-5 | 15.0 |
| Lymph node | 0.0 | Ovarian ca. OVCAR-8 | 0.0 |
| Colorectal | 7.5 | Ovarian ca. IGROV-1 | 7.4 |
| Stomach | 0.0 | Ovarian ca.* (ascites) SK-OV-3 | 0.0 |
| Small intestine | 0.0 | Uterus | 0.0 |
| Colon ca. SW480 | 6.9 | Placenta | 0.0 |
| Colon ca.* (SW480 met) SW620 | 11.7 | Prostate | 0.0 |
| Colon ca. HT29 | 4.5 | Prostate ca.* (bone met) PC-3 | 0.0 |
| Colon ca. HCT-116 | 19.2 | Testis | 28.4 |
| Colon ca. CaCo-2 | 0.0 | Melanoma Hs688 (A) .T | 0.0 |
| 83219 CC Well to Mod Diff | 0.0 | Melanoma* (met) Hs688 (B) .T | 6.9 |
| (ODO3866) | | | |
| Colon ca. HCC-2998 | 24.7 | Melanoma UACC-62 | 0.0 |
| Gastric ca.* (liver met) NCI-87 | 7.2 | Melanoma M14 | 0.0 |
| Bladder | 0.0 | Melanoma LOX IMVI | 0.0 |
| Trachea | 0.0 | Melanoma* (met) SK-MEL-5 | 6.3 |
| Kidney | 11.1 | Adipose | 13.5 |

TABLE 38
Panel 2.2

| | Relative | | Relative |
|---|---|---|---|
| | Expression (%) | | Expression (%) |
| | 2.2x4tm6339f_{—} | | 2.2x4tm6339f_{—} |
| Tissue Name | ag1584_a2 | Tissue Name | ag1584_a2 |
| Normal Colon GENPAK | 0.0 | 83793 Kidney NAT (ODO4348) | 76.3 |
| 061003 | | | |
| 97759 Colon cancer (ODO6064) | 100.0 | 98938 Kidney malignant cancer | 0.0 |
| | | (ODO6204B) | |
| 97760 Colon cancer NAT | 27.6 | 98939 Kidney normal adjacent | 0.0 |
| (ODO6064) | | tissue (ODO6204E) | |
| 97778 Colon cancer (ODO6159) | 0.0 | 85973 Kidney Cancer | 11.4 |
| | | (ODO4450-01) | |
| 97779 Colon cancer NAT | 0.0 | 85974 Kidney NAT (ODO4450- | 19.0 |
| (ODO6159) | | 03) | |
| 98861 Colon cancer (ODO6297- | 0.0 | Kidney Cancer Clontech | 0.0 |
| 04) | | 8120613 | |
| 98862 Colon cancer NAT | 14.2 | Kidney NAT Clontech 8120614 | 0.0 |
| (ODO6297-015) | | | |
| 83237 CC Gr.2 ascend colon | 0.0 | Kidney Cancer Clontech | 12.9 |
| (ODO3921) | | 9010320 | |
| 83238 CC NAT (ODO3921) | 0.0 | Kidney NAT Clontech 9010321 | 18.8 |
| 97766 Colon cancer metastasis | 0.0 | Kidney Cancer Clontech | 0.0 |
| (ODO6104) | | 8120607 | |
| 97767 Lung NAT (ODO6104) | 0.0 | Kidney NAT Clontech 8120608 | 14.2 |
| 87472 Colon mets to lung | 8.7 | Normal Uterus GENPAK | 11.8 |
| (ODO4451-01) | | 061018 | |
| 87473 Lung NAT (ODO4451- | 67.1 | Uterus Cancer GENPAK | 0.0 |
| 02) | | 064011 | |
| Normal Prostate Clontech A + | 0.0 | Normal Thyroid Clontech A + | 0.0 |
| 6546-1 (8090438) | | 6570-1 (7080817) | |
| 84140 Prostate Cancer | 40.2 | Thyroid Cancer GENPAK | 0.0 |
| (ODO4410) | | 064010 | |
| 84141 Prostate NAT | 0.0 | Thyroid Cancer INVITROGEN | 10.0 |
| (ODO4410) | | A302152 | |
| Normal Ovary Res. Gen. | 16.4 | Thyroid NAT INVITROGEN | 0.0 |
| | | A302153 | |
| 98863 Ovarian cancer | 70.1 | Normal Breast GENPAK | 17.8 |
| (ODO6283-03) | | 061019 | |
| 98865 Ovarian cancer | 18.4 | 84877 Breast Cancer | 0.0 |
| NAT/fallopian tube (ODO6283- | | (ODO4566) | |
| 07) | | | |
| Ovarian Cancer GENPAK | 30.5 | Breast Cancer Res. Gen. 1024 | 10.4 |
| 064008 | | | |
| 97773 Ovarian cancer | 0.0 | 85975 Breast Cancer | 0.0 |
| (ODO6145) | | (ODO4590-01) | |
| 97775 Ovarian cancer NAT | 0.0 | 85976 Breast Cancer Mets | 0.0 |
| (ODO6145) | | (ODO4590-03) | |
| 98853 Ovarian cancer | 43.4 | 87070 Breast Cancer Metastasis | 0.0 |
| (ODO6455-03) | | (ODO4655-05) | |
| 98854 Ovarian NAT | 9.7 | GENPAK Breast Cancer | 63.8 |
| (ODO6455-07) Fallopian tube | | 064006 | |
| Normal Lung GENPAK 061010 | 0.0 | Breast Cancer Clontech | 0.0 |
| | | 9100266 | |
| 92337 Invasive poor diff. lung | 0.0 | Breast NAT Clontech 9100265 | 25.8 |
| adeno (ODO4945-01 | | | |
| 92338 Lung NAT (ODO4945- | 0.0 | Breast Cancer INVITROGEN | 0.0 |
| 03) | | A209073 | |
| 84136 Lung Malignant Cancer | 63.2 | Breast NAT INVITROGEN | 0.0 |
| (ODO3126) | | A2090734 | |
| 84137 Lung NAT (ODO3126) | 0.0 | 97763 Breast cancer | 49.7 |
| | | (ODO6083) | |
| 90372 Lung Cancer | 0.0 | 97764 Breast cancer node | 15.3 |
| (ODO5014A) | | metastasis (ODO6083) | |
| 90373 Lung NAT (ODO5014B) | 32.5 | Normal Liver GENPAK | 0.0 |
| | | 061009 | |
| 97761 Lung cancer (ODO6081) | 25.6 | Liver Cancer Research Genetics | 23.0 |
| | | RNA 1026 | |
| 97762 Lung cancer NAT | 16.2 | Liver Cancer Research Genetics | 0.0 |
| (ODO6081) | | RNA 1025 | |
| 85950 Lung Cancer (ODO4237- | 18.4 | Paired Liver Cancer Tissue | 0.0 |
| 01) | | Research Genetics RNA 6004- | |
| | | T | |
| 85970 Lung NAT (ODO4237- | 13.1 | Paired Liver Tissue Research | 0.0 |
| 02) | | Genetics RNA 6004-N | |
| 83255 Ocular Mel Met to Liver | 0.0 | Paired Liver Cancer Tissue | 32.5 |
| (ODO4310) | | Research Genetics RNA 6005-T | |
| 83256 Liver NAT (ODO4310) | 0.0 | Paired Liver Tissue Research | 0.0 |
| | | Genetics RNA 6005-N | |
| 84139 Melanoma Mets to Lung | 0.0 | Liver Cancer GENPAK 064003 | 22.4 |
| (ODO4321) | | | |
| 84138 Lung NAT (ODO4321) | 0.0 | Normal Bladder GENPAK | 0.0 |
| | | 061001 | |
| Normal Kidney GENPAK | 0.0 | Bladder Cancer Research | 12.7 |
| 061008 | | Genetics RNA 1023 | |
| 83786 Kidney Ca, Nuclear | 0.0 | Bladder Cancer INVITROGEN | 0.0 |
| grade 2 (ODO4338) | | A302173 | |
| 83787 Kidney NAT (ODO4338) | 0.0 | Normal Stomach GENPAK | 0.0 |
| | | 061017 | |
| 83788 Kidney Ca Nuclear grade | 0.0 | Gastric Cancer Clontech | 0.0 |
| ½ ODO4339 | | 9060397 | |
| 83789 Kidney NAT (ODO4339) | 0.0 | NAT Stomach Clontech | 0.0 |
| | | 9060396 | |
| 83790 Kidney Ca, Clear cell | 0.0 | Gastric Cancer Clontech | 0.0 |
| type (ODO4340) | | 9060395 | |
| 83791 Kidney NAT (ODO4340) | 19.3 | NAT Stomach Clontech | 29.4 |
| | | 9060394 | |
| 83792 Kidney Ca, Nuclear | 0.0 | Gastric Cancer GENPAK | 0.0 |
| grade 3 (ODO4348) | | 064005 | |

TABLE 39
Panel 4D

| | Relative |
|---|---|
| | Expression (%) |
| | 4dx4tm5535f_ |
| Tissue Name | ag1584_b2 |
| 93768_Secondary Th1_anti- | 0.0 |
| CD28/anti-CD3 | |
| 93769_Secondary Th2_anti- | 0.0 |
| CD28/anti-CD3 | |
| 93770_Secondary Tr1_anti- | 1.2 |
| CD28/anti-CD3 | |
| 93573_Secondary Th1_resting | 1.4 |
| day 4-6 in IL-2 | |
| 93572_Secondary Th2_resting | 0.0 |
| day 4-6 in IL-2 | |
| 93571_Secondary Tr1_resting | 0.0 |
| day 4-6 in IL-2 | |
| 93568_primary Th1_anti- | 0.0 |
| CD28/anti-CD3 | |
| 93569_primary Th2_anti- | 0.0 |
| CD28/anti-CD3 | |
| 93570_primary Tr1_anti- | 0.0 |
| CD28/anti-CD3 | |
| 93565_primary Th1_resting dy | 3.2 |
| 4-6 in IL-2 | |
| 93566_primary Th2_resting dy | 0.0 |
| 4-6 in IL-2 | |
| 93567_primary Tr1_resting dy | 0.0 |
| 4-6 in IL-2 | |
| 93351_CD45R CD4 | 0.0 |
| lymphocyte_anti-CD28/anti- | |
| CD3 | |
| 93352_CD45RO CD4 | 0.0 |
| lymphocyte_anti-CD28/anti- | |
| CD3 | |
| 93251_CD8 Lymphocytes_anti- | 0.0 |
| CD28/anti-CD3 | |
| 93353_chronic CD8 | 1.6 |
| Lymphocytes 2ry_resting dy 4- | |
| 6 in IL-2 | |
| 93574_chronic CD8 | 1.4 |
| Lymphocytes 2ry_activated | |
| CD3/CD28 | |
| 93354_CD4_none | 0.0 |
| 93252_Secondary | 1.7 |
| Th1/Th2/Tr1_anti-CD95 CH11 | |
| 93103_LAK cells_resting | 3.2 |
| 93788_LAK cells_IL-2 | 0.0 |
| 93787_LAK cells_IL-2 + IL-12 | 0.0 |
| 93789_LAK cells_IL-2 + IFN | 5.6 |
| gamma | |
| 93790_LAK cells_IL-2 + IL-18 | 0.7 |
| 93104_LAK | 0.0 |
| cells_PMA/ionomycin and IL- | |
| 18 | |
| 93578_NK Cells IL-2_resting | 0.0 |
| 93109_Mixed Lymphocyte | 2.9 |
| Reaction_Two Way MLR | |
| 93110_Mixed Lymphocyte | 2.7 |
| Reaction_Two Way MLR | |
| 93111_Mixed Lymphocyte | 0.0 |
| Reaction_Two Way MLR | |
| 93112_Mononuclear Cells | 1.3 |
| (PBMCs)_resting | |
| 93113_Mononuclear Cells | 0.0 |
| (PBMCs)_PWM | |
| 93114_Mononuclear Cells | 0.0 |
| (PBMCs)_PHA-L | |
| 93249_Ramos (B cell)_none | 2.1 |
| 93250_Ramos (B | 0.0 |
| cell)_ionomycin | |
| 93349_B lymphocytes_PWM | 0.0 |
| 93350_B lymphoytes_CD40L | 0.8 |
| and IL-4 | |
| 92665_EOL-1 | 18.4 |
| (Eosinophil)_dbcAMP | |
| differentiated | |
| 93248_EOL-1 | 0.0 |
| (Eosinophil)_dbcAMP/PMAion | |
| omycin | |
| 93356_Dendritic Cells_none | 2.9 |
| 93355_Dendritic Cells_LPS | 1.4 |
| 100 ng/ml | |
| 93775_Dendritic Cells_anti- | 4.5 |
| CD40 | |
| 93774_Monocytes_resting | 28.6 |
| 93776_Monocytes_LPS 50 | 3.3 |
| ng/ml | |
| 93581_Macrophages_resting | 1.3 |
| 93582_Macrophages_LPS 100 | 7.1 |
| ng/ml | |
| 93098_HUVEC | 0.0 |
| (Endothelial)_none | |
| 93099_HUVEC | 0.0 |
| (Endothelial)_starved | |
| 93100_HUVEC | 0.0 |
| (Endothelial)_IL-1b | |
| 93779_HUVEC | 0.0 |
| (Endothelial)_IFN gamma | |
| 93102_HUVEC | 0.0 |
| (Endothelial)_TNF alpha + IFN | |
| gamma | |
| 93101_HUVEC | 0.0 |
| (Endothelial)_TNF alpha + IL4 | |
| 93781_HUVEC | 0.0 |
| (Endothelial)_IL-11 | |
| 93583_Lung Microvascular | 0.7 |
| Endothelial Cells_none | |
| 93584_Lung Microvascular | 0.0 |
| Endothelial Cells_TNFa (4 | |
| ng/ml) and IL1b (1 ng/ml) | |
| 92662_Microvascular Dermal | 0.0 |
| endothelium_none | |
| 92663_Microsvasular Dermal | 1.5 |
| endothelium_TNFa (4 ng/ml) | |
| and IL1b (1 ng/ml) | |
| 93773_Bronchial | 0.0 |
| epithelium_TNFa (4 ng/ml) and | |
| IL1b (1 ng/ml)** | |
| 93347_Small Airway | 3.1 |
| Epithelium_none | |
| 93348_Small Airway | 6.5 |
| Epithelium_TNFa (4 ng/ml) | |
| and IL1b (1 ng/ml) | |
| 92668_Coronery Artery | 0.0 |
| SMC_resting | |
| 92669_Coronery Artery | 1.1 |
| SMC_TNFa (4 ng/ml) and IL1b | |
| (1 ng/ml) | |
| 93107_astrocytes_resting | 8.1 |
| 93108_astrocytes_TNFa (4 | 4.8 |
| ng/ml) and IL1b (1 ng/ml) | |
| 92666_KU-812 | 1.5 |
| (Basophil)_resting | |
| 92667_KU-812 | 1.7 |
| (Basophil)_PMA/ionoycin | |
| 93579_CCD1106 | 18.7 |
| (Keratinocytes)_none | |
| 93580_CCD1106 | 30.4 |
| (Keratinocytes)_TNFa and | |
| IFNg** | |
| 93791_Liver Cirrhosis | 41.9 |
| 93792_Lupus Kidney | 1.0 |
| 93577_NCI-H292 | 16.0 |
| 93358_NCI-H292_IL-4 | 8.7 |
| 93360_NCI-H292_IL-9 | 26.7 |
| 93359_NCI-H292_IL-13 | 9.3 |
| 93357_NCI-H292_IFN gamma | 2.9 |
| 93777_HPAEC_- | 1.4 |
| 93778_HPAEC_IL-1 beta/TNA | 0.0 |
| alpha | |
| 93254_Normal Human Lung | 0.0 |
| Fibroblast_none | |
| 93253_Normal Human Lung | 43.0 |
| Fibroblast_TNFa (4 ng/ml) and | |
| IL-1b (1 ng/ml) | |
| 93257_Normal Human Lung | 0.0 |
| Fibroblast_IL-4 | |
| 93256_Normal Human Lung | 3.3 |
| Fibroblast_IL-9 | |
| 93255_Normal Human Lung | 0.0 |
| Fibroblast_IL-13 | |
| 93258_Normal Human Lung | 0.0 |
| Fibroblast_IFN gamma | |
| 93106_Dermal Fibroblasts | 1.7 |
| CCD1070_resting | |
| 93361_Dermal Fibroblasts | 1.7 |
| CCD1070_TNF alpha 4 ng/ml | |
| 93105_Dermal Fibroblasts | 2.4 |
| CCD1070_IL-1 beta 1 ng/ml | |
| 93772_dermal fibroblast_IFN | 3.4 |
| gamma | |
| 93771_dermal fibroblast IL-4 | 1.4 |
| 93260_IBD Colitis 2 | 1.1 |
| 93261_IBD Crohns | 2.6 |
| 735010_Colon_normal | 100.0 |
| 735019_Lung_none | 24.0 |
| 64028-1_Thymus_none | 5.4 |
| 64030-1_Kidney_none | 6.0 |

Panel 1.3D Summary Ag1584 Expression of the NOV6 gene is limited to samples from bone marrow, spleen, and an ovarian cancer cell line (CTs=34). Thus, expression of this gene could be used to identify bone marrow and spleen tissue. In addition, expression of the SC NOV6 gene may be useful in identifying ovarian cancer cell lines.

Panel 2.2 Summary Ag1584 Expression of the NOV6 gene is limited to colon, breast and ovarian cancers. Thus, this gene could be used to distinguish colon, breast and ovarian cancers from normal tissue.

Panel 4D Summary Ag1584 Highest expression of the NOV6 gene in this sample is observed in the colon (CT=31.1). Low but significant levels of expression are also detected in in lung fibroblasts treated with the inflammatory cytokines TNF-a and IL-1b (CT=32.4) and keratinocytes treated with TNF-a and IFN g. The NOV6 gene has homology to the family of cold inducible glycoproteins (CIG 30) that are implicated in the synthesis of long chain fatty acids and sphingolipids. The sphingolipid ceramide is an important second signal molecule that regulates diverse signaling pathways involving apoptosis, cell senescence, the cell cycle, and differentiation. The production of ceramide is also important in programmed cell death. Ceramide levels are elevated in response to diverse stress challenges including chemotherapeutic drug treatment, irradiation, and treatment with pro-death ligands such as tumor necrosis factor alpha, TNF alpha. Therefore, therapeutic modulation of the expression of the NOV6 gene or the activity of its protein product, through the use of small molecule drugs or antibodies, may be important in the treatment of autoimmune diseases associated with increased apoptosis and other diseases associated with increased TNF-a production such as inflammatory bowel disease, rheumatoid arthritis and infectious diseases.

NOV7

Expression of gene NOV7 was assessed using the primer-probe sets Ag816 and Ag782 (identical sequences) described in Table 40. Results from RTQ-PCR runs are shown in Tables 41 and 42.

TABLE 40

Probe Name Ag816/Ag782 (identical sequences)

| | | | | |
|---|---|---|---|---|
| | | | | Start |
| Primers | Sequences | TM | Length | Position |

| | | | | |
|---|---|---|---|---|
| Forward | 5′-AGGAGGAGCTGGAGGAGAT-3′ (SEQ ID NO: 72) | 59.4 | 20 | 147 |
| Probe | TET-5′-AAGTCCGCCCACGTCTTCCACGT-3′-TAMRA | 72 | 23 | 180 |
| | (SEQ ID NO: 73) | | | |
| Reverse | 5′-ATCTTGTCGATGGCATTGAA-3′ (SEQ ID NO: 74) | 59.1 | 20 | 210 |

TABLE 41
Panel 1.2

| | Relative | | |
|---|---|---|---|
| | Expression (%) | Relative Expression (%) | |
| | 1.2tm957t_{—} | 1.2tm926t_{—} | 1.2tm1119t_{—} |
| Tissue Name | ag816 | ag782 | ag782 |
| Endothelial cells | 0.0 | 0.0 | 1.8 |
| Heart (fetal) | 0.0 | 0.2 | 75.3 |
| Pancreas | 0.4 | 0.4 | 0.1 |
| Pancreatic ca. CAPAN 2 | 0.0 | 0.0 | 1.1 |
| Adrenal Gland (new lot*) | 3.4 | 4.2 | 10.4 |
| Thyroid | 0.4 | 0.1 | 0.4 |
| Salivary gland | 0.8 | 0.6 | 3.4 |
| Pituitary gland | 100.0 | 100.0 | 18.2 |
| Brain (fetal) | 2.4 | 2.2 | 0.7 |
| Brain (whole) | 0.7 | 1.2 | 1.1 |
| Brain (amygdala) | 0.5 | 0.2 | 0.9 |
| Brain (cerebellum) | 2.8 | 2.6 | 2.4 |
| Brain (hippocampus) | 0.4 | 0.1 | 1.6 |
| Brain (thalamus) | 0.0 | 0.0 | 0.2 |
| Cerebral Cortex | 0.0 | 0.0 | 1.7 |
| Spinal cord | 1.0 | 1.0 | 1.8 |
| CNS ca. (glio/astro) | 0.4 | 0.3 | 4.6 |
| U87-MG | | | |
| CNS ca. (glio/astro) | 0.0 | 0.0 | 1.8 |
| U-118-MG | | | |
| CNS ca. (astro) SW1783 | 0.0 | 0.0 | 2.5 |
| CNS ca.* (neuro; met) | 1.0 | 0.8 | 1.8 |
| 5K-N-AS | | | |
| CNS ca. (astro) SF-539 | 0.0 | 0.0 | 0.1 |
| CNS ca. (astro) SNB-75 | 0.8 | 0.9 | 0.6 |
| CNS ca. (glio) SNB-19 | 0.0 | 0.0 | 3.1 |
| CNS ca. (glio) U251 | 0.0 | 0.0 | 0.2 |
| CNS ca. (glio) SF-295 | 0.0 | 0.0 | 1.7 |
| Heart | 0.0 | 0.0 | 100.0 |
| Skeletal Muscle (new lot*) | 2.7 | 4.0 | 6.1 |
| Bone marrow | 0.0 | 0.0 | 0.4 |
| Thymus | 1.0 | 1.0 | 1.0 |
| Spleen | 0.0 | 0.0 | 0.1 |
| Lymph node | 0.2 | 0.1 | 0.6 |
| Colorectal | 0.0 | 0.0 | 4.5 |
| Stomach | 1.9 | 2.2 | 4.3 |
| Small intestine | 1.7 | 1.7 | 2.4 |
| Colon ca. SW480 | 0.0 | 0.0 | 0.3 |
| Colon ca.* (SW480 met) | 0.0 | 0.0 | 0.1 |
| SW620 | | | |
| Colon ca. HT29 | 0.0 | 0.0 | 1.1 |
| Colon ca. HCT-116 | 0.0 | 0.0 | 1.7 |
| Colon ca. CaCo-2 | 0.0 | 0.0 | 1.0 |
| 83219 CC Well to | 0.1 | 0.0 | 2.9 |
| Mod Diff (ODO3866) | | | |
| Colon Ca. HCC-2998 | 0.0 | 0.0 | 0.8 |
| Gastric ca.* (liver met) | 0.6 | 0.7 | 8.4 |
| NCI-N87 | | | |
| Bladder | 0.9 | 2.2 | 4.2 |
| Trachea | 0.0 | 0.2 | 3.8 |
| Kidney | 2.8 | 2.6 | 9.9 |
| Kidney (fetal) | 1.3 | 0.8 | 7.1 |
| Renal ca. 786-0 | 0.0 | 0.0 | 2.7 |
| Renal ca. A498 | 2.4 | 3.3 | 9.5 |
| Renal ca. RXF 393 | 0.0 | 0.0 | 2.4 |
| Renal ca. ACHN | 0.0 | 0.0 | 9.6 |
| Renal ca. UO-31 | 0.0 | 0.0 | 7.6 |
| Renal ca. TK-10 | 14.8 | 18.3 | 18.8 |
| Liver | 0.0 | 0.0 | 1.0 |
| Liver (fetal) | 0.0 | 0.0 | 0.4 |
| Liver Ca. (hepatoblast) | 0.0 | 0.0 | 2.4 |
| HepG2 | | | |
| Lung | 0.0 | 0.0 | 0.0 |
| Lung (fetal) | 0.8 | 0.5 | 0.6 |
| Lung ca. (small cell) | 0.0 | 0.0 | 2.0 |
| LX-1 | | | |
| Lung ca. (small cell) | 1.4 | 2.8 | 5.8 |
| NCI-H69 | | | |
| Lung ca. (s.cell var.) | 21.0 | 23.8 | 21.9 |
| SHP-77 | | | |
| Lung ca. (large cell) | 5.1 | 6.7 | 9.5 |
| NCI-H460 | | | |
| Lung ca. (non-sm. cell) | 0.0 | 0.0 | 3.3 |
| A549 | | | |
| Lung ca. (non-s. cell) | 2.0 | 2.0 | 6.5 |
| NCI-H23 | | | |
| Lung ca (non-s. cell) | 0.0 | 0.0 | 1.4 |
| HOP-62 | | | |
| Lung ca. (non-s. cl) | 0.0 | 0.0 | 0.2 |
| NCI-H522 | | | |
| Lung ca. (squam.) | 1.2 | 1.9 | 13.8 |
| SW 900 | | | |
| Lung ca. (squam.) | 3.9 | 5.6 | 9.0 |
| NCI-H596 | | | |
| Mammary gland | 0.2 | 0.2 | 1.9 |
| Breast ca.* (pl. effusion) | 0.0 | 0.0 | 1.7 |
| MCF-7 | | | |
| Breast ca.* (pl.ef) | 0.0 | 0.0 | 1.7 |
| MDA-MB-231 | | | |
| Breast ca.* (pl. effusion) | 0.0 | 0.0 | 2.1 |
| T47D | | | |
| Breast ca. BT-549 | 0.0 | 0.0 | 0.9 |
| Breast ca. MDA-N | 0.4 | 0.3 | 3.2 |
| Ovary | 0.2 | 0.4 | 21.3 |
| Ovarian ca. OVCAR-3 | 0.0 | 0.0 | 0.5 |
| Ovarian ca. OVCAR-4 | 0.0 | 0.0 | 0.6 |
| Ovarian ca. OVCAR-5 | 4.2 | 5.7 | 19.6 |
| Ovarian ca. OVCAR-8 | 0.0 | 0.0 | 19.1 |
| Ovarian ca. IGROV-1 | 0.0 | 0.0 | 1.3 |
| Ovarian ca.* (ascites) | 0.0 | 0.0 | 4.9 |
| SK-OV-3 | | | |
| Uterus | 1.5 | 1.4 | 3.6 |
| Placenta | 0.0 | 0.0 | 16.2 |
| Prostate | 2.9 | 4.0 | 26.6 |
| Prostate ca.* (bone met) | 0.0 | 0.0 | 17.0 |
| PC-3 | | | |
| Testis | 1.8 | 0.9 | 3.3 |
| Melanoma Hs688 | 0.4 | 0.2 | 4.2 |
| (A).T | | | |
| Melanoma* (met) | 0.2 | 0.0 | 9.3 |
| Hs688(B).T | | | |
| Melanoma UACC-62 | 12.5 | 23.7 | 19.6 |
| Melanoma M14 | 0.0 | 0.0 | 2.4 |
| Melanoma LOX IMVI | 0.0 | 0.0 | 1.0 |
| Melanoma* (met) | 0.0 | 0.0 | 0.9 |
| SK-MEL-5 | | | |

TABLE 42
Panel 2D

| | Relative Expression(%) | |
|---|---|---|
| | 2Dtm2693t_{—} | 2Dtm2832t_{—} |
| Tissue Name | ag782 | ag782 |
| Normal Colon GENPAK 061003 | 8.3 | 9.0 |
| 83219 CC Well to Mod Diff (ODO3866) | 4.8 | 8.5 |
| 83220 CC NAT (ODO3866) | 3.8 | 5.6 |
| 83221 CC Gr.2 rectosigmoid (ODO3868) | 0.0 | 1.8 |
| 83222 CC NAT (ODO3868) | 3.4 | 1.8 |
| 83235 CC Mod Diff (ODO3920) | 1.0 | 2.4 |
| 83236 CC NAT (ODO3920) | 3.4 | 3.4 |
| 83237 CC Gr.2 ascend colon (ODO3921) | 4.8 | 14.6 |
| 83238 CC NAT (ODO3921) | 5.3 | 1.7 |
| 83241 CC from Partial Hepatectomy | 6.7 | 8.7 |
| (ODO4309) | | |
| 83242 Liver NAT (ODO4309) | 0.0 | 0.0 |
| 87472 Colon mets to lung (ODO4451-01) | 0.8 | 2.0 |
| 87473 Lung NAT (ODO4451-02) | 0.8 | 0.0 |
| Normal Prostate Clontech A+ 6546-1 | 25.2 | 23.5 |
| 84140 Prostate Cancer (ODO4410) | 28.1 | 31.6 |
| 84141 Prostate NAT (ODO4410) | 56.3 | 69.3 |
| 87073 Prostate Cancer (ODO4720-01) | 16.0 | 7.7 |
| 87074 Prostate NAT (ODO4720-02) | 35.1 | 39.0 |
| Normal Lung GENPAK 061010 | 2.8 | 0.0 |
| 83239 Lung Met to Muscle (ODO4286) | 1.6 | 2.0 |
| 83240 Muscle NAT (ODO4286) | 0.0 | 0.0 |
| 84136 Lung Malignant Cancer (ODO3126) | 100.0 | 100.0 |
| 84137 Lung NAT (ODO3126) | 0.9 | 0.5 |
| 84871 Lung Cancer (ODO4404) | 21.8 | 34.4 |
| 84872 Lung NAT (ODO4404) | 0.9 | 1.0 |
| 84875 Lung Cancer (ODO4565) | 7.5 | 5.7 |
| 84876 Lung NAT (ODO4565) | 0.0 | 1.9 |
| 85950 Lung Cancer (ODO4237-01) | 14.6 | 15.6 |
| 85970 Lung NAT (ODO4237-02) | 0.8 | 1.0 |
| 83255 Ocular Mel Met to Liver (ODO4310) | 0.9 | 0.0 |
| 83256 Liver NAT (ODO4310) | 0.0 | 0.0 |
| 84139 Melanoma Mets to Lung (ODO4321) | 68.8 | 71.2 |
| 84138 Lung NAT (ODO4321) | 0.0 | 0.0 |
| Normal Kidney GENPAK 061008 | 0.8 | 4.8 |
| 83786 Kidney Ca. Nuclear grade 2 | 14.3 | 14.1 |
| (ODO4338) | | |
| 83787 Kidney NAT (ODO4338) | 27.9 | 33.2 |
| 83788 Kidney Ca Nuclear grade | 0.9 | 0.8 |
| ½ (ODO4339) | | |
| 83789 Kidney NAT (ODO4339) | 7.0 | 9.5 |
| 83790 Kidney Ca. Clear cell type | 0.9 | 0.0 |
| (ODO4340) | | |
| 83791 Kidney NAT (ODO4340) | 6.9 | 4.0 |
| 83792 Kidney Ca, Nuclear grade 3 | 1.4 | 2.3 |
| (ODO4348) | | |
| 83793 Kidney NAT (ODO4348) | 7.5 | 5.0 |
| 87474 Kidney Cancer (ODO4622-01) | 55.1 | 37.9 |
| 87475 Kidney NAT (ODO4622-03) | 0.0 | 2.0 |
| 85973 Kidney Cancer (ODO4450-01) | 1.3 | 4.0 |
| 85974 Kidney NAT (ODO4450-03) | 4.7 | 4.2 |
| Kidney Cancer Clontech 8120607 | 7.8 | 9.0 |
| Kidney NAT Clontech 8120608 | 3.2 | 0.9 |
| Kidney Cancer Clontech 8120613 | 1.3 | 1.5 |
| Kidney NAT Clontech 8120614 | 7.5 | 15.3 |
| Kidney Cancer Clontech 9010320 | 6.7 | 25.5 |
| Kidney NAT Clontech 9010321 | 2.1 | 14.7 |
| Normal Uterus GENPAK 061018 | 2.2 | 1.9 |
| Uterus Cancer GENPAK 064011 | 5.2 | 3.5 |
| Normal Thyroid Clontech A+ 6570-1 | 3.7 | 2.2 |
| Thyroid Cancer GENPAK 064010 | 1.5 | 0.0 |
| Thyroid Cancer INVITROGEN A302152 | 7.2 | 9.2 |
| Thyroid NAT INVITROGEN A302153 | 1.6 | 2.4 |
| Normal Breast GENPAK 061019 | 2.8 | 1.0 |
| 84877 Breast Cancer (ODO4566) | 0.8 | 0.0 |
| 85975 Breast Cancer (ODO4590-01) | 3.7 | 2.2 |
| 85976 Breast Cancer Mets (ODO4590-03) | 3.4 | 0.9 |
| 87070 Breast Cancer Metastasis | 2.0 | 8.5 |
| (ODO4655-05) | | |
| GENPAK Breast Cancer 064006 | 5.8 | 5.1 |
| Breast Cancer Res. Gen. 1024 | 6.2 | 3.9 |
| Breast Cancer Clontech 9100266 | 13.8 | 8.3 |
| Breast NAT Clontech 9100265 | 3.1 | 2.2 |
| Breast Cancer INVITROGEN A209073 | 8.1 | 8.9 |
| Breast NAT INVITROGEN A2090734 | 1.7 | 1.8 |
| Normal Liver GENPAK 061009 | 0.0 | 0.0 |
| Liver Cancer GENPAK 064003 | 3.2 | 3.1 |
| Liver Cancer Research Genetics RNA 1025 | 0.9 | 1.1 |
| Liver Cancer Research Genetics RNA 1026 | 50.0 | 55.9 |
| Paired Liver Cancer Tissue Research | 1.6 | 0.0 |
| Genetics RNA 6004-T | | |
| Paired Liver Tissue Research Genetics | 2.5 | 2.5 |
| RNA 6004-N | | |
| Paired Liver Cancer Tissue Research | 61.1 | 49.7 |
| Genetics RNA 6005-T | | |
| Paired Liver Tissue Research Genetics | 1.0 | 0.0 |
| RNA 6005-N | | |
| Normal Bladder GENPAK 061001 | 17.2 | 5.6 |
| Bladder Cancer Research Genetics RNA | 3.8 | 1.9 |
| 1023 | | |
| Bladder Cancer INVITROGEN A302173 | 30.4 | 35.6 |
| 87071 Bladder Cancer (ODO4718-01) | 3.3 | 4.8 |
| 87072 Bladder Normal Adjacent | 2.4 | 0.9 |
| (ODO4718-03) | | |
| Normal Ovary Res. Gen. | 4.1 | 2.2 |
| Ovarian Cancer GENPAK 064008 | 67.4 | 84.1 |
| 87492 Ovary Cancer (ODO4768-07) | 4.0 | 5.4 |
| 87493 Ovary NAT (ODO4768-08) | 2.9 | 3.8 |
| Normal Stomach GENPAK 061017 | 2.4 | 5.4 |
| Gastric Cancer Clontech 9060358 | 0.0 | 0.7 |
| NAT Stomach Clontech 9060359 | 0.0 | 0.0 |
| Gastric Cancer Clontech 9060395 | 2.8 | 2.1 |
| NAT Stomach Clontech 9060394 | 3.4 | 0.5 |
| Gastric Cancer Clontech 9060397 | 1.9 | 4.9 |
| NAT Stomach Clontech 9060396 | 1.0 | 0.0 |
| Gastric Cancer GENPAK 064005 | 2.9 | 6.4 |

Summary Panel 1.2 Ag816/Ag782 Multiple experiments with the same probe and primer set show widespread expression of the NOV7 gene throughout many of the tissues in this panel. Highest expression is observed in the pituitary gland (CT=22.5). Thus, this gene could be used to distinguish pituitary tissue from other glandular tissues.

Among metabolically relevant tissues, the NOV7 gene is expressed in the pituitary, pancreas, adrenal gland, thyroid, skeletal muscle and adult heart. Since the NOV7 gene is not expressed in fetal heart tissue, expression of the gene could be used as a marker to distinguish between the two tissue types.

For tissues active in the central nervous system, the NOV7 gene is expressed in the brain at moderate levels and is localized to the amygdala, cerebellum, hippocampus, thalamus, cerebral cortex, spinal cord and the developing brain. The NOV7 gene, a homolog of matrilin-2, appears to be an intercellular matrix protein. Glial scarring is a major inhibitor of CNS repair and regeneration and involves intracellular and extracellular proteins. Thus, reduction of levels of this protein encoded by the NOV7 gene may decrease levels of glial scarring in response to CNS injury, and promote healing in spinal cord and/or brain trauma.

The NOV7 gene is also expressed in the prostate (CT=27.6) and in kidney cancer cell lines and lung cancer cell lines. Thus, the expression of this gene could be used to distinguish prostate tissue from other tissue, and kidney and lung cancer cell lines from other cell lines. Moreover, therapeutic modulation of the expression of this gene or the function of its product, through the use of small molecule drugs, antibodies or protein therapeutics, might be usefule in the treatment of kidney or lung cancer. The NOV7 gene is also detected in fetal lung (CT=29.5), but not in adult lung tissue. Thus, expression of the NOV7 gene could be used to differentiate between the two tissue types.

Summary Panel 2D Ag782 Results from two experiments using the same probe/primer set are in good agreement. The NOV7 gene appears to be overexpressed in ovarian, lung, breast, and liver cancers as compared to their normal adjacent tissues. Thus, the expression of this gene could be used to distinguish lung, ovarian, breast and liver cancers from other cancers. Moreover, the expression of the NOV7 gene could be used to distinguish the cancerous forms of lung, liver, breast and ovary. Finally, therapeutic modulation of this gene or gene product, through the use of small molecule drugs, antibodies or protein therapeutics, might be of benefit for the treatment of lung, liver, ovarian or breast cancer.

References

1. Deak F, Piecha D, Bachrati C, Paulsson M, Kiss I. (1997) Primary structure and expression of matrilin-2, the closest relative of cartilage matrix protein within the von Willebrand factor type A-like module superfamily. J Biol Chem. 272:9268-74.

A mouse cDNA encoding a novel member of the von Willebrand factor type A-like module superfamily was cloned. The protein precursor of 956 amino acids consists of a putative signal peptide, two von Willebrand factor type A-like domains connected by 10 epidermal growth factor-like modules, a potential oligomerization domain, and a unique segment, and it contains potential N-glycosylation sites. A sequence similarity search indicated the closest relation to the trimeric cartilage matrix protein (CMP). Since they constitute a novel protein family, we introduce the term matrilin-2 for the new protein, reserving matrilin-1 as an alternative name for CMP. A 3. 9-kilobase matrilin-2 mRNA was detected in a variety of mouse organs, including calvaria, uterus, heart, and brain, as well as fibroblast and osteoblast cell lines. Expressed human and rat cDNA sequence tags indicate a high degree of interspecies conservation. A group of 120-1 50-kDa bands was, after reduction, recognized specifically with an antiserum against the matrilin-2-glutathione S-transferase fusion protein in media of the matrilin-2-expressing cell lines. Assuming glycosylation, this agrees well with the predicted minimum Mr of the mature protein (104,300). Immunolocalization of matrilin-2 in developing skeletal elements showed reactivity in the perichondrium and the osteoblast layer of trabecular bone. CMP binds both collagen fibrils and aggrecan, and because of the similar structure and complementary expression pattern, matrilin-2 is likely to perform similar functions in the extracellular matrix assembly of other tissues.

NOV8

Expression of gene SC65666665_A was assessed using the primer-probe sets Ag1633 and Ag1535 described in Tables 43 and 44. Results from RTQ-PCR runs are shown in Table 45.

TABLE 43

Probe Name Ag1633

| | | | | |
|---|---|---|---|---|
| | | | | Start |
| Primers | Sequences | TM | Length | Position |

| | | | | | |
|---|---|---|---|---|---|
| Forward | 5′-GTGAAAGGGTGCTATGCAAA-3′ | (SEQ ID NO: 75) | 58.8 | 20 | 494 |
| Probe | FAM-5′-CTGTGGTTTCACGCCAATTTCCTGTA-3′-TAMRA | (SEQ ID NO: 76) | 68.8 | 26 | 521 |
| Reverse | 5′-CCACCTGGATTGCACATATTA-3′ | (SEQ ID NO: 77) | 58.4 | 21 | 570 |

TABLE 44

Probe Name Ag1535

| | | | | |
|---|---|---|---|---|
| | | | | Start |
| Primers | Sequences | TM | Length | Position |

| | | | | |
|---|---|---|---|---|
| Forward | 5′-GTGAAAGGGTGCTATGCAAA-3′ | 58.8 | 20 | 494 |
| Probe | TET-5′-CTGTGGTTTCACGCCAATTTCCTGTA-3′-TAMRA | 68.8 | 26 | 521 |
| Reverse | 5′-ACCACCTGGATTGCACATATTA-3′ | 59.2 | 22 | 570 |

TABLE 45
Panel 1.2

| | Relative |
|---|---|
| | Expression (%) |
| | 1.2tm2181t_{—} |
| Tissue Name | ag1535 |
| Endothelial cells | 0.0 |
| Heart (fetal) | 2.8 |
| Pancreas | 2.8 |
| Pancreatic ca. CAPAN 2 | 0.0 |
| Adrenal Gland (new lot*) | 0.0 |
| Thyroid | 0.0 |
| Salivary gland | 6.1 |
| Pituitary gland | 0.0 |
| Brain (fetal) | 6.7 |
| Brain (whole) | 9.7 |
| Brain (amygdala) | 9.4 |
| Brain (cerebellum) 7.6 | |
| Brain (hippocampus) | 48.6 |
| Brain (thalamus) | 8.4 |
| Cerebral Cortex | 25.9 |
| Spinal cord | 0.0 |
| CNS ca. (glio/astro) U87-MG | 2.4 |
| CNS ca. (glio/astro) U-118-MG | 2.0 |
| CNS ca. (astro) SW1783 | 0.0 |
| CNS ca.* (neuro; met) SK-N- | 0.0 |
| AS | |
| CNS ca. (astro) SF-539 | 0.0 |
| CNS ca. (astro) SNB-75 | 0.0 |
| CNS ca. (glio) SNB-19 | 0.0 |
| CNS ca. (glio) U251 | 0.0 |
| CNS ca. (glio) SF-295 | 4.2 |
| Heart | 100.0 |
| Skeletal Muscle (new lot*) | 11.7 |
| Bone marrow | 2.8 |
| Thymus | 0.0 |
| Spleen | 6.9 |
| Lymph node | 0.0 |
| Colorectal | 0.0 |
| Stomach | 2.0 |
| Small intestine | 6.5 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| 83219 CC Well to Mod Diff | 0.0 |
| (ODO3866) | |
| Colon ca. HCC-2998 | 13.0 |
| Gastric ca.* (liver met) NCI- | 14.1 |
| N87 | |
| Bladder | 0.0 |
| Trachea | 0.0 |
| Kidney | 54.7 |
| Kidney (fetal) | 0.0 |
| Renal ca. 786-0 | 3.2 |
| Renal ca. A498 | 2.6 |
| Renal ca. RXF 393 | 4.5 |
| Renal ca. ACHN | 2.9 |
| Renal ca. UO-31 | 0.0 |
| Renal ca. TK-10 | 4.6 |
| Liver | 8.7 |
| Liver (fetal) | 0.0 |
| Liver ca. (hepatoblast) HepG2 | 6.6 |
| Lung | 0.0 |
| Lung (fetal) | 0.0 |
| Lung ca. (small cell) LX-1 | 0.0 |
| Lung ca. (small cell) NCI-H69 | 6.6 |
| Lung ca. (s. cell var.) SHP-77 | 0.0 |
| Lung ca. (large cell) NCI-H460 | 7.6 |
| Lung ca. (non-sm. cell) A549 | 5.9 |
| Lung ca. (non-s. cell) NCI-H23 | 3.7 |
| Lung ca (non-s. cell) HOP-62 | 10.9 |
| Lung ca. (non-s. cl.) NCI-H522 | 15.6 |
| Lung ca. (squam.) SW 900 | 7.4 |
| Lung ca. (squam.) NCI-H596 | 0.0 |
| Mammary gland | 0.0 |
| Breast ca.* (pl. effusion) MCF- | 0.0 |
| 7 | |
| Breast ca.* (pl. ef) MDA-MB- | 0.0 |
| 231 | |
| Breast ca.* (pl. effusion) T47D | 2.6 |
| Breast ca. BT-549 | 2.8 |
| Breast ca. MDA-N | 0.0 |
| Ovary | 3.0 |
| Ovarian ca. OVCAR-3 | 2.6 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 4.1 |
| Ovarian ca.* (ascites) SK-OV-3 | 0.0 |
| Uterus | 3.1 |
| Placenta | 0.0 |
| Prostate | 8.0 |
| Prostate ca.* (bone met) PC-3 | 0.0 |
| Testis | 0.0 |
| Melanoma Hs688(A).T | 0.0 |
| Melanoma* (met) Hs688(B).T | 0.0 |
| Melanoma UACC-62 | 0.0 |
| Melanoma M14 | 6.5 |
| Melanoma LOX IMVI | 0.0 |
| Melanoma* (met) SK-MEL-5 | 0.0 |

Summary Panel 1.2 Ag1535 Significant expression of the NOV8 gene is limited to adult heart tissue (CT=34.1). Thus, expression of this gene could be used to distinguish adult heart tissue from fetal heart tissue. The protein encoded by the NOV8 gene may also be an antibody target for the treatment of cardiovascular diseases.

Expression of this gene in panels 1.3D, 2D, and 4D using the probe/primer set Ag1633 is low/undetectable (Ct values>35) in all samples on these panels (data not shown).

NOV9

Expression of gene NOV9 was assessed using the primer-probe sets Ag1554, described in Table 46.

TABLE 46

Probe Name Ag1554

| | | | | |
|---|---|---|---|---|
| | | | | Start |
| Primers | Sequences | TM | Length | Position |

| | | | | | |
|---|---|---|---|---|---|
| Forward | 5′-ACATCCTCACGGAACTCATG-3′ | (SEQ ID NO: 78) | 58.5 | 20 | 958 |
| Probe | FAM-5′-AGTGGCTCTGCTCCACTCCCCTCT-3′-TAMRA | (SEQ ID NO: 79) | 69.9 | 24 | 1008 |
| Reverse | 5′-GGCAGGACTTTCTCATCAGAGT-3′ | (SEQ ID NO: 80) | 59.9 | 22 | 1036 |

xpression of this gene in panels 4D and CNS_neurodegeneration is low/undetectable (Ct values>35) in all samples on these panels.

### Example 3

### SNP Analysis of NOVX Clones

SeqCallingTM Technology: cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, MA). Detailed protocols for Pyrosequencing can be found in: Alderborn et al. Determination of Single Nucleotide Polymorphisms by Real-time Pyrophosphate DNA Sequencing. (2000). Genome Research. 10, Issue 8, August. 1249-1265. In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on an indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels. The DNA and protein sequences for the novel single nucleotide polymorphic variants are reported. Variants are reported individually but any combination of all or a select subset of variants are also included. In addition, the positions of the variant bases and the variant amino acid residues are underlined.

Results

Variants are reported individually but any combination of all or a select subset of variants are also included as contemplated NOVX embodiments of the invention.

NOV1 SNP data

NOV1 has one SNP variant, whose variant positions for its nucleotide and amino acid sequences is numbered according to SEQ ID NOs: 1 and 2, respectively. The nucleotide sequence of the NOV1 variant differs as shown in Table 47.

TABLE 47
cSNP and Coding Variants for NOV1

| NT Position | Wild Type | | Amino Acid | Amino Acid |
|---|---|---|---|---|
| of cSNP | NT | Variant NT | position | Change |
| 1319 | A | C | 431 | Thr to Pro |

NOV2c SNP data

NOV2c has one SNP variant, whose variant positions for its nucleotide and amino acid sequences is numbered according to SEQ ID NOs: 7 and 8, respectively. The nucleotide sequence of the NOV2c variant differs as shown in Table 48.

TABLE 48
cSNP and Coding Variants for NOV2c

| NT Position | Wild Type | | Amino Acid | Amino Acid |
|---|---|---|---|---|
| of cSNP | NT | Variant NT | position | Change |
| 878 | A | G | | None |

NOV4 SNP data

NOV4 has three SNP variants, whose variant positions for its nucleotide and amino acid sequences is numbered according to SEQ ID NOs: 13 and 14, respectively. The nucleotide sequence of the NOV4 variant differs as shown in Table 49.

TABLE 49
cSNP and Coding Variants for NOV4

| NT Position | Wild Type | | Amino Acid | Amino Acid |
|---|---|---|---|---|
| of cSNP | NT | Variant NT | position | Change |
| 7 | A | G | | None |
| 169 | T | C | 40 | Val to Ala |
| 276 | T | C | 76 | Pro to Ser |

NOV8 SNP data

NOV8 has four SNP variants, whose variant positions for its nucleotide and amino acid sequences is numbered according to SEQ ID NOs: 23 and 24, respectively. The nucleotide sequence of the NOV8 variant differs as shown in Table 50.

TABLE 50
cSNP and Coding Variants for NOV8

| NT Position | Wild Type | | Amino Acid | Amino Acid |
|---|---|---|---|---|
| of cSNP | NT | Variant NT | position | Change |
| 321 | A | G | 107 | Lys to Arg |
| 527 | T | G | 176 | Phe to Val |
| 531 | A | G | 177 | His to Arg |
| 622 | C | T | | None |

NOV9 SNP data

NOV9 has one SNP variant, whose variant positions for its nucleotide and amino acid sequences is numbered according to SEQ ID NOs:25 and 26, respectively. The nucleotide sequence of the NOV9 variant differs as shown in Table 51.

TABLE 51
cSNP and Coding Variants for NOV9

| NT Position | Wild Type | | Amino Acid | Amino Acid |
|---|---|---|---|---|
| of cSNP | NT | Variant NT | position | Change |
| 1532 | C | T | | None |

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims.

What is claimed is:

1. An isolated polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24 and 26;
(b) a variant of a mature form of an amino acid sequence selected from the group consisting of SEQ ID NOS: 2, 4, 6, 8, 10, 12, 14, 16, 18, 20, 22, 24 and 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting SEQ ID NOS: 2, 4, 6, 8, 10, 12, 14, 16, 18, 20, 22, 24 and 26; and
(d) a variant of an amino acid sequence selected from the group consisting of SEQ ID NOS: 2, 4, 6, 8, 10, 12, 14, 16, 18, 20, 22, 24 and 26, wherein one or more amino acid residues in said variant differs from the amino acid sequence of said mature form, provided that said variant differs in no more than 15% of amino acid residues from said amino acid sequence.

2. The polypeptide of claim 1, wherein said polypeptide comprises the amino acid sequence of a naturally-occurring allelic variant of an amino acid sequence selected from the group consisting SEQ ID NOS: 2, 4, 6, 8, 10, 12, 14, 16, 18, 20, 22, 24 and 26.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS: 1, 3, 5, 7,9, 11, 13, 15, 17, 19, 21, 23, and 25.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS: 2, 4, 6, 8, 10, 12, 14, 16, 18, 20, 22, 24 and 26;
(b) a variant of a mature form of an amino acid sequence selected from the group consisting of SEQ ID NOS: 2, 4, 6, 8, 10, 12, 14, 16, 18, 20, 22, 24 and 26, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS: 2, 4, 6, 8, 10, 12, 14, 16, 18, 20, 22, 24 and 26;
(d) a variant of an amino acid sequence selected from the group consisting SEQ ID NOS: 2, 4, 6, 8, 10, 12, 14, 16, 18, 20, 22, 24 and 26, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS: 2, 4, 6, 8, 10, 12, 14, 16, 18, 20, 22, 24 and 26, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (e).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS: 1, 3, 5, 7, 9, 11, 13, 15, 17, 19, 21, 23, and 25.

9. The nucleic acid molecule of claim 5, wherein said nucleic acid molecule comprises a nucleotide sequence selected from the group consisting of:
(a) a nucleotide sequence selected from the group consisting of SEQ ID NOS: 1, 3, 5, 7, 9, 11, 13, 15, 17, 19, 21, 23, and 25;
(b) a nucleotide sequence differing by one or more nucleotides from a nucleotide sequence selected from the group consisting of SEQ ID NOS: 1, 3, 5, 7, 9, 11, 13, 15, 17, 19, 21, 23, and 25, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting SEQ ID NOS: 1, 3, 5, 7, 9, 11, 13, 15, 17, 19, 21, 23, and 25, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide,
whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a NOVX-associated disorder, said method comprising administering to a subject in which such treatment or prevention is desired the polypeptide of claim 1 in an amount sufficient to treat or prevent said NOVX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a NOVX-associated disorder, said method comprising administering to a subject in which such treatment or prevention is desired the nucleic acid of claim 5 in an amount sufficient to treat or prevent said NOVX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a NOVX-associated disorder, said method comprising administering to a subject in which such treatment or prevention is desired the antibody of claim 15 in an amount sufficient to treat or prevent said NOVX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease;
wherein an alteration in the expression level of the polypeptide in the first subject as compared to the control sample indicates the presence of or predisposition to said disease.

45. The method of claim 44 wherein the predisposition is to a cancer.

46. A method for determining the presence of or predisposition to a disease associated with altered levels of the nucleic acid molecule of claim 5 in a first mammalian subject, the method comprising:
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease;
wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS: 2, 4, 6, 8, 10, 12, 14, 16, 18, 20, 22, 24 and 26, or a biologically active fragment thereof.

49. A method of treating a pathological state in a mammal, the method comprising administering to the mammal the antibody of claim 15 in an amount sufficient to alleviate the pathological state.
